(19) United States
(12) Patent Application Publication
(10) Pub. No.: US 2025/0011379 A1
(43) Pub. Date: Jan. 9, 2025

(54) COMPLEMENT FACTOR I-RELATED COMPOSITIONS AND METHODS

(71) Applicant: Vertex Pharmaceuticals Incorporated, Boston, MA (US)

(72) Inventors: Grant E. BLOUSE, Burlingame, CA (US); Jan Kristian JENSEN, Randers (DK); Emil OLDENBURG, Aarhus (DK); Christine René SCHAR, Randers (DK); Agnieszka JENDROSZEK, Risskov (DK); James N. MCGUIRE, South San Francisco, CA (US); Shyam Rajan IYER, Woodside, CA (US); Kyle A. PELOT, Fremont, CA (US)

(21) Appl. No.: 18/750,921

(22) Filed: Jun. 21, 2024

Related U.S. Application Data
(63) Continuation of application No. PCT/US2022/082177, filed on Dec. 21, 2022.
(60) Provisional application No. 63/293,040, filed on Dec. 22, 2021.

(51) Int. Cl. C07K 14/47; A61K 47/60; A61P 37/06; C07K 14/76

(52) U.S. Cl. CPC C07K 14/4703; A61K 47/60; A61P 37/06; C07K 14/76; C07K 2319/30

(57) ABSTRACT

Provided herein are Complement Factor I (CFI) variants and CFI containing fusion constructs that exhibit at least one improved characteristic relative to a wild type CFI. The CFI variants and fusion constructs of the disclosure can exhibit tunable specificity and activity. The CFI variants and fusion constructs provided herein may be useful for treating a disease or condition associated with dysregulation of the complement system or a deficiency of CFI.

### CROSS REFERENCE TO RELATED APPLICATION

This application claims priority to U.S. Provisional Application No. 63/293,040 filed on Dec. 22, 2021, the contents of which are incorporated herein by reference in their entireties.

### REFERENCE TO AN ELECTRONIC SEQUENCE LISTING

The contents of the electronic sequence listing (VTEX_706_01WO_SeqList_ST26.xml; Size: 53,828 bytes; and Date of Creation: Dec. 20, 2022) are herein incorporated by reference in its entirety.

### BACKGROUND

The complement system includes the classical, lectin and alternative pathways, and is tightly controlled by a number of regulators. Complement Factor I (CFI) is one such regulator, and acts to regulate the complement system by cleaving C4b and C3b proteins, thereby inactivating these proteins. Such cleavage results in inhibition of the classical and lectin pathways (via cleavage of C4b), and inhibition of the alternative pathway (via cleavage of C3b), thus ultimately preventing the assembly of the C3 and C5 convertase enzymes.

CFI is encoded as a proenzyme that is then activated by proteolytic cleavage into a heterodimeric glycoprotein, the heterodimeric protein having a heavy chain and a light chain that are connected by a disulfide linkage. The light chain (also referred to as the B chain) comprises the serine protease domain (SPD) responsible for the cleavage of C3b and C4b, and contains a catalytic triad (His362, Asp411, and Ser507) within a region referred to as the active site. The heavy chain (also referred to as the A chain) comprises four domains: the FI membrane attack complex (FIMAC) domain, the scavenger receptor cysteine-rich domain SRCR (also called the CD5 domain) domain, the low density lipoprotein receptor 1 domain (LDLr1), and the low density lipoprotein receptor 2 domain (LDLr2). CFI is processed into its active form post-translationally by the addition of six Asn-linked glycans and proteolytic activation by furin, thereby excising a RRKR linker (SEQ ID NO: 25) to generate the two chain mature protein.

With respect to its ability to cleave C3b or C4b, CFI is proteolytically active when it forms ternary complexes with its cofactors; Factor H (FH) or Complement Receptor 1 (CR1, also called CD35) and its physiological substrates, C3b and C4b. FH is an example of a soluble member of the group of proteins called regulators of complement activation (RCA). The formation of the complex made by CFI and FH and subsequent cleavage of C3b together act to regulate the alternative pathway of the complement system. Continuous regulation of C3b levels by CFI acts to maintain the balance between the classical and alternative pathways. For instance, removal of CFI has been shown to cause an immediate activation, resulting in over-activity, of the alternative pathway. CRI is an example of a monomeric single-pass type I membrane glycoprotein and is also a member of the RCA group of proteins. Formation of the complex made between CFI and CRI and the subsequent cleavage of C3b and C4b act to regulate the alternative or the classical and lectin pathways, respectively.

Dysregulated CFI, mutated and dysfunctional CFI, and CFI deficiency have been implicated in diseases involving the complement system. Needed are methods for modulating or inhibiting particular points of regulation within the complement system. Provided here are compositions and methods to address this need.

### SUMMARY

In one aspect, the disclosure provides complement factor I (CFI) variants comprising at least one modification with respect to a wild type CFI, and fusion proteins comprising such CFI variants. Exemplary CFI variants and fusion proteins are provided in Tables 2 and 3. The CFI variants of the disclosure are capable of modulating the complement system, and exhibit at least one improved characteristic as compared to a wild type CFI. Also provided are methods of making and using the CFI variants and fusion proteins of the disclosure.

### BRIEF DESCRIPTION OF THE DRAWINGS

FIG. 1 is a schematic representation of the domains of a wild type Complement Factor I (CFI), showing a heavy chain and a light chain. The heavy chain (A Chain) includes the FIMAC, SRCR, LDLr1, LDLr2 domains, and a linker. The light chain (B Chain) includes the serine protease domain. Regions of the light chain include the activation loop, 37-Loop, 60-Loop, 70-Loop, 99-Loop, 110-Loop, 150-Autolysis Loop, 180-Loop Oxyanion Stabilizing, and/or 220-Loop S1 Entrance Frame.

FIGS. 2A-2D depict exemplary models of fusion constructs of the disclosure between albumin (e.g. serum albumin, e.g. human serum albumin (HSA)) and CFI comprising a CFI variant, wherein the CFI variant comprises an A-B chain inversion.

FIG. 3A depicts a model of an exemplary CFI-albumin (e.g. serum albumin, e.g. human serum albumin (HSA)) fusion construct comprising serum albumin fused with CFI.

FIG. 3B depicts a model of an exemplary CFI-HSA fusion construct comprising HSA fused with the serine protease domain of a CFI.

FIG. 4A depicts a schematic diagram of Factor H (FH) showing its 20 domains.

FIG. 4B depicts a schematic diagram of a mini Factor H showing domains 1-4 connected to domains 19-20 of FH.

FIG. 5 depicts a model of an exemplary fusion construct comprising portions of Factor H and CFI, comprising domains 1-8 of FH fused with CFI.

FIG. 6 depicts a schematic representation of three exemplary fusion constructs of the disclosure, each comprising HSA, at least one CFI domain, and various domains of Factor H, the part connected by optional/exemplary linkers.

FIG. 7 depicts a schematic representation of an exemplary CFI fusion construct, wherein the “Core” domain comprises any one or more of the CFIs of the disclosure, and may be flanked by one or more domains in either or both the N and C termini. The domains depicted may be optionally connected by linkers.

FIGS. 8A-8B are graphs depicting the measured concentrations of a wild type CFI-HSA fusion construct as compared to free plasma purified CFI, after a single subcutaneous administration to monkeys at a dose of 1 mg/kg.

FIGS. 9A-9B depict graphs of C3b degradation and C4b degradation, respectively, by CFI-HSA and plasma-derived CFI.

FIGS. 9C-9D depict graphs of hemolytic assays of CFI-HSA and plasma-derived CFI. Wherein AP represents an alternative pathway focused assay and CP+AP represents an alternative and classical pathway focused assay.

FIG. 10 depicts a plot showing the amount of C3a detected in samples from the vitreous humor following intravitreal injection (IVT) of CFI-HSA at a dose of 250 μg or 500 μg in an African green monkey primate model.

FIG. 11 depicts a plot of Factor I levels detected in plasma at the indicated time points following intravenous injection of CFI-HSA (Factor I-HSA) at 3 mg/kg or CFI (Factor I) at 1.3 mg/kg in CD1 mice.

FIG. 12 depicts a plot of Factor I levels detected in plasma at the indicated time points following subcutaneous injection of CFI-HSA (Factor I-HSA) at 3 mg/kg or CFI (Factor I) at 6.5 mg/kg in CD1 mice.

### DETAILED DESCRIPTION

The disclosure provides compositions and methods useful for modulating the signaling and amplification of the complement system. By providing complement factor I (CFI) variants and CFI-containing fusion constructs that are more or less active on one or more physiological substrates of CFI, and/or more stable than plasma-derived CFI, a modulation of the complement system is observed. Such modulation includes an increased amount of C3b cleavage and/or C4b cleavage, thus reducing complement activation, and reducing the amplification of the complement pathways. For example, some CFI variants can alter levels of regulators within the complement system. In some embodiments, the CFI variants and fusion constructs provided herein can act on the classical and lectin pathways of the complement system, on the alternative pathway of the complement system, or on both pathways. The disclosure also provides methods of making and using these variants and constructs, for example in treating a disease or condition associated with complement dysregulation, e.g. treating an overactive complement response.

### I. Complement Factor I Proteins Useful for Modulation of the Complement System

### A. Complement Factor I Variants

Provided herein are Complement Factor I variants (CFI), such variants comprising one or more modifications with respect to a wild type CFI, referred to herein as “CFI variants.” As used herein, a “modification” to a wild type CFI includes: a deletion of one or more amino acid residues, a deletion of one or more domains, a substitution of one or more amino acid residues, an insertion (i.e. addition) of one or more amino acid residues, an insertion (i.e. addition) of one or more domains, an inversion of or one or more domains, and a substitution of one or more domains.

The CFI variants of the disclosure do not directly act on C3, for example, the variants of the disclosure do not directly cleave C3, do not directly inhibit C3, do not directly inhibit the activation of C3, and do not directly reduce the activation of C3.

As used herein, a “wild type CFI” refers to any naturally occurring full-length CFI that is not a disease-causing CFI, with or without a signal sequence, and which may be of any species.

In some embodiments, a wild type CFI is plasma-derived. In some embodiments, a wild type CFI is a human wild type CFI. In some embodiments, a wild type, human CFI having a signal sequence comprises the amino acid sequence set forth in SEQ ID NO: 1 (as shown in Table 1 below). In some embodiments, a wild type CFI is a human CFI. In some embodiments, a wild type, human CFI does not include a signal sequence. In some embodiments, a wild type CFI without a signal sequence comprises the amino acid sequence set forth in SEQ ID NO: 5 (as shown in Table 1 below).

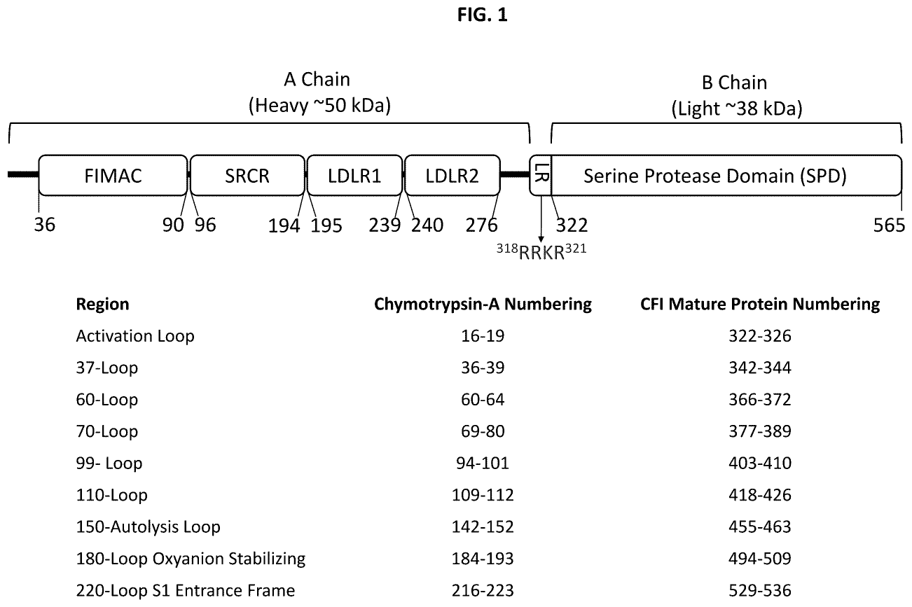

A wild type CFI comprises a heavy chain and a light chain, which are also referred to as the A-chain and B-chain, respectively. FIG. 1 depicts a schematic diagram of CFI showing the two chains. The heavy chain (A-chain) has four domains: the FI membrane attack complex (FIMAC) domain (residues 36 to 90 of SEQ ID NO: 5), the SRCR domain is further composed of a plurality of scavenger receptor cysteine-rich (SRCR) domains, a low density lipoprotein 1 domain (LDLr1), and a low density lipoprotein 2 domain (LDLr2). The light chain (B-chain) consists of a serine protease domain (SPD). The interface between these chains is referred to as the A:B chain interface.

A CFI variant of the disclosure includes one or more of a deletion of one or more amino acid residues of a wild type CFI, a deletion of one or more CFI domains of a wild type CFI, a substitution of one or more amino acid residues of a wild type CFI, an insertion of one or more amino acid residues to a wild type CFI, an inversion of one or more CFI domains of a wild type CFI, and an insertion of one or more domains to a wild type CFI.

The CFI variants of the disclosure may be generated by the introduction of one or more modifications to a CFI base molecule, wherein the domains of the CFI base molecule correspond to those domains found in a wild type CFI, e.g. as put forth in FIG. 1. A CFI base molecule may therefore be a wild type CFI of any species, or a CFI base molecule may comprise only portions of a wild type CFI, having only some of the domains of a wild type CFI of any species (e.g. already a CFI variant). In some embodiments, a CFI base molecule is a wild type, mouse CFI. In some embodiments, a CFI base molecule is a wild type, human CFI. In some embodiments, a CFI base molecule is a wild type, non-human primate CFI. In some embodiments, a CFI base molecule comprises only some domains of a wild type, human CFI.

In some embodiments, the CFI variants provided herein modulate the activity of the complement system and have at least one improved characteristic as compared to a wild type CFI. Such improved characteristics include, but are not limited to an increase or decrease in any one or more of bioavailability, half-life, activity, potency, catalytic capability, cofactor affinity (e.g. affinity for Factor H and/or CR1), substrate specificity and substrate affinity (e.g. affinity for C3b and/or C4b). In some embodiments, the improved characteristic is increased half-life. In some embodiments, the improved characteristic is an increase in activity, discussed further in detail, in subsequent sections below. In other embodiments, the improved characteristic is a change in substrate specificity for C3b and/or C4b, allowing for tunability of the CFI variant.

Provided in Table 1 are exemplary base molecules that may be used for the generation of CFI variants. The base molecules provided herein may be useful for modulation of the complement system without further modification, or may be useful for modulation of the complement system with further modification. For example, any one of the base molecules provided in Table 1 may be further modified to include one or more modifications, such as a deletion of one or more amino acid residues, a deletion of one or more CFI domains, a substitution of one or more amino acid residues, or an addition of one or more amino acid residues or CFI domains. The base molecules of Table 1 may be further part of a fusion construct, further described below.

TABLE 1
Base Molecules for Generation of CFI Variants

| Description | | |
|---|---|---|
| of | Nomenclature | |
| CFI Base | of Base | Amino Acid |
| Molecule | Molecule | Sequence |
| Plasma derived, | CFI-PD | MKLLHVFLLFLCFHL |
| wild type | | RFCKVTYTSQEDLVE |
| human CFI | | KKCLAKKYTHLSCDK |
| (wt hCFI) | | VFCQPWQRCIEGTCV |
| (with signal | | CKLPYQCPKNGTAVC |
| sequence | | ATNRRSFPTYCQQKS |
| underlined) | | LECLHPGTKFLNNGT |
| | | CTAEGKFSVSLKHGN |
| | | TDSEGIVEVKLVDQD |
| | | KTMFICKSSWSMREA |
| | | NVACLDLGFQQGADT |
| | | QRRFKLSDLSINSTE |
| | | CLHVHCRGLETSLAE |
| | | CTFTKRRTMGYQDFA |
| | | DVVCYTQKADSPMDD |
| | | FFQCVNGKYISQMKA |
| | | CDGINDCGDQSDELC |
| | | CKACQGKGFHCKSGV |
| | | CIPSQYQCNGEVDCI |
| | | TGEDEVGCAGFASVT |
| | | QEETEILTADMDAER |
| | | RRIKSLLPKLSCGVK |
| | | NRMHIRRKRIVGGKR |
| | | AQLGDLPWQVAIKDA |
| | | SGITCGGIYIGGCWI |
| | | LTAAHCLRASKTHRY |
| | | QIWTTVVDWIHPDLK |
| | | RIVIEYVDRIIFHEN |
| | | YNAGTYQNDIALIEM |
| | | KKDGNKKDCELPRSI |
| | | PACVPWSPYLFQPND |
| | | TCIVSGWGREKDNER |
| | | VFSLQWGEVKLISNC |
| | | SKFYGNRFYEKEMEC |
| | | AGTYDGSIDACKGDS |
| | | GGPLVCMDANNVTYV |
| | | WGVVSWGENCGKPEF |
| | | PGVYTKVANYFDWIS |
| | | YHVGRPFISQYNV |
| | | (SEQ ID NO: 1) |
| wt hCFI, | hCFI | KVTYTSQEDLVEKKC |
| no signal | | LAKKYTHLSCDKVFC |
| sequence | | QPWQRCIEGTCVCKL |
| | | PYQCPKNGTAVCATN |
| | | RRSFPTYCQQKSLEC |
| | | LHPGTKFLNNGTCTA |
| | | EGKFSVSLKHGNTDS |
| | | EGIVEVKLVDQDKTM |
| | | FICKSSWSMREANVA |
| | | CLDLGFQQGADTQRR |
| | | FKLSDLSINSTECLH |
| | | VHCRGLETSLAECTF |
| | | TKRRTMGYQDFADVV |
| | | CYTQKADSPMDDFFQ |
| | | CVNGKYISQMKACDG |
| | | INDCGDQSDELCCKA |
| | | CQGKGFHCKSGVCIP |
| | | SQYQCNGEVDCITGE |
| | | DEVGCAGFASVTQEE |
| | | TEILTADMDAERRRI |
| | | KSLLPKLSCGVKNRM |
| | | HIRRKRIVGGKRAQL |
| | | GDLPWQVAIKDASGI |
| | | TCGGIYIGGCWILTA |
| | | AHCLRASKTHRYQIW |
| | | TTVVDWIHPDLKRIV |
| | | IEYVDRIIFHENYNA |
| | | GTYQNDIALIEMKKD |
| | | GNKKDCELPRSIPAC |
| | | VPWSPYLFQPNDTCI |
| | | VSGWGREKDNERVFS |
| | | LQWGEVKLISNCSKF |
| | | YGNRFYEKEMECAGT |
| | | YDGSIDACKGDSGGP |
| | | LVCMDANNVTYVWGV |
| | | VSWGENCGKPEFPGV |
| | | YTKVANYFDWISYHV |
| | | GRPFISQYNV |
| | | (SEQ ID NO: 5) |
| Δ(K1-P305), | AA-chain | KLSCGVKNRMHIRRK |
| deletion of | (CFI- | RIVGGKRAQLGDLPW |
| A-chain of | SPD) | QVAIKDASGITCGGI |
| wt hCFI | | YIGGCWILTAAHCLR |
| | | ASKTHRYQIWTTVVD |
| | | WIHPDLKRIVIEYVD |
| | | RIIFHENYNAGTYQN |
| | | DIALIEMKKDGNKKD |
| | | CELPRSIPACVPWSP |
| | | YLFQPNDTCIVSGWG |
| | | REKDNERVFSLQWGE |
| | | VKLISNCSKFYGNRF |
| | | YEKEMECAGTYDGSI |
| | | DACKGDSGGPLVCMD |
| | | ANNVTYVWGVVSWGE |
| | | NCGKPEFPGVYTKVA |
| | | NYFDWISYHVGRPFI |
| | | SQYNV |
| | | (SEQ ID NO: 12) |
| Wild type | Mouse CFI | MKLAHLSLFLLALHL |
| mouse CFI | (mCFI) | SSSRSPSASDLPQEE |
| (https:// | | LVDQKCLLQKYTHRS |
| www.uniprot.org/ | | CNKVFCQPWQRCIEG |
| uniprot/Q61129) | | TCICKLPYQCPRAGT |
| | | PVCAMNGRSYPTYCH |
| | | QKSFECLHPEIKFSH |
| | | NGTCAAEGKFNVSLI |
| | | YGRTKTEGLVQVKLV |
| | | DQDERMFICKNSWSM |
| | | AEANVACVDLGFPLG |
| | | VRDIQGSFNISGNLH |
| | | INDTECLHVHCRGVE |
| | | TSLAECAFTKRRTEL |
| | | SNGLAGVVCYKQDAD |
| | | FPTSLSFQCVNGKHI |
| | | PQEKACNGVNDCGDQ |
| | | SDELCCKGCRGNASL |
| | | CKSGVCIPDQYKCNG |
| | | EVDCITGEDESRCEE |
| | | DRQQNIPKGLARSAQ |
| | | GEAEIETEETEMLTP |
| | | GMDNERKRIKSLLPK |
| | | LSCGVKRNTHTRRKR |
| | | VIGGKPANVGDYPWQ |
| | | VAIKDGQRITCGGIY |
| | | IGGCWILTAAHCVRP |
| | | SRAHSYQVWTALLDW |
| | | LKPNSQLGIQTVKRV |
| | | IVHEKYNGATFONDI |
| | | ALIEMKMHTGKKECE |
| | | LPNSVPACVPWSPYL |
| | | FQPNDRCIISGWGRG |
| | | KDNQKVYSLRWGEVD |
| | | LIGNCSQFYPDRYYE |
| | | KEMQCAGTRDGSIDA |
| | | CKGDSGGPLVCEDIN |
| | | NVTYVWGIVSWGENC |
| | | GKPEFPGVYTRVANY |
| | | FDWISYHVGRSLVSQ |
| | | HNV |
| | | (SEQ ID NO: 23) |
| wt hCFI + | hCFI-hCFI | KVTYTSQEDLVEKKC |
| GSSGG | fusion | LAKKYTHLSCDKVFC |
| (linker) + | | QPWQRCIEGTCVCKL |
| wt hCFI | | PYQCPKNGTAVCATN |
| | | RRSFPTYCQQKSLEC |
| | | LHPGTKFLNNGTCTA |
| | | EGKFSVSLKHGNTDS |
| | | EGIVEVKLVDQDKTM |
| | | FICKSSWSMREANVA |
| | | CLDLGFQQGADTQRR |
| | | FKLSDLSINSTECLH |
| | | VHCRGLETSLAECTF |
| | | TKRRTMGYQDFADVV |
| | | CYTQKADSPMDDFFQ |
| | | CVNGKYISQMKACDG |
| | | INDCGDQSDELCCKA |
| | | CQGKGFHCKSGVCIP |
| | | SQYQCNGEVDCITGE |
| | | DEVGCAGFASVTQEE |
| | | TEILTADMDAERRRI |
| | | KSLLPKLSCGVKNRM |
| | | HIRRKRIVGGKRAQL |
| | | GDLPWQVAIKDASGI |
| | | TCGGIYIGGCWILTA |
| | | AHCLRASKTHRYQIW |
| | | TTVVDWIHPDLKRIV |
| | | IEYVDRIIFHENYNA |
| | | GTYQNDIALIEMKKD |
| | | GNKKDCELPRSIPAC |
| | | VPWSPYLFQPNDTCI |
| | | VSGWGREKDNERVFS |
| | | LQWGEVKLISNCSKF |
| | | YGNRFYEKEMECAGT |
| | | YDGSIDACKGDSGGP |
| | | LVCMDANNVTYVWGV |
| | | VSWGENCGKPEFPGV |
| | | YTKVANYFDWISYHV |
| | | GRPFISQYNVGSSGG |
| | | KVTYTSQEDLVEKKC |
| | | LAKKYTHLSCDKVFC |
| | | QPWQRCIEGTCVCKL |
| | | PYQCPKNGTAVCATN |
| | | RRSFPTYCQQKSLEC |
| | | LHPGTKFLNNGTCTA |
| | | EGKFSVSLKHGNTDS |
| | | EGIVEVKLVDQDKTM |
| | | FICKSSWSMREANVA |
| | | CLDLGFQQGADTQRR |
| | | FKLSDLSINSTECLH |
| | | VHCRGLETSLAECTF |
| | | TKRRTMGYQDFADVV |
| | | CYTQKADSPMDDFFQ |
| | | CVNGKYISQMKACDG |
| | | INDCGDQSDELCCKA |
| | | CQGKGFHCKSGVCIP |
| | | SQYQCNGEVDCITGE |
| | | DEVGCAGFASVTQEE |
| | | TEILTADMDAERRRI |
| | | KSLLPKLSCGVKNRM |
| | | HIRRKRIVGGKRAQL |
| | | GDLPWQVAIKDASGI |
| | | TCGGIYIGGCWILTA |
| | | AHCLRASKTHRYQIW |
| | | TTVVDWIHPDLKRIV |
| | | IEYVDRIIFHENYNA |
| | | GTYQNDIALIEMKKD |
| | | GNKKDCELPRSIPAC |
| | | VPWSPYLFQPNDTCI |
| | | VSGWGREKDNERVFS |
| | | LQWGEVKLISNCSKF |
| | | YGNRFYEKEMECAGT |
| | | YDGSIDACKGDSGGP |
| | | LVCMDANNVTYVWGV |
| | | VSWGENCGKPEFPGV |
| | | YTKVANYFDWISYHV |
| | | GRPFISQYNV |
| | | (SEQ ID NO: 16) |

In some embodiments, a base molecule itself may be a CFI variant, for example in some embodiments, a CFI variant comprising only the serine protease domain (CFI-SPD) itself is a CFI variant. In some embodiments, the CFI variants are derived from any base molecule of Table 1, and comprise modifications to loops corresponding to the loops of an unmodified CFI. In some embodiments, the CFI variants are derived from any base molecule of Table 1, and comprise substitution mutations. In some embodiments, the CFI variants are derived from any base molecule of Table 1, and comprise a deletion of one or more domains of CFI. In some embodiments, the CFI variants are derived from any base molecule of Table 1, and comprise an inversion of the A-chain and the B-chain of the CFI.

In some embodiments, provided herein are CFI variants comprising at least one CFI domain, wherein the at least one CFI domain corresponds to those of a wild type CFI of any species. For example, the amino acid sequence of the at least one CFI domain may comprise the amino acid sequence derived from a wild type human CFI as set forth in SEQ ID NO: 5. The CFI variants provided herein comprising an amino acid sequence derived from SEQ ID NO: 5 may comprise one or more modifications with respect to the sequence set forth in SEQ ID NO: 5. For example, the one or more modifications may include a deletion of one or more amino acid residues, substitution mutations of one or more amino acid residues, an addition of one or more amino acid residues, the deletion of one or more domains of CFI, the substitution of one or more domains of CFI, or the addition of one or more domains of CFI.

In some embodiments, provided herein are CFI variants comprising at least one CFI domain of any species, wherein the at least one CFI domain comprises any one or more CFI domains selected from: a serine protease domain (SPD), a Factor I membrane attack complex (FIMAC) domain, a scavenger receptor cysteine-rich domain (SRCR), a low density lipoprotein receptor 1 (LDLr1), and low density lipoprotein receptor 2 (LDLr2) domains. In some embodiments, the any one or more CFI domains are that of a human CFI. In some embodiments, the any one or more CFI domains comprise an amino acid sequence derived from the sequence set forth in SEQ ID NO: 5.

In some embodiments, the CFI variants comprise all domains of a wild type CFI, i.e., each one of the SPD, the FIMAC domain, the SRCR domain, the LDLr1 domain, and the LDLr2 domain, and comprises a modification in any one or more of these domains with respect to the wild type CFI.

In some embodiments, the CFI variants do not comprise all of the domains corresponding to that of the wild type CFI. In some embodiments, the CFI variants comprise the SPD. In some embodiments, the CFI variants comprise only the SPD, wherein the A-chain of the CFI has been deleted, referred to herein as “CFI-SPD.” In some embodiments, the CFI-SPD comprises the amino acid sequence set forth in SEQ ID NO: 12 (as shown in Table 1), which is the SPD of a human CFI. In some embodiments, the CFI-SPD comprises no further modifications with respect to that of a wild type CFI SPD. In some embodiments, the CFI-SPD comprises one or more modifications with respect to that of a wild type CFI SPD. In some embodiments, the CFI-SPD comprises at least one modification with respect to the amino acid sequence set forth in SEQ ID NO: 12.

Exemplary variants of CFI are described in further detail below. Exemplary CFI variants comprise one or more substitutions of amino acid residues with respect to a CFI having the amino acid sequence set forth in SEQ ID NO: 5. For example, a CFI variant that includes substitutions at positions S499 and 1500 will have substitutions at positions S499 and 1500 in the amino acid sequence set forth in SEQ ID NO: 5.

### Exemplary CFI Variants

Provided herein are CFI variants comprising or consisting of at least one modification with respect to a wild type CFI, wherein the CFI variant is capable of increasing complement system inhibition, and wherein the CFI variant has at least one improved characteristic as compared to the wild type CFI. Examples of improved characteristic include, but are not limited to, an increase in half-life, an increase in bioavailability or an increase or decrease in any one or more of activity, substrate specificity, potency, substrate affinity, cofactor affinity and catalytic capability. In exemplary embodiments, an improved characteristic is increased half-life. In other exemplary embodiments, an improved characteristic is increased, or altered substrate specificity.

Without limitation, the disclosure contemplates the exemplary CFI variants described in Table 3. The variants of Table 3 include modified CFIs, as well as CFI fusion constructs, described herein. For avoidance of doubt, unless otherwise indicated, where a residue number is indicated, it refers to SEQ ID NO: 5 (wild type human CFI), or a sequence corresponding thereto. For avoidance of doubt, by way of example a variant whose description is P433A is a CFI variant comprising a P433A substitution, e.g. a CFI variant comprising a P433A substitution in SEQ ID NO: 5 (or a sequence corresponding thereto); the disclosure also provides for a CFI variant consisting of a P433A substitution, e.g. a CFI variant, wherein SEQ ID NO: 5 has a P433A substitution.

The CFI variants of the disclosure are provided individually in Table 2. In some embodiments, a CFI variant of the disclosure comprises or consists of any one or more of the modifications presented in Table 2, wherein the positions correspond to positions in a CFI having the amino acid sequence set forth in SEQ ID NO: 5. The construct number makes reference to Table 3, depicting the construct in which the indicated variant is present.

CFI variants of the disclosure may have at least one, at least two, at least three, at least four, at least five, at least six, at least seven, or more modifications, e.g. substitutions, deletions, insertions and fusions. Modification, e.g. substitutions, for a given variant may be represented in one of many ways recognized by the skilled artisan. For example, a hCFI variant having substitutions at D395A and E416A may be referred to as having substitutions: “D395A and E416A”, “D395A-E416A”, “D395A+E416A”. “D395A/E416A”, or “D395A; E416A” and are used interchangeably herein. In some instances, a CFI variant having substitutions at D395A and E416A may be referred to as “hCFI; D395A; E416A” or CFI variant (D395A; E416A).” As described herein, variants with other modifications, such as deletions, or combinations of modifications, such as deletions, fusions and substitutions, can conform to similar styles of nomenclature.

Table 3 provides exemplary CFI variants of the disclosure, each represented by unique construct number (abbreviated as “Const. No.” in Table 3) This and other tables disclosing variants may include the following symbols and abbreviations and associated meanings: HSA=human serum albumin; CFI=complement factor I; Δ=Deletion of the amino acid range noted; →or>=Deletion of noted sequence and replaced with noted amino acids; Cr1=CR1 fusion; Fh=FH fusion; G(#) denotes a linker of glycines repeated the indicated number of instances; (GGSS)ⁿ denotes a linker of GGSS repeated n number of times (SEQ ID NO: 26); (GGSS)ⁿGG denotes a linker of GGSS repeated n number of times followed by GG (SEQ ID NO: 27).

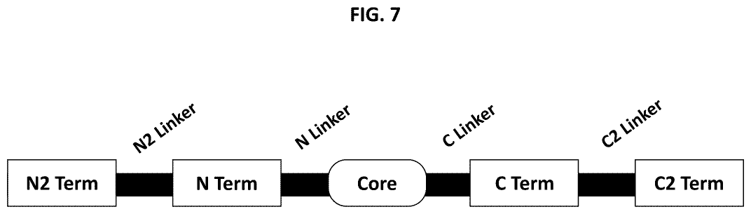

FIG. 7 corresponds to the layout of Table 3, and depicts a schematic representation of an exemplary CFI fusion construct, wherein the “Core” domain comprises any one or more of the amino acid modifications of the disclosure, and may be flanked by one or more domains in either or both the N and C termini. The domains depicted may be optionally connected by linkers.

TABLE 2
CFI Variants

| Const. | |
|---|---|
| No. of | |
| Table 3 | Core |
| 1 | hCFI; F559W |
| 2 | hCFI; F559H |
| 3 | hCFI; L307F |
| 4 | hCFI; L307H |
| 5 | hCFI; L307W |
| 6 | hCFI; L307Y |
| 7 | hCFI; P433A |
| 8 | hCFI; P433G |
| 9 | hCFI; P433F |
| 10 | hCFI; Δ(Y496) |
| 11 | hCFI; Δ(D497) |
| 12 | hCFI; Δ(Y496; D497) |
| 13 | hCFI; S499G |
| 14 | hCFI; S499A |
| 15 | hCFI; S499K |
| 16 | hCFI; S499V |
| 17 | hCFI; Y496L |
| 18 | hCFI; S499G; I500K |
| 19 | hCFI; S499A; I500K |
| 20 | hCFI; T495F; Y496L; S499G; |
| | I500K |
| 21 | hCFI; K423R |
| 22 | hCFI; R62A |
| 23 | hCFI; R62E |
| 24 | hCFI; R62L |
| 25 | hCFI; R62H |
| 26 | hCFI; E401K |
| 27 | hCFI; E401R |
| 28 | hCFI; E401A |
| 29 | hCFI; E401F |
| 30 | hCFI; N402G |
| 31 | hCFI; N402K |
| 32 | hCFI; N402R |
| 33 | hCFI; H554F |
| 34 | hCFI; H554K |
| 35 | hCFI; R371E |
| 36 | hCFI; A342R |
| 37 | hCFI; K51R; N52A; A55P; |
| | T59M; R61G; F64Y |
| 38 | hCFI; N52A |
| 39 | hCFI; A55P |
| 40 | hCFI; T59E |
| 41 | hCFI; L73F |
| 42 | hCFI; L73D |
| 43 | hCFI; Δ(275-CAGFASVAQE- |
| | 284)(SEQ ID NO: 31) > |
| | CEEDRQQNIPKGLARSAQG |
| | EAEIETE (SEQ ID NO: 32) |
| 44 | hCFI; Δ(275-CAGFASVAQE- |
| | 284)(SEQ ID NO: 31) > |
| | CEEDRQQNIPKGLARSAQG |
| | EAEIETE (SEQ ID NO: 32) + |
| | GGSSGG (SEQ ID NO: 6) + |
| | CR1(ccp15-17) |
| 45 | hCFI; R484A |
| 46 | hCFI; R484K |
| 47 | hCFI; R484E |
| 48 | hCFI; Δ(V565) > VG |
| 49 | hCFI; Δ(V565) > VY |
| 50 | hCFI; E416K |
| 51 | hCFI; E416R |
| 52 | hCFI; D395A |
| 53 | hCFI; D395G |
| 54 | hCFI; D395K |
| 55 | hCFI; D395R |
| 56 | hCFI; D395A; D425A |
| 57 | hCFI; D395A; E416A |
| 58 | hCFI; D395A; E416A; D425A |
| 59 | hCFI; N402D |
| 60 | hCFI; K368D |
| 76 | hCFI; H383K; D385N; |
| | K387S; R388Q; E392Q; |
| | Y393T; D395K; K419M; |
| | D420H; G421T; N422G; |
| | D425E |
| 77 | hCFI; H383K; R388Q; |
| | E392Q; Y393T; K419M |
| 78 | hCFI; K51D; T59E; R61D |
| 79 | hCFI; Q69H; L73F; G79E; |
| | T80I; L83S; N84H |
| 80 | hCFI; Δ(1-KVTYTS-6)(SEQ |
| | ID NO: 35) > RSPSASDLP |
| | (SEQ ID NO: 36) |
| 81 | hCFI; M186E; G187L; Y188S; |
| | Q189N; D190G; F191L; |
| | D193G |
| 89 | hCFI; T377G; E457G; N531G |
| 90 | hCFI; T377G; E457G; N531G |
| 91 | hCFI; T377A; E457G; N531G |
| 92 | hCFI; N422A |
| 93 | hCFI; T377A |
| 95 | hCFI |

### Tuning of Activity and Specificity

The activity and specificity of the CFI variants provided herein can be tuned (adjusted) for particular applications and therapeutic indications. For example, activity and specificity can be tuned by selection of C3b degraders, or C4b degraders, or degraders of both C3b and C4b. As referred to herein, protease activity for a substrate refers to the ability of a CFI variant of the disclosure to cleave its substrates, C4b and C3b. This can be expressed in a number of ways, for example as an increase in C4b degrader activity, protease activity towards C4b, C3b degrader activity, protease activity towards C3b, yield of cleavage products, and the like.

As used herein a C3b degrader is a CFI variant that is capable of cleaving C3b; likewise, a C4b degrader is a CFI variant that is capable of cleaving C4b. The use of C3b degrader does not imply that it does not degrade C4b. A CFI variant can be both a C3b degrader, and a C4b degrader, and may, but not necessarily, show specificity for one over the other.

The CFI variants provided herein have modified characteristics that include increases or decreases in protease activity for a substrate as well increases or decreases in substrate specificity.

As used herein, and demonstrated in the examples provided herein, specificity for a substrate, also referred to as substrate specificity, refers to the specificity for one over the other that a CFI variant demonstrates. If a substrate specificity of a CFI variant is about 1, then the specificity for both C4b and C3b are equal relative to wild-type CFI activity. If the specificity of a CFI variant is 2-fold higher for C4b, then it is deemed to demonstrate increased specificity of cleavage for C4b, as compared to C3b. An increase in protease activity for one substrate by a greater fold increase as compared to another substrate is an example of an increase in specificity for that substrate.

In some embodiments amino acids modifications (e.g. substitutions) either increase activity, confer specificity or both. In some embodiments, an increase in C4b degrader activity comprises an increase in the cleavage of C4b, (and the generation of a cleavage product such as C4c) and an increase in the specificity towards C4b comprises an increase in the cleavage of C4b and a decrease in the cleavage of C3b (and the generation of a cleavage product such as iC3b). Generally, the comparison is provided as compared to wild type CFI or a fusion construct comprising wild type CFI.

In some embodiments the combination of two or more modifications (e.g. substitutions) may confer unexpected increases in activity that are synergistic or additive.

In some embodiments the combination of one or more modifications may confer unexpected increases or decreases in activity that are synergistic when C4b is the substrate and additive or less than additive when C3b is the substrate.

In some embodiments the combination of one or more modifications may confer unexpected increases or decreases in activity that are synergistic when C3b is the substrate and additive or less than additive when C4b is the substrate.

In some embodiments, a modified characteristic can be achieved by selection (e.g. tuning) of one or more modifications that confer increased C3b degrader activity and decrease C4b degrader activity (increase in C3b substrate specificity) or, alternatively, confer increased C4b degrader activity and decrease C3b degrader activity (increase in C4b substrate specificity) or, alternatively, provide increased activity as degraders of both C3b and C4b (no change in specificity, but increase in activity for both substrates).

In some embodiments, a modified characteristic can be achieved by selection of one or more modifications that confer increased C3b degrader activity and no change in C4b degrader activity (increase in C3b substrate specificity) or, alternatively, confer increased C4b degrader activity and no change in C3b degrader activity (increase in C4b substrate specificity).

In some embodiments, a modified characteristic can be achieved by selection of one or more modifications that confer a decrease in C3b degrader activity and no change in C4b degrader activity (increase in C4b substrate specificity) or, alternatively, confer a decrease in C4b degrader activity and no change in C3b degrader activity (increase in C3b substrate specificity).

Modifications providing increased activity and specificity are typically concentrated in, but not bound by limitation, to structural regions critical for CFI function. Exemplary structural regions where modifications (e.g. substitutions) that can lead to at least one improved characteristic are the C-terminal extension, the A:B interface, the surface representing an interface with cofactors and modifications (e.g. substitutions) within the active site of the SPD including surface loops that provide an interface with the C3b and C4b substrates and the CR1 and FH cofactors (referring to FIG. 1).

Without being bound to theory or mechanism, provided herein are CFI variants having one or more combinations of any of the amino acid modifications detailed below, wherein the CFI variants have at least one improved characteristic. CFI variants with combined modifications (e.g. substitutions) comprise two or more modifications in one or more regions of CFI selected from, but not limited to the structural regions of the C-terminal extension, the A:B interface, the interface with cofactors and the active site, including surface loops that provide an interface with cofactors and the C3b or C4b substrates.

In some embodiments CFI variants comprising two or more substitutions exhibit changes in activity, substrate specificity, or both. In some embodiments, an increase in activity comprises an increase in the cleavage of C4b, and/or the generation of C4c and specificity comprises a limited increase or a decrease in the cleavage of C3b, and/or the generation of iC3b as compared to wild type CFI (or compared to a fusion construct comprising wild type CFI, e.g. SEQ ID NO: 21). In some embodiments the combination of two or more substitutions confers unexpected increases in activity that are synergistic when C4b is the substrate and additive or less than additive when C3b is the substrate.

In some embodiments amino acids substitutions either increase activity, confer specificity or both and may switch between C3b selectivity and C4b selectivity. In some embodiments, an increase in activity comprises an increase in the cleavage of C4b, and/or the generation of C4c and selectivity comprises a decrease in the cleavage of C3b, and/or the generation of iC3b as compared to wild type CFI. In some embodiments, an increase in activity comprises an increase in the cleavage of C3b, and/or the generation of iC3b and specificity comprises a decrease in the cleavage of C4b, and/or the generation of C4c as compared to wild-type CFI. In some embodiments the nature of the amino acid substitution defines whether the CFI variant displays characteristics of specificity for C3b or specificity for C4b.

Exemplary variants of the disclosure are tested for differences in activity, and for differences in specificity. Exemplary data are provided in Table 7.2.

In some embodiments, the CFI variant exhibits increased activity, wherein the increase in activity comprises an increase in the C3b degrader activity by a CFI variant of the disclosure (with a concomitant increase in a C3b cleavage product). In some embodiments, a CFI variant of the disclosure exhibits increase C3b degrader activity by at least or about 1.5-fold, at least or about 2-fold, at least or about 3-fold, at least or about 4-fold at least or about 5-fold, at least or about 6-fold, at least or about 7-fold at least or about 8-fold, at least or about 9-fold, at least or about 10-fold at least or about 15-fold, at least or about 20-fold, at least or about 25-fold at least or about 30-fold, at least or about 40-fold, at least or about 50-fold at least or about 75-fold, at least or about 100-fold, at least or about 150-fold at least or about 200-fold, at least or about 250-fold, at least or about 300-fold, at least or about 350-fold at least or about 400-fold, at least or about 450-fold, at least or about 500-fold, at least or about 550-fold at least or about 600-fold, at least or about 650-fold, at least or about 700-fold, at least or about 750-fold at least or about 800-fold, at least or about 850-fold, at least or about 900-fold, at least or about 950-fold at least or even at least about 1000-fold, as compared to a wild type CFI, or a fusion construct comprising a wild type CFI. e.g. SEQ ID NO: 21. In some embodiments, this increase in C3b degrader activity is accompanied by an increase also in C4b degrader activity. In some embodiments, this increase in C3b degrader activity is not accompanied by an increase also in C4b degrader activity, and there may even be a decrease in C4b degrader activity.

In some embodiments, the CFI variant exhibits increased activity, wherein the increase in activity comprises an increase in the C4b degrader activity by a CFI variant of the disclosure (with a concomitant increase in a C4b cleavage product). In some embodiments, a CFI variant of the disclosure exhibits increase C4b degrader activity by at least or about 1.5-fold, at least or about 2-fold, at least or about 3-fold, at least or about 4-fold at least or about 5-fold, at least or about 6-fold, at least or about 7-fold at least or about 8-fold, at least or about 9-fold, at least or about 10-fold at least or about 15-fold, at least or about 20-fold, at least or about 25-fold at least or about 30-fold, at least or about 40-fold, at least or about 50-fold at least or about 75-fold, at least or about 100-fold, at least or about 150-fold at least or about 200-fold, at least or about 250-fold, at least or about 300-fold, at least or about 350-fold at least or about 400-fold, at least or about 450-fold, at least or about 500-fold, at least or about 550-fold at least or about 600-fold, at least or about 650-fold, at least or about 700-fold, at least or about 750-fold at least or about 800-fold, at least or about 850-fold, at least or about 900-fold, at least or about 950-fold at least or even at least about 1000-fold, as compared to a wild type CFI, or a fusion construct comprising a wild type CFI. e.g. SEQ ID NO: 21. In some embodiments, this increase in C4b degrader activity is accompanied by an increase also in C3b degrader activity. In some embodiments, this increase in C4b degrader activity is not accompanied by an increase also in C3b degrader activity, and there may even be a decrease in C3b degrader activity.

In some embodiments, the CFI variant exhibits increased activity, wherein the increase in activity comprises an increase in both C3b and C4b degrader activity. In some embodiments, a CFI variant of the disclosure exhibits both increased C3b and increased C4b degrader activity by at least or about 1.5-fold. at least or about 2-fold, at least or about 3-fold, at least or about 4-fold at least or about 5-fold, at least or about 6-fold, at least or about 7-fold at least or about 8-fold, at least or about 9-fold, at least or about 10-fold at least or about 15-fold, at least or about 20-fold, at least or about 25-fold at least or about 30-fold, at least or about 40-fold, at least or about 50-fold at least or about 75-fold, at least or about 100-fold, at least or about 150-fold at least or about 200-fold, at least or about 250-fold, at least or about 300-fold, at least or about 350-fold at least or about 400-fold, at least or about 450-fold, at least or about 500-fold, at least or about 550-fold at least or about 600-fold, at least or about 650-fold, at least or about 700-fold, at least or about 750-fold at least or about 800-fold, at least or about 850-fold, at least or about 900-fold, at least or about 950-fold at least or even at least about 1000-fold, as compared to a wild type CFI, or a fusion construct comprising a wild type CFI, e.g. SEQ ID NO: 21. The increase in degrader activity of one substrate may be the same, but need not be.

In some embodiments, the CFI variant exhibits increased specificity for a substrate, wherein the increase in specificity is for C3b (over C4b). In some embodiments, a CFI variant of the disclosure exhibits increased specificity for C3b by at least or about 1.5-fold, at least or about 2-fold, at least or about 3-fold, at least or about 4-fold at least or about 5-fold, at least or about 6-fold, at least or about 7-fold at least or about 8-fold, at least or about 9-fold, at least or about 10-fold at least or about 15-fold, at least or about 20-fold, at least or about 25-fold at least or about 30-fold, at least or about 40-fold, at least or about 50-fold at least or about 75-fold, at least or about 100-fold, at least or about 150-fold at least or about 200-fold, at least or about 250-fold, at least or about 300-fold, at least or about 350-fold at least or about 400-fold, at least or about 450-fold, at least or about 500-fold, at least or about 550-fold at least or about 600-fold, at least or about 650-fold, at least or about 700-fold, at least or about 750-fold at least or about 800-fold, at least or about 850-fold, at least or about 900-fold, at least or about 950-fold at least or even at least about 1000-fold, as compared to a wild type CFI, or a fusion construct comprising a wild type CFI, e.g. SEQ ID NO: 21.

In some embodiments, the CFI variant exhibits increased specificity for a substrate, wherein the increase in specificity is for C4b (over C3b). In some embodiments, a CFI variant of the disclosure exhibits increased specificity for C4b by at least or about 1.5-fold, at least or about 2-fold, at least or about 3-fold, at least or about 4-fold at least or about 5-fold, at least or about 6-fold, at least or about 7-fold at least or about 8-fold, at least or about 9-fold, at least or about 10-fold at least or about 15-fold, at least or about 20-fold, at least or about 25-fold at least or about 30-fold, at least or about 40-fold, at least or about 50-fold at least or about 75-fold, at least or about 100-fold, at least or about 150-fold at least or about 200-fold, at least or about 250-fold, at least or about 300-fold, at least or about 350-fold at least or about 400-fold, at least or about 450-fold, at least or about 500-fold, at least or about 550-fold at least or about 600-fold, at least or about 650-fold, at least or about 700-fold, at least or about 750-fold at least or about 800-fold, at least or about 850-fold, at least or about 900-fold, at least or about 950-fold at least or even at least about 1000-fold, as compared to a wild type CFI, or a fusion construct comprising a wild type CFI, e.g. SEQ ID NO: 21 which has an about equal specificity for both C3b and C4b.

In some embodiments, the CFI variant exhibits decreased specificity for a substrate, wherein the decrease in specificity is for C3b (over C4b). In some embodiments, a CFI variant of the disclosure exhibits decreased specificity for C3b by at least or about 1.5-fold, at least or about 2-fold, at least or about 3-fold, at least or about 4-fold at least or about 5-fold, at least or about 6-fold, at least or about 7-fold at least or about 8-fold, at least or about 9-fold, at least or about 10-fold at least or about 15-fold, at least or about 20-fold, at least or about 25-fold at least or about 30-fold, at least or about 40-fold, at least or about 50-fold at least or about 75-fold, at least or about 100-fold, at least or about 150-fold at least or about 200-fold, at least or about 250-fold, at least or about 300-fold, at least or about 350-fold at least or about 400-fold, at least or about 450-fold, at least or about 500-fold, at least or about 550-fold at least or about 600-fold, at least or about 650-fold, at least or about 700-fold, at least or about 750-fold at least or about 800-fold, at least or about 850-fold, at least or about 900-fold, at least or about 950-fold at least or even at least about 1000-fold, as compared to a wild type CFI, or a fusion construct comprising a wild type CFI, e.g. SEQ ID NO: 21 which has an about equal specificity for both C3b and C4b.

In some embodiments, the CFI variant exhibits decreased specificity for a substrate, wherein the decrease in specificity is for C4b (over C3b). In some embodiments, a CFI variant of the disclosure exhibits decreased specificity for C4b by at least or about 1.5-fold, at least or about 2-fold, at least or about 3-fold, at least or about 4-fold at least or about 5-fold, at least or about 6-fold, at least or about 7-fold at least or about 8-fold, at least or about 9-fold, at least or about 10-fold at least or about 15-fold, at least or about 20-fold, at least or about 25-fold at least or about 30-fold, at least or about 40-fold, at least or about 50-fold at least or about 75-fold, at least or about 100-fold, at least or about 150-fold at least or about 200-fold, at least or about 250-fold, at least or about 300-fold, at least or about 350-fold at least or about 400-fold, at least or about 450-fold, at least or about 500-fold, at least or about 550-fold at least or about 600-fold, at least or about 650-fold, at least or about 700-fold, at least or about 750-fold at least or about 800-fold, at least or about 850-fold, at least or about 900-fold, at least or about 950-fold at least or even at least about 1000-fold, as compared to a wild type CFI, or a fusion construct comprising a wild type CFI, e.g. SEQ ID NO: 21 which has an about equal specificity for both C3b and C4b.

In some embodiments, the CFI variant having has increased activity, wherein in the increased activity comprises increased cleavage of C4b and/or specificity for C4b over C3b. In some embodiments, the CFI variant having an increase in the cleavage of C4b comprises one or more substitutions in an amino acid positions set forth in SEQ ID NO: 5. In some embodiments, the amino acid position is a position within the A:B interface region in a CFI having the amino acid sequence set forth in SEQ ID NO: 5. In some embodiments, the amino acid position is a position within the cofactor interface region in a CFI having the amino acid sequence set forth in SEQ ID NO: 5. In some embodiments, the amino acid position is a position within the C-terminal extension region in a CFI having the amino acid sequence set forth in SEQ ID NO: 5.In some embodiments, the amino acid position is a position within the c-terminal extension; C4b interface region in a CFI having the amino acid sequence set forth in SEQ ID NO: 5.In some embodiments, the amino acid position is a position within the C4b interface region in a CFI having the amino acid sequence set forth in SEQ ID NO: 5.In some embodiments, the amino acid position is a position within the active site; C4b interface region in a CFI having the amino acid sequence set forth in SEQ ID NO: 5. In some embodiments, the amino acid position is a position within the autolysis loop: cofactor interface region in a CFI having the amino acid sequence set forth in SEQ ID NO: 5. In some embodiments, the amino acid position is a position within the active site; S1 entrance frame region in a CFI having the amino acid sequence set forth in SEQ ID NO: 5. In some embodiments, the amino acid position is a position within the S1 entrance frame region in a CFI having the amino acid sequence set forth in SEQ ID NO: 5. These differences are as compared to wild type CFI (or compared to a fusion construct comprising wild type CFI, e.g. SEQ ID NO: 21), wherein the positions correspond to positions in a CFI having the amino acid sequence set forth in SEQ ID NO: 5.

In some embodiments, the improved characteristic is an increase in activity, wherein the increase in activity comprises an increase in the cleavage of C3b and/or C4b. In some embodiments, the CFI variants provided herein are C3b degraders, referring to the ability of the CFI variants to increase C3b cleavage. In some embodiments, the CFI variants provided herein are C4b degraders, referring to the ability of the CFI variants to increase C4b cleavage. In some embodiments, the CFI variants provided herein are C3b and C4b degraders, referring to the ability of the CFI variants to increase cleavage of both C3b and C4b.

In some embodiments, the CFI variant having has increased activity, wherein the increased activity comprises increased cleavage of C3b and C4b. In some embodiments, the CFI variant having an increase in the cleavage of C3b and C4b comprises one or more substitutions in amino acid positions set forth in SEQ ID NO: 5. In some embodiments, the amino acid position is a position within the substrate interface region in a CFI having the amino acid sequence set forth in SEQ ID NO: 5. In some embodiments, the position within the substrate interface region is E401. In some embodiments, the amino acid position is a position within the active site; substrate interface region in a CFI having the amino acid sequence set forth in SEQ ID NO: 5. In some embodiments, the amino acid position is a position within the cofactor interface region in a CFI having the amino acid sequence set forth in SEQ ID NO: 5. In some embodiments, the amino acid position is a position within the C-terminal extension region in a CFI having the amino acid sequence set forth in SEQ ID NO: 5. In some embodiments, the amino acid position is a position within the autolysis loop; cofactor interface region in a CFI having the amino acid sequence set forth in SEQ ID NO: 5. In some embodiments, the amino acid position is a position within the active site; S1 entrance frame region in a CFI having the amino acid sequence set forth in SEQ ID NO: 5. These differences are as compared to wild type CFI (or compared to a fusion construct comprising wild type CFI, e.g. SEQ ID NO: 21), wherein the positions correspond to positions in a CFI having the amino acid sequence set forth in SEQ ID NO: 5.

In some embodiments, the CFI variant having has increased activity, wherein the increase in activity comprises an increase in the cleavage of C3b by a CFI variant of the disclosure and does not comprise or minimally comprises an increase in the cleavage of C4b. In some embodiments, the amino acid position is a position within the active site; C3b interface region in a CFI having the amino acid sequence set forth in SEQ ID NO: 5. In some embodiments, the amino acid position is a position within the cofactor interface region in a CFI having the amino acid sequence set forth in SEQ ID NO: 5. In some embodiments, the amino acid position is a position within the c-terminal extension region in a CFI having the amino acid sequence set forth in SEQ ID NO: 5. These differences are as compared to wild type CFI (or compared to a fusion construct comprising wild type CFI, e.g. SEQ ID NO: 21), wherein the positions correspond to positions in a CFI having the amino acid sequence set forth in SEQ ID NO: 5.

In some embodiments, the CFI variant has increased activity, wherein the increase in activity comprises an increase in the cleavage of C4b by a CFI variant of the disclosure and does not comprise or minimally comprises an increase in the cleavage of C3b. In some embodiments, the CFI variant having an increase in the cleavage of C4b and does not comprise or minimally comprises an increase in the cleavage of C3b comprises one or more substitutions in amino acid positions set forth in SEQ ID NO: 5. In some embodiments, the amino acid position is a position within the A:B interface region in a CFI having the amino acid sequence set forth in SEQ ID NO: 5. In some embodiments, the amino acid position is a position within the cofactor interface region in a CFI having the amino acid sequence set forth in SEQ ID NO: 5. In some embodiments, the amino acid position is a position within the S1 entrance frame region in a CFI having the amino acid sequence set forth in SEQ ID NO: 5. In some embodiments, the amino acid position is a position within the c-terminal extension; C4b interface region in a CFI having the amino acid sequence set forth in SEQ ID NO: 5. In some embodiments, the amino acid position is a position within the C-terminal extension region in a CFI having the amino acid sequence set forth in SEQ ID NO: 5. These differences are as compared to wild type CFI (or compared to a fusion construct comprising wild type CFI, e.g. SEQ ID NO: 21), wherein the positions correspond to positions in a CFI having the amino acid sequence set forth in SEQ ID NO: 5.

In some embodiments, the CFI variants of the disclosure that are specific C3b degraders are useful for the treatment of diseases.

In some embodiments, the CFI variants of the disclosure that are specific C4b degraders are useful for the treatment of diseases.

In some embodiments, the CFI variants of the disclosure that are both C4b and C3b degraders, and show an improved characteristic as compared to wild type CFI (e.g. increased activity for both C4b and C3b) are useful for the treatment of diseases.

For example, the diseases that may be treated by use of the C4b degraders include, but are not limited to a non-ocular condition. In some embodiments, the non-ocular condition is a systemic chronic indication. In some embodiments, the non-ocular condition is a systemic chronic indication selected from the group consisting of: Alzheimer's disease, Amyotrophic lateral sclerosis (ALS), anti-neutrophil cytoplasmic antibody (ANCA)-associated vasculitis, antiphospholipid syndrome, asthma, atherosclerosis, atypical hemolytic uremic syndrome (aHUS), autoimmune hemolytic anemia, bullous pemphigoid (BP), C3 glomerulopathy, chronic kidney failure, chronic obstructive pulmonary disease (COPD), Cold agglutinin disease (CAD), Crohn's disease, diabetic neuropathy, generalized myasthenia gravis (gMG), Granulomatosis with Polyangiitis (GPA), Guillain-Barré Syndrome (GBS), hereditary angioedema (HAE), hidradenitis suppurativa (HS), IgA nephropathy (IgAN), lupus nephritis (LN), membranous glomerulonephritis (MN), microscopic polyangiitis (MPA), motor neuron disease, multifocal motor neuropathy (MMN), multiple sclerosis (MS), non-insulin dependent diabetes, osteoarthritis, pancreatitis, Parkinson's disease, paroxysmal nocturnal hemoglobinuria (PNH), post-transplant lymphoproliferative disease, protein losing enteropathy, psoriasis, pyoderma gangrenosum, rheumatoid arthritis, schizophrenia (SZ), systemic lupus erythematosus (SLE), immune thrombocytopenia (ITP), warm Autoimmune hemolytic anemia (wAIHA), Immune-Complex Membranoproliferative Glomerulonephritis (IC-MPGN), and ulcerative colitis, Lampert-Eaton myasthenic syndrome (LEMS), CHAPLE syndrome (CD55 deficiency), thrombotic microangiography (TMA) and chronic inflammatory demyelinating polyneuropathy (CIDP), Huntington disease and ischemia reperfusion injuries.

In some embodiments, the CFI variants provided here are degraders of both C3b and C4b and are useful for the treatment of diseases.

In some embodiments, an increase in activity comprises an increase in the generation of C3dg and/or C3c from iC3b. Exemplary variants that exhibit such improved characteristics are provided in Table 3, and discussed in the examples.

In some embodiments, an increase in activity comprises a reduction in the levels of C3b α-chain. Exemplary variants that exhibit such improved characteristics are provided in Table 3, and discussed in the examples.

In some embodiments, an increase in activity comprises hydrolysis of a peptide substrate or proteolysis of a macromolecular protein substrate. In some embodiments, the macromolecular protein substrate is C3b. In some embodiments, the macromolecular protein substrate is C4b. In some embodiments, the peptide substrate is a chromogenic substrate, e.g. such peptide substrates are useful in an assay format. Exemplary variants that exhibit such improved characteristics are provided in Table 3, and discussed in the examples.

In some embodiments, an increase in activity comprises a reduction in the levels or function of membrane attack complex (MAC). In some embodiments, a reduction or even inhibition of hemolysis is correlated with the reducing in the levels of MAC, and accordingly, in some embodiments, an increase in activity comprises a decrease (partial or complete) in the observed hemolysis.

In some embodiments, an increase in activity comprises a reduction in the amplification of the complement system for the production of C3b. Exemplary variants that exhibit such improved characteristics are provided in Table 3, and discussed in the examples.

In some embodiments, the CFI variants are sialylated. In some embodiments, the CFI variants are further sialylated as compared to a wild type CFI. In some embodiments the CFI variants are sialylated by in vitro methods post-translationally.

In some embodiments, the CFI variants are activated variants (i.e. in an active two chain form). In some embodiments, the CFI variants are activated by furin (the term furin is inclusive of furin variants). In some embodiments, the CFI variants are activated by furin during production in a host cell. In some embodiments, the activation by furin during production in a host cell is achieved by overexpression of furin, e.g. by stable or transient transfection. In some embodiments, the CFI variant is activated by furin after production and secretion by a host cell, i.e. post-translationally.

References to modifications, such as substitutions, in the following sections are modifications with respect to the amino acid sequence of human CFI as set forth in SEQ ID NO: 5. However, it should be understood that modifications to corresponding amino acid residues of any non-human species may also be made.

### A:B Chain Interface CFI Variants

Provided herein are CFI variants comprising one or more modifications at the interface of the heavy and light chains, also referred to as the A:B chain interface, and variants that cause a disruption to the A:B chain interface.

Without being bound to theory or mechanism, the serine protease domain (SPD) of CFI is thought to be kept in a zymogen-like state, via numerous interactions with its own A-chain. Although naturally occurring CFI can cleave peptide or protein substrates at a relatively slow rate, the rate of cleavage by CFI is increased by disrupting the A:B chain interface.

### C-Terminal Region Variants

In the complex formed between CFI and C3b, the C-terminal extension region is positioned in a cavity between the A and B chain of the bound and slightly twisted CFI molecule. This suggests that the C-terminal extension of CFI could be an important regulatory region for the activation of CFI upon binding to C3b.

### N-Linked Glycosylation Site Variants

Provided herein are CFI variants comprising at least one CFI domain, wherein the at least one CFI domain comprises one or more modifications at N-linked glycosylation sites of CFI.

In some embodiments, the modification at the N-linked glycosylation site is a removal of one or more N-linked glycosylation sites of a CFI.

### Serine Protease Domain Variants

Provided herein are CFI variants comprise or consist of at least one CFI domain, wherein the at least one CFI domain is the serine protease domain (SPD) of CFI, and wherein the CFI variant comprises one or more modifications at the SPD.

In the crystal structure of free CFI, cleavage of the activation loop did not result in the insertion of the newly formed N-terminal (Ile322), which is the next step in the classical activation of serine proteases. Instead, the crystal structure suggests that the C-terminal region of the cleaved activation loop remains in a tightly bent loop structure on the surface of CFI, in the same area that the uncleaved activation loop would have remained. This prevents the insertion into the activation pocket, and thus, maturation of the active site (referred to as classical serine protease activation via induced conformational rearrangements). Upon proteolytic activation of the SPD of CFI the new N-terminus of the activation loop is generally released and inserted into the activation pocket such that the cleaved activation loop forces a full activation of CFI in solution. Thus, mutations in the C-terminal region of the activation loop should not affect cleavage by furin, as the region is beyond the 3′ positions relative to the scissile bond.

Accordingly, provided herein are SPD CFI variants. In some embodiments, the CFI variants comprising one or more modifications within regions of the SPD of CFI (FIG. 1) may comprise one or more modifications at any one or more of the activation loop (residues 322-326 of SEQ ID NO: 5), 37-Loop (residues 342-344 of SEQ ID NO: 5), 60-Loop (residues 366-372 of SEQ ID NO: 5), 70-Loop (residues 377-389 of SEQ ID NO: 5), 99-Loop (residues 403-410 of SEQ ID NO: 5), 110-Loop (residues 418-426 of SEQ ID NO: 5), 150-Autolysis Loop (residues 455-463 of SEQ ID NO: 5), 180-Loop Oxyanion Stabilizing (residues 494-509 of SEQ ID NO: 5), and/or 220-Loop S1 Entrance Frame (residues 529-536 of SEQ ID NO: 5). Specifically, provided herein are CFI variants comprising the SPD of CFI, and wherein the CFI variant comprises any one or more of the modifications presented in Table 5. Table 5 presents exemplary CFI variants comprising one or more modifications to the amino acid sequence set forth in SEQ ID NO: 5, wherein the one or more modifications are in the SPD. The base molecule for the CFI variants presented in Table 5 may be a wild type, human CFI, or a CFI-SPD, wherein the SPD corresponds to that of a wild type CFI (also referred to as Δ(K1-P305) or an A-chain deletion), or a CFI fused with another complement regulator such as Factor H (FH-CFI) or CR1 (CR1-CFI) which are discussed in further detail herein when referring to fusion constructs.

In some embodiments, the CFI variants comprise an autolysis loop substitution. The autolysis loop of serine proteases is part of the activation domain and are involved in substrate specificity. Trypsin has a longer autolysis loop than CFI, and several key residues are unique between the autolysis loops of trypsin and CFI. Differences may also occur between the autolysis loops from different species, such as between mouse and human. The mouse CFI autolysis loop may include a large number of differences as compared to the CFI autolysis loop of human CFI. Exemplary CFI variants may include a CFI variant wherein the autolysis loop of human CFI is swapped with that of human trypsin or swapped with that of mouse CFI. Such autolysis loop variants may help to identify critical residues that are involved in C3b and/or C4b cleavage activity. Accordingly, in some embodiments, provided herein are CFI variants, wherein the CFI variant is a chimera comprising one or more domains from a human CFI, and wherein the human CFI further comprises a substitution of one or more amino acid residues for amino acid residues of a corresponding region from a non-human species CFI. In some embodiments, the non-human species CFI is mouse CFI. Provided also herein are CFI variants wherein the CFI variant is a chimera, and wherein the modification comprises the substitution of one or more amino acid residues of the CFI with amino acid residues from a corresponding region of a non-CFI serine protease. In some embodiments, the non-CFI serine protease is trypsin.

An exemplary autolysis loop CFI variant includes a trypsin autolysis loop substitution, comprising a substitution of an autolysis loop of the CFI (REKDNERVFS, SEQ ID NO: 9) for an autolysis loop of trypsin (NTASSGADYPDE, SEQ ID NO: 10), wherein the autolysis loop occurs between positions corresponding to position 456 and position 465 in a CFI having the amino acid sequence set forth in SEQ ID NO: 5.

Another exemplary autolysis loop CFI variant includes a mouse CFI autolysis loop swap, wherein ⁴⁵⁶REKDNERVES⁴⁶⁵ (SEQ ID NO: 9) swapped to RGKDNQKVYS (SEQ ID NO: 11), wherein the autolysis loop occurs between positions corresponding to position 456 and position 465 in a CFI having the amino acid sequence set forth in SEQ ID NO: 5.

### Active Site Variants

Provided herein are CFI variants comprising or consisting of one or more modifications at the active site of CFI. In some embodiments, provided herein are CFI variants comprising at least one CFI domain, wherein the at least one CFI domain comprises a modification to the amino acid sequence set forth in SEQ ID NO: 5, wherein the modification is at the active site of CFI. In some embodiments, the active site CFI variants may improve the catalytic potential of CFI. In some embodiments, the CFI active site variants may improve the catalytic potential of CFI by improving the active site (catalytic machinery) without affecting C3b or C4b binding or binding specificity, which is dominated by exosite and A-chain interactions.

### A-B Chain Inversion CFI Variants

Provided herein are CFI variants, wherein the CFI comprises an A chain and a B chain, and comprise an inversion of the A chain and the B chain. In some embodiments, the CFI variants without a chain inversion (the individual chains optionally comprising one or more modifications) comprise a structural arrangement from N-terminus to C-terminus, or C-terminus to N-terminus, as (A chain)-(optional linker)-(B chain). In some embodiments, the CFI variants comprise an inversion of the A chain and the B chain (the individual chains optionally comprising one or more modifications), such that the structural arrangement from N-terminus to C-terminus, or C-terminus to N-terminus, is (B chain)-(optional linker)-(A chain). The optional linkers may be of any suitable length, e.g. of at least one amino acid. A linker may be a flexible linker, and may be a peptide of about 1 to about 20 amino acid residues in length, wherein the amino acid residues may comprise glycine residues. The linker may also optionally comprise serine residues. Exemplary flexible linkers can include, but are not limited to, glycine polymers, glycine-serine polymers, glycine-alanine polymers, alanine-serine polymers, or any other suitable flexible linkers known in the art. Exemplary linkers are (GGSS)ⁿ (SEQ ID NO: 28), (GGSSGG)ⁿ (SEQ ID NO: 29), and (GGSS)ⁿ GG (SEQ ID NO: 30) wherein n is any number from about 1 to about 2. Exemplary linkers can be of 1-50, 5-50, 10-50, 15-50, 20-50, 25-50, 1-20, 2-20, 3-20, 4-20, 5-20, 6-20, 7-20, 8-20, 9-20, 10-20, 3-15. 3-10, 3-9, 3-8, 3-7, 3-6, 3-5, 4-15, 4-10, 4-9, 4-8, 4-7, 4-6, 4-5, 5-15, 5-10, 5-9, 5-8, 5-7, 5-6, 6-15, 6-10, 6-9, 6-8, or 6-7 amino acids in length.

Without being bound by theory or mechanism, exemplary CFI variants comprising an inversion of the A and B chains may comprise the amino acid sequences set forth in SEQ ID NOs: 17, 18, 19, or 20. The chains may be held together by optional linkers. The linkers between the A chain and the B chain of the inversion variants may be of any suitable length of at least one amino acid. A linker may be a flexible linker and may be a peptide of about 1 to about 10, 3-11 to about 20 or 1 to about 40 acid residues in length, wherein the amino acid residues may comprise glycine residues. The linker may also optionally comprise serine residues. Exemplary flexible linkers can include, but are not limited to, glycine polymers, glycine-serine polymers, glycine-alanine polymers, alanine-serine polymers, or any other suitable flexible linkers known in the art.

### Additional CFI Variants—Useful For Modulation and or Evaluation of the Complement System

In some embodiments, there are CFI variants provided that, while useful for modulation of the complement system, may also be useful for evaluation of activity of the complement system, e.g. can be considered tool proteins, in addition to having therapeutic value.

For example, these other CFI variants may allow for various tests using the CFI fusion constructs. An exemplary such CFI variant may be non-activatable to serve as a control. Another exemplary such CFI variant may provide an easier activation of a fusion construct.

In some embodiments, such additional CFI variants provided herein comprise a modification to the amino acid sequence set forth in SEQ ID NO: 5. In some embodiments, the envisioned CFI variants provided herein are derived from a wild type mouse CFI. In some embodiments, the envisioned CFI variants provided herein are derived from a wild type human CFI. In some embodiments, the envisioned CFI variants provided herein are further derived from a CFI-SPD.

Exemplary CFI variants may include a non-activatable CFI variant, which may provide certain utilities, e.g. as a decoy, or a control.

### CFI Combination Variants

Provided herein are CFI variants comprising or consisting of two or more modifications with respect to a wild type CFI. The modifications occur in the same or different domains of CFI. In some embodiments, the modifications include two or more substitutions. In some embodiments, the modifications include a substitution and a deletion. In some embodiments, the modifications include a substitution and an addition. In some embodiments, the modifications include a deletion and an addition. In some embodiments, the modifications include a substitution, a deletion, and an addition. As used herein, such variants collectively may be referred to as CFI combination variants.

### B. Fusion Constructs Comprising Complement Factor I

Provided herein are fusion constructs comprising at least a first component (CFI portion) comprising at least one domain of complement factor I, and at least a second component, wherein the first component and second and subsequent components are fused (e.g. contiguous or separated by an optional linker). These fusion constructs are referred to herein as “CFI fusion constructs” or simply as “fusion constructs.” In some embodiments, the fusion construct comprises additional components, e.g. a third component, a fourth component, a fifth component, etc. An exemplary construct of FIG. 7 show two N-terminal components, two C-terminal components, and a core component-that is, a fusion construct with at least five components.

FIG. 7 depicts a schematic representation of an exemplary CFI fusion construct, wherein the “Core” domain comprises any one or more of the CFI variants of the disclosure, and may be flanked by one or more domains (one or more components) in either or both the N and C termini. The domains depicted may be optionally connected by linkers. For example picture in FIG. 7 are two N-terminal components, and two C-terminal components, depicting a five-component fusion construct. The variants provided in Table 3 follow this format. Exemplary first component CFI core domains are provided in Table 2 and below in Table 3.

TABLE 3
Exemplary CFI Variants and Constructs

| Const. | N2 | N2 | N | N | | C | C | C2 | C2 |
|---|---|---|---|---|---|---|---|---|---|
| No. | Term | Linker | Term | Linker | Core | Linker | Term | Linker | Term |
| 1 | | | HSA | GGSS | hCFI; | | | | |
| | | | | GG | F559W | | | | |
| | | | | (SEQ | | | | | |
| | | | | ID | | | | | |
| | | | | NO: | | | | | |
| | | | | 6) | | | | | |
| 2 | | | HSA | GGSS | hCFI; | | | | |
| | | | | GG | F559H | | | | |
| | | | | (SEQ | | | | | |
| | | | | ID | | | | | |
| | | | | NO: | | | | | |
| | | | | 6) | | | | | |
| 3 | | | HSA | GGSS | hCFI; | | | | |
| | | | | GG | L307F | | | | |
| | | | | (SEQ | | | | | |
| | | | | ID | | | | | |
| | | | | NO: | | | | | |
| | | | | 6) | | | | | |
| 4 | | | HSA | GGSS | hCFI; | | | | |
| | | | | GG | L307H | | | | |
| | | | | (SEQ | | | | | |
| | | | | ID | | | | | |
| | | | | NO: | | | | | |
| | | | | 6) | | | | | |
| 5 | | | HSA | GGSS | hCFI; | | | | |
| | | | | GG | L307W | | | | |
| | | | | (SEQ | | | | | |
| | | | | ID | | | | | |
| | | | | NO: | | | | | |
| | | | | 6) | | | | | |
| 6 | | | HSA | GGSS | hCFI; | | | | |
| | | | | GG | L307Y | | | | |
| | | | | (SEQ | | | | | |
| | | | | ID | | | | | |
| | | | | NO: | | | | | |
| | | | | 6) | | | | | |
| 7 | | | HSA | GGSS | hCFI; | | | | |
| | | | | GG | P433A | | | | |
| | | | | (SEQ | | | | | |
| | | | | ID | | | | | |
| | | | | NO: | | | | | |
| | | | | 6) | | | | | |
| 8 | | | HSA | GGSS | hCFI; | | | | |
| | | | | GG | P433G | | | | |
| | | | | (SEQ | | | | | |
| | | | | ID | | | | | |
| | | | | NO: | | | | | |
| | | | | 6) | | | | | |
| 9 | | | HSA | GGSS | hCFI; | | | | |
| | | | | GG | P433F | | | | |
| | | | | (SEQ | | | | | |
| | | | | ID | | | | | |
| | | | | NO: | | | | | |
| | | | | 6) | | | | | |
| 10 | | | HSA | GGSS | hCFI; | | | | |
| | | | | GG | Δ(Y496) | | | | |
| | | | | (SEQ | | | | | |
| | | | | ID | | | | | |
| | | | | NO: | | | | | |
| | | | | 6) | | | | | |
| 11 | | | HSA | GGSS | hCFI; | | | | |
| | | | | GG | Δ(D497) | | | | |
| | | | | (SEQ | | | | | |
| | | | | ID | | | | | |
| | | | | NO: | | | | | |
| | | | | 6) | | | | | |
| 12 | | | HSA | GGSS | hCFI; | | | | |
| | | | | GG | Δ(Y496; | | | | |
| | | | | (SEQ | D497) | | | | |
| | | | | ID | | | | | |
| | | | | NO: | | | | | |
| | | | | 6) | | | | | |
| 13 | | | HSA | GGSS | hCFI; | | | | |
| | | | | GG | S499G | | | | |
| | | | | (SEQ | | | | | |
| | | | | ID | | | | | |
| | | | | NO: | | | | | |
| | | | | 6) | | | | | |
| 14 | | | HSA | GGSS | hCFI; | | | | |
| | | | | GG | S499A | | | | |
| | | | | (SEQ | | | | | |
| | | | | ID | | | | | |
| | | | | NO: | | | | | |
| | | | | 6) | | | | | |
| 15 | | | HSA | GGSS | hCFI; | | | | |
| | | | | GG | S499K | | | | |
| | | | | (SEQ | | | | | |
| | | | | ID | | | | | |
| | | | | NO: | | | | | |
| | | | | 6) | | | | | |
| 16 | | | HSA | GGSS | hCFI; | | | | |
| | | | | GG | S499V | | | | |
| | | | | (SEQ | | | | | |
| | | | | ID | | | | | |
| | | | | NO: | | | | | |
| | | | | 6) | | | | | |
| 17 | | | HSA | GGSS | hCFI; | | | | |
| | | | | GG | Y496L | | | | |
| | | | | (SEQ | | | | | |
| | | | | ID | | | | | |
| | | | | NO: | | | | | |
| | | | | 6) | | | | | |
| 18 | | | HSA | GGSS | hCFI; | | | | |
| | | | | GG | S499G; | | | | |
| | | | | (SEQ | I500K | | | | |
| | | | | ID | | | | | |
| | | | | NO: | | | | | |
| | | | | 6) | | | | | |
| 19 | | | HSA | GGSS | hCFI; | | | | |
| | | | | GG | S499A; | | | | |
| | | | | (SEQ | I500K | | | | |
| | | | | ID | | | | | |
| | | | | NO: | | | | | |
| | | | | 6) | | | | | |
| 20 | | | HSA | GGSS | hCFI; | | | | |
| | | | | GG | T495F; | | | | |
| | | | | (SEQ | Y496L; | | | | |
| | | | | ID | S499G; | | | | |
| | | | | NO: | I500K | | | | |
| | | | | 6) | | | | | |
| 21 | | | HSA | GGSS | hCFI; | | | | |
| | | | | GG | K423R | | | | |
| | | | | (SEQ | | | | | |
| | | | | ID | | | | | |
| | | | | NO: | | | | | |
| | | | | 6) | | | | | |
| 22 | | | HSA | GGSS | hCFI; | | | | |
| | | | | GG | R62A | | | | |
| | | | | (SEQ | | | | | |
| | | | | ID | | | | | |
| | | | | NO: | | | | | |
| | | | | 6) | | | | | |
| 23 | | | HSA | GGSS | hCFI; | | | | |
| | | | | GG | R62E | | | | |
| | | | | (SEQ | | | | | |
| | | | | ID | | | | | |
| | | | | NO: | | | | | |
| | | | | 6) | | | | | |
| 24 | | | HSA | GGSS | hCFI; | | | | |
| | | | | GG | R62L | | | | |
| | | | | (SEQ | | | | | |
| | | | | ID | | | | | |
| | | | | NO: | | | | | |
| | | | | 6) | | | | | |
| 25 | | | HSA | GGSS | hCFI; | | | | |
| | | | | GG | R62H | | | | |
| | | | | (SEQ | | | | | |
| | | | | ID | | | | | |
| | | | | NO: | | | | | |
| | | | | 6) | | | | | |
| 26 | | | HSA | GGSS | hCFI; | | | | |
| | | | | GG | E401K | | | | |
| | | | | (SEQ | | | | | |
| | | | | ID | | | | | |
| | | | | NO: | | | | | |
| | | | | 6) | | | | | |
| 27 | | | HSA | GGSS | hCFI; | | | | |
| | | | | GG | E401R | | | | |
| | | | | (SEQ | | | | | |
| | | | | ID | | | | | |
| | | | | NO: | | | | | |
| | | | | 6) | | | | | |
| 28 | | | HSA | GGSS | hCFI; | | | | |
| | | | | GG | E401A | | | | |
| | | | | (SEQ | | | | | |
| | | | | ID | | | | | |
| | | | | NO: | | | | | |
| | | | | 6) | | | | | |
| 29 | | | HSA | GGSS | hCFI; | | | | |
| | | | | GG | E401F | | | | |
| | | | | (SEQ | | | | | |
| | | | | ID | | | | | |
| | | | | NO: | | | | | |
| | | | | 6) | | | | | |
| 30 | | | HSA | GGSS | hCFI; | | | | |
| | | | | GG | N402G | | | | |
| | | | | (SEQ | | | | | |
| | | | | ID | | | | | |
| | | | | NO: | | | | | |
| | | | | 6) | | | | | |
| 31 | | | HSA | GGSS | hCFI; | | | | |
| | | | | GG | N402K | | | | |
| | | | | (SEQ | | | | | |
| | | | | ID | | | | | |
| | | | | NO: | | | | | |
| | | | | 6) | | | | | |
| 32 | | | HSA | GGSS | hCFI; | | | | |
| | | | | GG | N402R | | | | |
| | | | | (SEQ | | | | | |
| | | | | ID | | | | | |
| | | | | NO: | | | | | |
| | | | | 6) | | | | | |
| 33 | | | HSA | GGSS | hCFI; | | | | |
| | | | | GG | H554F | | | | |
| | | | | (SEQ | | | | | |
| | | | | ID | | | | | |
| | | | | NO: | | | | | |
| | | | | 6) | | | | | |
| 34 | | | HSA | GGSS | hCFI; | | | | |
| | | | | GG | H554K | | | | |
| | | | | (SEQ | | | | | |
| | | | | ID | | | | | |
| | | | | NO: | | | | | |
| | | | | 6) | | | | | |
| 35 | | | HSA | GGSS | hCFI; | | | | |
| | | | | GG | R371E | | | | |
| | | | | (SEQ | | | | | |
| | | | | ID | | | | | |
| | | | | NO: | | | | | |
| | | | | 6) | | | | | |
| 36 | | | HSA | GGSS | hCFI; | | | | |
| | | | | GG | A342R | | | | |
| | | | | (SEQ | | | | | |
| | | | | ID | | | | | |
| | | | | NO: | | | | | |
| | | | | 6) | | | | | |
| 37 | | | HSA | GGSS | hCFI; | | | | |
| | | | | GG | K51R; | | | | |
| | | | | (SEQ | N52A; | | | | |
| | | | | ID | A55P; | | | | |
| | | | | NO: | T59M; | | | | |
| | | | | 6) | R61G; | | | | |
| | | | | | F64Y | | | | |
| 38 | | | HSA | GGSS | hCFI; | | | | |
| | | | | GG | N52A | | | | |
| | | | | (SEQ | | | | | |
| | | | | ID | | | | | |
| | | | | NO: | | | | | |
| | | | | 6) | | | | | |
| 39 | | | HSA | GGSS | hCFI; | | | | |
| | | | | GG | A55P | | | | |
| | | | | (SEQ | | | | | |
| | | | | ID | | | | | |
| | | | | NO: | | | | | |
| | | | | 6) | | | | | |
| 40 | | | HSA | GGSS | hCFI; | | | | |
| | | | | GG | T59E | | | | |
| | | | | (SEQ | | | | | |
| | | | | ID | | | | | |
| | | | | NO: | | | | | |
| | | | | 6) | | | | | |
| 41 | | | HSA | GGSS | hCFI; | | | | |
| | | | | GG | L73F | | | | |
| | | | | (SEQ | | | | | |
| | | | | ID | | | | | |
| | | | | NO: | | | | | |
| | | | | 6) | | | | | |
| 42 | | | HSA | GGSS | hCFI; | | | | |
| | | | | GG | L73D | | | | |
| | | | | (SEQ | | | | | |
| | | | | ID | | | | | |
| | | | | NO: | | | | | |
| | | | | 6) | | | | | |
| 43 | | | HSA | GGSS | hCFI; | | | | |
| | | | | GG | Δ(275- | | | | |
| | | | | (SEQ | CAGFASV | | | | |
| | | | | ID | AQE-284) | | | | |
| | | | | NO: | (SEQ ID | | | | |
| | | | | 6) | NO: 31) > | | | | |
| | | | | | CEEDRQQ | | | | |
| | | | | | NIPKGLA | | | | |
| | | | | | RSAQGEA | | | | |
| | | | | | EIETE | | | | |
| | | | | | (SEQ ID | | | | |
| | | | | | NO: 32) | | | | |
| 44 | | | HSA | GGSS | hCFI; | GGSS | hCR1; | | |
| | | | | GG | Δ(275- | GG | CCP15; | | |
| | | | | (SEQ | CAGFASV | (SEQ | CCP16; | | |
| | | | | ID | AQE-284) | ID NO: | CCP17 | | |
| | | | | NO: | (SEQ ID | 6) | | | |
| | | | | 6) | NO: 31) > | | | | |
| | | | | | CEEDRQQ | | | | |
| | | | | | NIPKGLA | | | | |
| | | | | | RSAQGEA | | | | |
| | | | | | EIETE | | | | |
| | | | | | (SEQ ID | | | | |
| | | | | | NO: 32) + | | | | |
| | | | | | GGSSGG | | | | |
| | | | | | (SEQ ID | | | | |
| | | | | | NO: 6) + | | | | |
| | | | | | CR1(ccp15- | | | | |
| | | | | | 17) | | | | |
| 45 | | | HSA | GGSS | hCFI; | | | | |
| | | | | GG | R484A | | | | |
| | | | | (SEQ | | | | | |
| | | | | ID | | | | | |
| | | | | NO: | | | | | |
| | | | | 6) | | | | | |
| 46 | | | HSA | GGSS | hCFI; | | | | |
| | | | | GG | R484K | | | | |
| | | | | (SEQ | | | | | |
| | | | | ID | | | | | |
| | | | | NO: | | | | | |
| | | | | 6) | | | | | |
| 47 | | | HSA | GGSS | hCFI; | | | | |
| | | | | GG | R484E | | | | |
| | | | | (SEQ | | | | | |
| | | | | ID | | | | | |
| | | | | NO: | | | | | |
| | | | | 6) | | | | | |
| 48 | | | HSA | GGSS | hCFI; | | | | |
| | | | | GG | Δ(V565) > | | | | |
| | | | | (SEQ | VG | | | | |
| | | | | ID | | | | | |
| | | | | NO: | | | | | |
| | | | | 6) | | | | | |
| 49 | | | HSA | GGSS | hCFI; | | | | |
| | | | | GG | Δ(V565) > | | | | |
| | | | | (SEQ | VY | | | | |
| | | | | ID | | | | | |
| | | | | NO: | | | | | |
| | | | | 6) | | | | | |
| 50 | | | HSA | GGSS | hCFI; | | | | |
| | | | | GG | E416K | | | | |
| | | | | (SEQ | | | | | |
| | | | | ID | | | | | |
| | | | | NO: | | | | | |
| | | | | 6) | | | | | |
| 51 | | | HSA | GGSS | hCFI; | | | | |
| | | | | GG | E416R | | | | |
| | | | | (SEQ | | | | | |
| | | | | ID | | | | | |
| | | | | NO: | | | | | |
| | | | | 6) | | | | | |
| 52 | | | HSA | GGSS | hCFI; | | | | |
| | | | | GG | D395A | | | | |
| | | | | (SEQ | | | | | |
| | | | | ID | | | | | |
| | | | | NO: | | | | | |
| | | | | 6) | | | | | |
| 53 | | | HSA | GGSS | hCFI; | | | | |
| | | | | GG | D395G | | | | |
| | | | | (SEQ | | | | | |
| | | | | ID | | | | | |
| | | | | NO: | | | | | |
| | | | | 6) | | | | | |
| 54 | | | HSA | GGSS | hCFI; | | | | |
| | | | | GG | D395K | | | | |
| | | | | (SEQ | | | | | |
| | | | | ID | | | | | |
| | | | | NO: | | | | | |
| | | | | 6) | | | | | |
| 55 | | | HSA | GGSS | hCFI; | | | | |
| | | | | GG | D395R | | | | |
| | | | | (SEQ | | | | | |
| | | | | ID | | | | | |
| | | | | NO: | | | | | |
| | | | | 6) | | | | | |
| 56 | | | HSA | GGSS | hCFI; | | | | |
| | | | | GG | D395A; | | | | |
| | | | | (SEQ | D425A | | | | |
| | | | | ID | | | | | |
| | | | | NO: | | | | | |
| | | | | 6) | | | | | |
| 57 | | | HSA | GGSS | hCFI; | | | | |
| | | | | GG | D395A; | | | | |
| | | | | (SEQ | E416A | | | | |
| | | | | ID | | | | | |
| | | | | NO: | | | | | |
| | | | | 6) | | | | | |
| 58 | | | HSA | GGSS | hCFI; | | | | |
| | | | | GG | D395A; | | | | |
| | | | | (SEQ | E416A; | | | | |
| | | | | ID | D425A | | | | |
| | | | | NO: | | | | | |
| | | | | 6) | | | | | |
| 59 | | | HSA | GGSS | hCFI; | | | | |
| | | | | GG | N402D | | | | |
| | | | | (SEQ | | | | | |
| | | | | ID | | | | | |
| | | | | NO: | | | | | |
| | | | | 6) | | | | | |
| 60 | | | HSA | GGSS | hCFI; | | | | |
| | | | | GG | K368D | | | | |
| | | | | (SEQ | | | | | |
| | | | | ID | | | | | |
| | | | | NO: | | | | | |
| | | | | 6) | | | | | |
| 63 | | | HSA | GGSS | hCFI | GGSS | hCR1; | GGSS | hFH; |
| | | | | GG | | GG | CCP15; | GGSS | CCP19; |
| | | | | (SEQ | | (SEQ | CCP16; | GGSS | CCP20 |
| | | | | ID | | ID NO: | CCP17 | GGSS | |
| | | | | NO: | | 6) | | GG | |
| | | | | 6) | | | | (SEQ | |
| | | | | | | | | ID NO: | |
| | | | | | | | | 33) | |
| 64 | | | HSA | GGSS | hCFI | GGSS | hCR1; | | |
| | | | | GG | | GG | CCP8; | | |
| | | | | (SEQ | | (SEQ | CCP9; | | |
| | | | | ID | | ID NO: | CCP10 | | |
| | | | | NO: | | 6) | | | |
| | | | | 6) | | | | | |
| 65 | | | HSA | GGSS | hCFI | GGSS | hCR1; | GGSS | hFH; |
| | | | | GG | | GG | CCP8; | GGSS | CCP19; |
| | | | | (SEQ | | (SEQ | CCP9; | GGSS | CCP20 |
| | | | | ID | | ID NO: | CCP10; | GGSS | |
| | | | | NO: | | 6) | hFH; | GG | |
| | | | | 6) | | | CCP4 | (SEQ | |
| | | | | | | | | ID NO: | |
| | | | | | | | | 33) | |
| 66 | | | HSA | GGSS | hCFI | GGSS | hCR1; | GGSS | hFH; |
| | | | | GG | | GG | CCP8; | GGSS | CCP19; |
| | | | | (SEQ | | (SEQ | CCP9; | GGSS | CCP20 |
| | | | | ID | | ID NO: | CCP10 | GGSS | |
| | | | | NO: | | 6) | | GG | |
| | | | | 6) | | | | (SEQ | |
| | | | | | | | | ID NO: | |
| | | | | | | | | 33) | |
| 67 | | | HSA | GGSS | hCFI | GGSS | hCR1; | | |
| | | | | GG | | GG | CCP1; | | |
| | | | | (SEQ | | (SEQ | CCP2; | | |
| | | | | ID | | ID NO: | CCP3; | | |
| | | | | NO: | | 6) | CCP4 | | |
| | | | | 6) | | | | | |
| 68 | | | HSA | GGSS | hCFI | GGSS | hCR1; | GGSS | hCR1; |
| | | | | GG | | GG | CCP1; | GG | CCP15; |
| | | | | (SEQ | | (SEQ | CCP2; | (SEQ | CCP16; |
| | | | | ID | | ID NO: | CCP3 | ID NO: | CCP17 |
| | | | | NO: | | 6) | | 6) | |
| | | | | 6) | | | | | |
| 70 | | | HSA | GGSS | Rat CFI | | | | |
| | | | | GGSS | | | | | |
| | | | | GG | | | | | |
| | | | | (SEQ | | | | | |
| | | | | ID | | | | | |
| | | | | NO: | | | | | |
| | | | | 34) | | | | | |
| 71 | | | HSA | GGSS | Gorilla | | | | |
| | | | | GGSS | CFI | | | | |
| | | | | GG | | | | | |
| | | | | (SEQ | | | | | |
| | | | | ID | | | | | |
| | | | | NO: | | | | | |
| | | | | 34) | | | | | |
| 72 | | | HSA | GGSS | Cyno CFI | | | | |
| | | | | GGSS | | | | | |
| | | | | GG | | | | | |
| | | | | (SEQ | | | | | |
| | | | | ID | | | | | |
| | | | | NO: | | | | | |
| | | | | 34) | | | | | |
| 73 | | | HSA | GGSS | Chimp CFI | | | | |
| | | | | GGSS | | | | | |
| | | | | GG | | | | | |
| | | | | (SEQ | | | | | |
| | | | | ID | | | | | |
| | | | | NO: | | | | | |
| | | | | 34) | | | | | |
| 74 | | | HSA | GGSS | Bovine CFI | | | | |
| | | | | GGSS | | | | | |
| | | | | GG | | | | | |
| | | | | (SEQ | | | | | |
| | | | | ID | | | | | |
| | | | | NO: | | | | | |
| | | | | 34) | | | | | |
| 75 | | | HSA | GGSS | Alligator | | | | |
| | | | | GGSS | CFI | | | | |
| | | | | GG | | | | | |
| | | | | (SEQ | | | | | |
| | | | | ID | | | | | |
| | | | | NO: | | | | | |
| | | | | 34) | | | | | |
| 76 | | | HSA | GGSS | hCFI; | | | | |
| | | | | GG | H383K; | | | | |
| | | | | (SEQ | D385N; | | | | |
| | | | | ID | K387S; | | | | |
| | | | | NO: | R388Q; | | | | |
| | | | | 6) | E392Q; | | | | |
| | | | | | Y393T; | | | | |
| | | | | | D395K; | | | | |
| | | | | | K419M; | | | | |
| | | | | | D420H; | | | | |
| | | | | | G421T; | | | | |
| | | | | | N422G; | | | | |
| | | | | | D425E | | | | |
| 77 | | | HSA | GGSS | hCFI; | | | | |
| | | | | GG | H383K; | | | | |
| | | | | (SEQ | R388Q; | | | | |
| | | | | ID | E392Q; | | | | |
| | | | | NO: | Y393T; | | | | |
| | | | | 6) | K419M | | | | |
| 78 | | | HSA | GGSS | hCFI; | | | | |
| | | | | GG | K51D; | | | | |
| | | | | (SEQ | T59E; | | | | |
| | | | | ID | R61D | | | | |
| | | | | NO: | | | | | |
| | | | | 6) | | | | | |
| 79 | | | HSA | GGSS | hCFI; | | | | |
| | | | | GG | Q69H; | | | | |
| | | | | (SEQ | L73F; | | | | |
| | | | | ID | G79E; | | | | |
| | | | | NO: | T80I; | | | | |
| | | | | 6) | L83S; | | | | |
| | | | | | N84H | | | | |
| 80 | | | HSA | GGSS | hCFI; Δ(1- | | | | |
| | | | | GG | KVTYTS- | | | | |
| | | | | (SEQ | 6)(SEQ ID | | | | |
| | | | | ID | NO: 35) > | | | | |
| | | | | NO: | RSPSASD | | | | |
| | | | | 6) | LP (SEQ | | | | |
| | | | | | ID NO: 36) | | | | |
| 81 | | | HSA | GGSS | hCFI; | | | | |
| | | | | GG | M186E; | | | | |
| | | | | (SEQ | G187L; | | | | |
| | | | | ID | Y188S; | | | | |
| | | | | NO: | Q189N; | | | | |
| | | | | 6) | D190G; | | | | |
| | | | | | F191L; | | | | |
| | | | | | D193G | | | | |
| 89 | | | HSA | GGSS | hCFI; | GGSS | hCR1; | GGGG | hFH; |
| | | | | GG | T377G; | GG | CCP15; | GGGG | CCP19; |
| | | | | (SEQ | E457G; | (SEQ | CCP16; | GGGG | CCP20 |
| | | | | ID | N531G | ID NO: | CCP17; | (SEQ | |
| | | | | NO: | | 6) | hFH; | ID NO: | |
| | | | | 6) | | | CCP4 | 37) | |
| 90 | HHH | GGSS | HSA | GGSS | hCFI; | | | | |
| | HHH | GGSS | | GG | T377G; | | | | |
| | SG | GGSS | | (SEQ | E457G; | | | | |
| | (SEQ | GGSS | | ID | N531G | | | | |
| | ID | GGSS | | NO: | | | | | |
| | NO: | GGSS | | 6) | | | | | |
| | 38); | GGSS | | | | | | | |
| | hCR1; | GGSS | | | | | | | |
| | CCP15; | GGSS | | | | | | | |
| | CCP16; | GGSS | | | | | | | |
| | CCP17; | GGSS | | | | | | | |
| | hFH; | GGSS | | | | | | | |
| | CCP4; | GGSS | | | | | | | |
| | GGG | GGSS | | | | | | | |
| | GGG | GG | | | | | | | |
| | GGG | (SEQ | | | | | | | |
| | GGG | ID | | | | | | | |
| | (SEQ | NO: | | | | | | | |
| | ID | 39) | | | | | | | |
| | NO: | | | | | | | | |
| | 37); | | | | | | | | |
| | hFH; | | | | | | | | |
| | CCP19; | | | | | | | | |
| | CCP20 | | | | | | | | |
| 91 | | | HSA | GGSS | hCFI; | | | | |
| | | | | GG | T377A; | | | | |
| | | | | (SEQ | E457G; | | | | |
| | | | | ID | N531G | | | | |
| | | | | NO: | | | | | |
| | | | | 6) | | | | | |
| 92 | | | HSA | GGSS | hCFI; | | | | |
| | | | | GG | N422A | | | | |
| | | | | (SEQ | | | | | |
| | | | | ID | | | | | |
| | | | | NO: | | | | | |
| | | | | 6) | | | | | |
| 93 | | | HSA | GGSS | hCFI; | | | | |
| | | | | GG | T377A | | | | |
| | | | | (SEQ | | | | | |
| | | | | ID | | | | | |
| | | | | NO: | | | | | |
| | | | | 6) | | | | | |
| 94 | | | | | hCFI-PD; | | | | |
| 95 | | | HSA | GGSS | hCFI; | | | | |
| | | | | GG | | | | | |
| | | | | (SEQ | | | | | |
| | | | | ID | | | | | |
| | | | | NO: | | | | | |
| | | | | 6) | | | | | |
| 96 | | | | | hCFI; | | | | |

In some embodiments, the first component comprises a wild type CFI of any species, either a full length or domain thereof (the “core” domain). In some embodiments, the first component comprises a CFI variant of the disclosure, described in detail in the preceding section, for example in Table 3, also provided in Table 2. It is noted that the second and subsequent components may increase the activity or alter the specificity of the CFI portion (first component) or its half-life. The second and subsequent components may also allow for CFI portion (first component) to act within the complement system without the presence of an exogenous cofactor (e.g. a cofactor such as Factor H (FH) or CR1). As used herein, an exogenous cofactor for CFI is one that is not fused to CFI. It should be understood that a fusion construct may act within the complement system without the presence of FH and/or CR1, but the activity of the fusion construct may also be further increased with the presence of FH, and/or CR1, either as a part of the fusion construct or provided exogenously.

Provided herein are fusion constructs comprising a first component comprising any one of the CFI variants provided herein. It should be understood that the CFI variant may be any one of the CFI variants presented in Table 2 and Table 3.

In some embodiments, the second and subsequent components of the fusion construct is a protein. In some embodiments, the second and/or subsequent components is not a protein.

The components of the fusion constructs of the disclosure may be held together by optional linkers, as pictured in FIG. 7. They may be of any suitable length of at least one amino acid. A linker may be a flexible linker, and may be a peptide of about 1 to about 20 amino acid residues in length, wherein the amino acid residues may comprise glycine residues. The linker may also optionally comprise serine residues. Exemplary flexible linkers can include, but are not limited to, glycine polymers, glycine-serine polymers, glycine-alanine polymers, alanine-serine polymers, or any other suitable flexible linkers known in the art. An exemplary linker is GGSSGGⁿ (SEQ ID NO: 51), wherein n is any number from about 1 to about 20. Other exemplary linkers include (GGSS)ⁿ, which denotes a linker of GGSS repeated n number of times (SEQ ID NO: 26): and (GGSS)ⁿGG, which denotes a linker of GGSS repeated n number of times followed by GG (SEQ ID NO: 27). In some embodiments, the linkers are protease-sensitive cleavable linkers. Exemplary linkers linking the fusion constructs can be 1-50, 5-50, 10-50, 15-50, 20-50, 25-50, 1-20, 2-20, 3-20, 4-20, 5-20, 6-20, 7-20, 8-20, 9-20, 10-20, 3-15, 3-10, 3-9, 3-8, 3-7, 3-6, 3-5, 4-15, 4-10, 4-9, 4-8, 4-7, 4-6, 4-5, 5-15, 5-10, 5-9, 5-8, 5-7, 5-6, 6-15, 6-10, 6-9, 6-8, or 6-7 amino acids in length.

### CFI+Half-Life Extender Fusion Constructs

In some embodiments, the fusion construct comprises a wild type CFI or CFI variant (first component), and a second component, and wherein the second component is a half-life extender. Because naturally occurring CFI has a relatively short half-life, it may be advantageous in some embodiments to increase the half-life of CFI. As used herein, “CFI” is used to connotate either the wild type CFI, or variants thereof. By using a second component that is a half-life extender, the activity of CFI may increase, or it may improve another characteristic of the CFI as compared to a wild type CFI. For example, a wild type CFI or a CFI variant may have their half-life extended by fusing the CFI to a half-life extender.

Exemplary half-life extenders include, but are not limited to albumin, such as human serum albumin, PEG, a non-biodegradable polymer, a biodegradable polymer, and Fc. In some embodiments, the second component is a protein, and is a half-life extender, such as albumin or Fc. In some embodiments, the second component is not a protein, and is a half-life extender, such as PEG. In some embodiments, the half-life extender is comprising peptide repeats.

In some embodiments, a second component is a half-life extender, and is albumin. It is noted that as used herein, albumin refers to any albumin such as any serum albumin, or an albumin variant, or albumin derivative. As an example, a variant of albumin includes any albumin comprising at least one modification corresponding to the amino acid sequence set forth in SEQ ID NO: 7 (wild type Human serum albumin (HSA)), or at least one modification corresponding to the amino acid sequence of an albumin of any non-human species. In exemplary embodiments, the albumin is human serum albumin (HSA) and is provided in SEQ ID NO: 7.

Exemplary fusion constructs comprising wild type CFI and HSA are referred to herein, as “CFI-HSA” and are discussed in further detail below.

In some embodiments, a fusion construct of the disclosure comprises albumin and a CFI variant of the disclosure. Exemplary constructs are provided in Table 3.

### Structural Arrangements of Fusion Constructs

Exemplary structural arrangements are depicted in FIG. 7.

In some embodiments, a wild type CFI or a CFI variant of the disclosure is the first component of a fusion construct, and wherein this CFI portion comprises an A chain and a B chain. In some embodiments, the fusion construct comprises a structural arrangement from N-terminus to C-terminus (A chain)-(optional linker)-(B chain)-(optional linker)-(Second Component). In some embodiments, the fusion construct comprises an inversion of the A and B chains in its CFI component, such that the structural arrangement from N-terminus to C-terminus, is (B chain)-(optional linker)-(A chain)-(optional linker)-(Second Component).

In some embodiments, a wild type CFI or a CFI variant of the disclosure is the first component of a fusion construct, and wherein this CFI portion comprises an A chain and a B chain. In some embodiments, the fusion construct comprises a structural arrangement from N-terminus to C-terminus, as (Second Component)-(optional linker)-(A chain)-(optional linker)-(B chain). In some embodiments, the fusion construct comprises an inversion of the A and B chains in its CFI component, such that the structural arrangement from N-terminus to C-terminus is (Second Component)-(optional linker)-(B chain)-(optional linker)-(A chain).

In some embodiments, provided herein are fusion constructs comprising at least a first component, wherein the first component is any of the wild type CFI or CFI variants provided herein (CFI portion), and a second component, wherein the first component and second component are fused, and wherein the second component is fused to the N-terminal end of the CFI portion. In some embodiments, the second component is fused to the C-terminal end of the CFI portion. In some embodiments, the second component is fused to the C-terminal end of the CFI portion, and a third component is further fused to the N-terminal end of the CFI portion. In some embodiments, the second component is fused to the N-terminal end of the CFI portion, and a third component is further fused to the C-terminal end of the CFI portion.

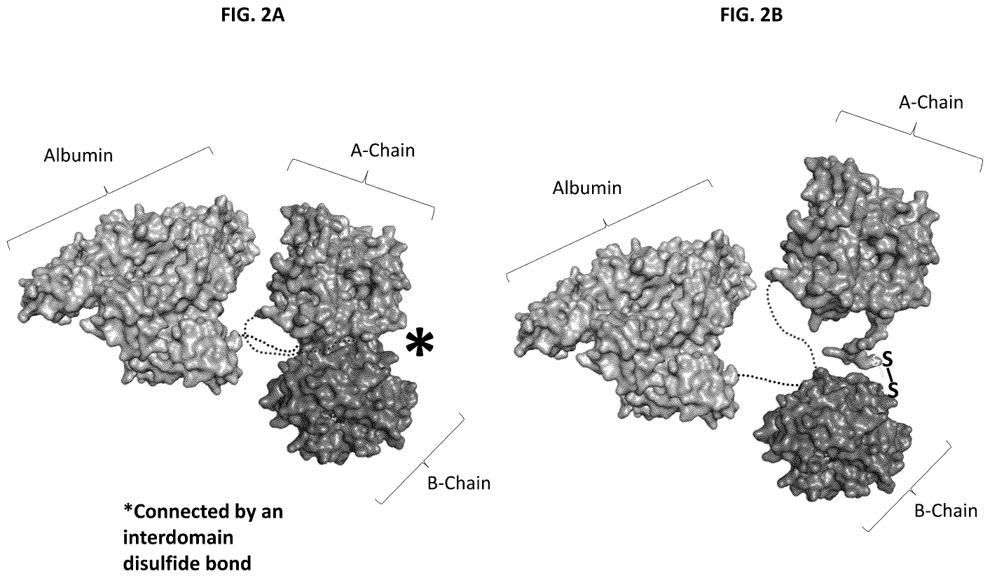

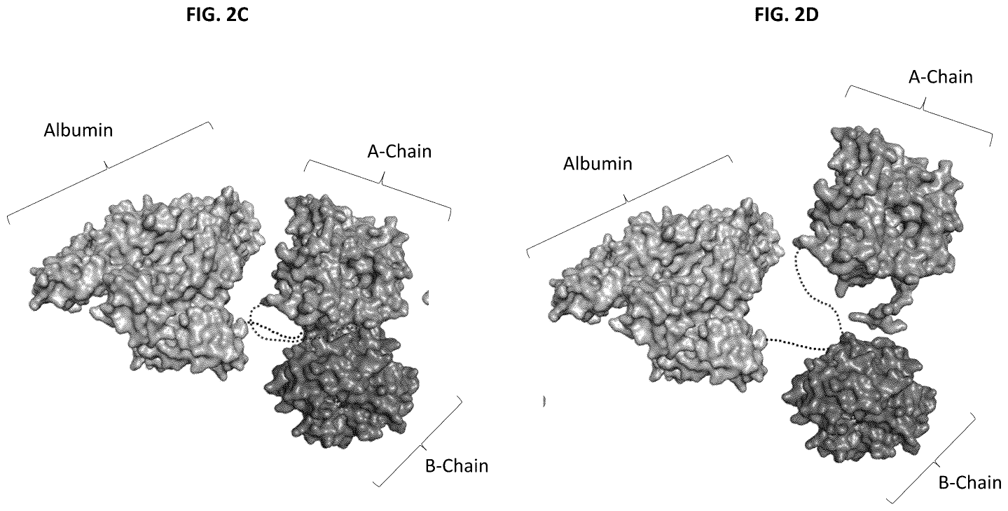

FIGS. 2A-2D depict models of a fusion construct comprising an albumin and a CFI variant, wherein the CFI variant comprises an A-B chain inversion. FIGS. 2A-2B depict a first version and FIGS. 2C-2D depict a second version of models of a fusion construct comprising human serum albumin (HSA) and the A and B chains of CFI, wherein the A and B chains comprise an inversion. The first version of an A-B chain inversion CFI variant comprises an inter-domain disulfide bond. The second version does not comprise the inter-domain disulfide bond. Both versions of the inversion variants may be constructed in a head-to-tail fashion as: (HSA)-(optional linker)-(B chain)-(optional linker)-(A chain).

Accordingly, provided herein are CFI variants, wherein the CFI variant is a first component of a fusion construct comprising a first component and a second component, and the CFI variant is fused to the second component, and wherein the CFI comprises an A chain and a B chain, and wherein the structural arrangement from N-terminus to C-terminus, or C-terminus to N-terminus, is (Second Component)-(optional linker)-(B chain)-(optional linker)-(A chain).

In some embodiments, the second component is at least one domain of Factor H. Fusion constructs comprising at least one CFI domain and Factor H are discussed in further detail below. In some embodiments, the second component is at least one domain of CR1. Fusion constructs comprising at least one CFI domain and Factor H are discuss in further detail below. In some embodiments, the second component comprises at least one domain of Factor H and at least one domain of CR1. Fusion constructs comprising at least one CFI domain, at least one Factor H domain, and at least one CR1 domain are discussed in further detail below.

### Components of Fusion Constructs

Provided herein are fusion constructs comprising a first component and additional components. In some embodiments, the first component comprises a wild type CFI, or a CFI variant of the disclosure. In some embodiments, the component comprises a half-life extender. In some embodiments, the second component comprises at least one domain of Factor H (FH), at least one domain of CR1, or a mixture of FH and CR1 domains. In some embodiments, the fusion construct further comprises additional components. In some embodiments, the first, second, and third (or more) components are any one or more of the components presented in Table 4. Table 4 presents various exemplary components and the amino acid sequences of the components that may be used to generate CFI fusion constructs provided herein.

Turning to Table 4, SEQ ID NO: 1 is the amino acid sequence of wild type plasma-derived human CFI, referred to as “CFI-PD”, and has a leader sequence. Wild type CFI used for fusion with a second component may comprise the amino acid sequence of SEQ ID NO: 5, which does not include the leader sequence present in SEQ ID NO: 1. A mouse Ig kappa chain V-III region MOPC 63 leader sequence (SEQ ID NO: 2) may instead be used for the recombinant production of any of the CFI fusion constructs provided herein. In some embodiments, provided herein are CFI fusion constructs comprising at least one CFI domain, wherein the at least one CFI domain comprises the amino acid sequence set forth in SEQ ID NO: 5.

TABLE 4
Components of Exemplary CFI Fusion Constructs

| Description | Sequence |
|---|---|
| Wild type | MKLLHVFLLFLCFHLRFCKV |
| plasma-derived | TYTSQEDLVEKKCLAKKYTH |
| human CFI (CFI- | LSCDKVFCQPWQRCIEGTCV |
| PD) | CKLPYQCPKNGTAVCATNRR |
| | SFPTYCQQKSLECLHPGTKF |
| | LNNGTCTAEGKFSVSLKHGN |
| | TDSEGIVEVKLVDQDKTMFI |
| | CKSSWSMREANVACLDLGFQ |
| | QGADTQRRFKLSDLSINSTE |
| | CLHVHCRGLETSLAECTFTK |
| | RRTMGYQDFADVVCYTQKAD |
| | SPMDDFFQCVNGKYISQMKA |
| | CDGINDCGDQSDELCCKACQ |
| | GKGFHCKSGVCIPSQYQCNG |
| | EVDCITGEDEVGCAGFASVT |
| | QEETEILTADMDAERRRIKS |
| | LLPKLSCGVKNRMHIRRKRI |
| | VGGKRAQLGDLPWQVAIKDA |
| | SGITCGGIYIGGCWILTAAH |
| | CLRASKTHRYQIWTTVVDWI |
| | HPDLKRIVIEYVDRIIFHEN |
| | YNAGTYQNDIALIEMKKDGN |
| | KKDCELPRSIPACVPWSPYL |
| | FQPNDTCIVSGWGREKDNER |
| | VFSLQWGEVKLISNCSKFYG |
| | NRFYEKEMECAGTYDGSIDA |
| | CKGDSGGPLVCMDANNVTYV |
| | WGVVSWGENCGKPEFPGVYT |
| | KVANYFDWISYHVGRPFISQ |
| | YNV |
| | (SEQ ID NO: 1) |
| Leader sequence | METDTLLLWVLLLWVPGSTG |
| (mouse leader for | (SEQ ID NO: 2) |
| CFI-HSA) | |
| Human CFI | MKLLHVFLLFLCFHLRFC |
| leader sequence | (SEQ ID NO: 3) |
| Human Factor H | MRLLAKIICLMLWAICVAED |
| (FH) | CNELPPRRNTEILTGSWSDQ |
| | TYPEGTQAIYKCRPGYRSLG |
| | NVIMVCRKGEWVALNPLRKC |
| | QKRPCGHPGDTPFGTFTLTG |
| | GNVFEYGVKAVYTCNEGYQL |
| | LGEINYRECDTDGWTNDIPI |
| | CEVVKCLPVTAPENGKIVSS |
| | AMEPDREYHFGQAVRFVCNS |
| | GYKIEGDEEMHCSDDGFWSK |
| | EKPKCVEISCKSPDVINGSP |
| | ISQKIIYKENERFQYKCNMG |
| | YEYSERGDAVCTESGWRPLP |
| | SCEEKSCDNPYIPNGDYSPL |
| | RIKHRTGDEITYQCRNGFYP |
| | ATRGNTAKCTSTGWIPAPRC |
| | TLKPCDYPDIKHGGLYHENM |
| | RRPYFPVAVGKYYSYYCDEH |
| | FETPSGSYWDHIHCTQDGWS |
| | PAVPCLRKCYFPYLENGYNQ |
| | NYGRKFVQGKSIDVACHPGY |
| | ALPKAQTTVTCMENGWSPTP |
| | RCIRVKTCSKSSIDIENGFI |
| | SESQYTYALKEKAKYQCKLG |
| | YVTADGETSGSITCGKDGWS |
| | AQPTCIKSCDIPVFMNARTK |
| | NDFTWFKLNDTLDYECHDGY |
| | ESNTGSTTGSIVCGYNGWSD |
| | LPICYERECELPKIDVHLVP |
| | DRKKDQYKVGEVLKFSCKPG |
| | FTIVGPNSVQCYHFGLSPDL |
| | PICKEQVQSCGPPPELLNGN |
| | VKEKTKEEYGHSEVVEYYCN |
| | PRFLMKGPNKIQCVDGEWTT |
| | LPVCIVEESTCGDIPELEHG |
| | WAQLSSPPYYYGDSVEFNCS |
| | ESFTMIGHRSITCIHGVWTQ |
| | LPQCVAIDKLKKCKSSNLII |
| | LEEHLKNKKEFDHNSNIRYR |
| | CRGKEGWIHTVCINGRWDPE |
| | VNCSMAQIQLCPPPPQIPNS |
| | HNMTTTLNYRDGEKVSVLCQ |
| | ENYLIQEGEEITCKDGRWQS |
| | IPLCVEKIPCSQPPQIEHGT |
| | INSSRSSQESYAHGTKLSYT |
| | CEGGFRISEEN |
| | ETTCYMGKWSSPPQCEGLPC |
| | KSPPEISHGVVAHMSDSYQY |
| | GEEVTYKCFEGFGIDGPAIA |
| | KCLGEKWSHPPSCIKTDCLS |
| | LPSFENAIPMGEKKDVYKAG |
| | EQVTYTCATYYKMDGASNVT |
| | CINSRWTGRPTCRDTSCVNP |
| | PTVQNAYIVSRQMSKYPSGE |
| | RVRYQCRSPYEMFGDEEVMC |
| | LNGNWTEPPQCKDSTGKCGP |
| | PPPIDNGDITSFPLSVYAPA |
| | SSVEYQCQNLYQLEGNKRIT |
| | CRNGQWSEPPKCLHPCVISR |
| | EIMENYNIALRWTAKQKLYS |
| | RTGESVEFVCKRGYRLSSRS |
| | HTLRTTCWDGKLEYPTCAKR |
| | (SEQ ID NO: 4) |
| Human mini | MRLLAKIICLMLWAICVAED |
| Factor H (mini | CNELPPRRNTEILTGSWSDQ |
| FH) | TYPEGTQAIYKCRPGYRSLG |
| | NIIMVCRKGEWVALNPLRKC |
| | QKRPCGHPGDTPFGTFTLTG |
| | GNVFEYGVKAVYTCNEGYQL |
| | LGEINYRECDTDGWTNDIPI |
| | CEVVKCLPVTAPENGKIVSS |
| | AMEPDREYHFGQAVRFVCNS |
| | GYKIEGDEEMHCSDDGFWSK |
| | EKPKCVEISCKSPDVINGSP |
| | ISQKIIYKENERFQYKCNMG |
| | YEYSERGDAVCTESGWRPLP |
| | SCEEAGGGGGGGGGGGGGKC |
| | GPPPPIDNGDITSFPLSVYA |
| | PASSVEYQCONLYQLEGNKR |
| | ITCRNGQWSEPPKCLHPCVI |
| | SREIMENYNIALRWTAKQKL |
| | YSRTGESVEFVCKRGYRLSS |
| | RSHTLRTTCWDGKLEYPTCA |
| | KRENLYFQGHHHHHH |
| | (SEQ ID NO: 8) |
| Wild type CFI of | KVTYTSQEDLVEKKCLAKKY |
| SEQ ID NO 1 | THLSCDKVFCQPWQRCIEGT |
| without signal | CVCKLPYQCPKNGTAVCATN |
| sequence | RRSFPTYCQQKSLECLHPGT |
| | KFLNNGTCTAEGKFSVSLKH |
| | GNTDSEGIVEVKLVDQDKTM |
| | FICKSSWSMREANVACLDLG |
| | FQQGADTQRRFKLSDLSINS |
| | TECLHVHCRGLETSLAECTF |
| | TKRRTMGYQDFADVVCYTQK |
| | ADSPMDDFFQCVNGKYISQM |
| | KACDGINDCGDQSDELCCKA |
| | CQGKGFHCKSGVCIPSQYQC |
| | NGEVDCITGEDEVGCAGFAS |
| | VTQEETEILTADMDAERRRI |
| | KSLLPKLSCGVKNRMHIRRK |
| | RIVGGKRAQLGDLPWQVAIK |
| | DASGITCGGIYIGGCWILTA |
| | AHCLRASKTHRYQIWTTVVD |
| | WIHPDLKRIVIEYVDRIIFH |
| | ENYNAGTYQNDIALIEMKKD |
| | GNKKDCELPRSIPACVPWSP |
| | YLFQPNDTCIVSGWGREKDN |
| | ERVFSLQWGEVKLISNCSKF |
| | YGNRFYEKEMECAGTYDGSI |
| | DACKGDSGGPLVCMDANNVT |
| | YVWGVVSWGENCGKPEFPGV |
| | YTKVANYFDWISYHVGRPFI |
| | SQYNV |
| | (SEQ ID NO: 5) |
| Linker | GGSSGG (SEQ ID NO: 6) |
| Human serum | DAHKSEVAHRFKDLGEENFK |
| albumin (HSA) | ALVLIAFAQYLQQCPFEDHV |
| | KLVNEVTEFAKTCVADESAE |
| | NCDKSLHTLFGDKLCTVATL |
| | RETYGEMADCCAKQEPERNE |
| | CFLQHKDDNPNLPRLVRPEV |
| | DVMCTAFHDNEETFLKKYLY |
| | EIARRHPYFYAPELLFFAKR |
| | YKAAFTECCQAADKAACLLP |
| | KLDELRDEGKASSAKQRLKC |
| | ASLQKFGERAFKAWAVARLS |
| | QRFPKAEFAEVSKLVTDLTK |
| | VHTECCHGDLLECADDRADL |
| | AKYICENQDSISSKLKECCE |
| | KPLLEKSHCIAEVENDEMPA |
| | DLPSLAADFVESKDVCKNYA |
| | EAKDVFLGMFLYEYARRHPD |
| | YSVVLLLRLAKTYETTLEKC |
| | CAAADPHECYAKVFDEFKPL |
| | VEEPQNLIKQNCELFEQLGE |
| | YKFQNALLVRYTKKVPQVST |
| | PTLVEVSRNLGKVGSKCCKH |
| | PEAKRMPCAEDYLSVVLNQL |
| | CVLHEKTPVSDRVTKCCTES |
| | LVNRRPCFSALEVDETYVPK |
| | EFNAETFTFHADICTLSEKE |
| | RQIKKQTALVELVKHKPKAT |
| | KEQLKAVMDDFAAFVEKCCK |
| | ADDKETCFAEEGKKLVAASQ |
| | AALGL |
| | (SEQ ID NO: 7) |
| HSA linked with | DAHKSEVAHRFKDLGEENFK |
| CFI (CFI-HSA) | ALVLIAFAQYLQQCPFEDHV |
| | KLVNEVTEFAKTCVADESAE |
| | NCDKSLHTLFGDKLCTVATL |
| | RETYGEMADCCAKQEPERNE |
| | CFLQHKDDNPNLPRLVRPEV |
| | DVMCTAFHDNEETFLKKYLY |
| | EIARRHPYFYAPELLFFAKR |
| | YKAAFTECCQAADKAACLLP |
| | KLDELRDEGKASSAKQRLKC |
| | ASLQKFGERAFKAWAVARLS |
| | QRFPKAEFAEVSKLVTDLTK |
| | VHTECCHGDLLECADDRADL |
| | AKYICENQDSISSKLKECCE |
| | KPLLEKSHCIAEVENDEMPA |
| | DLPSLAADFVESKDVCKNYA |
| | EAKDVFLGMFLYEYARRHPD |
| | YSVVLLLRLAKTYETTLEKC |
| | CAAADPHECYAKVFDEFKPL |
| | VEEPQNLIKQNCELFEQLGE |
| | YKFQNALLVRYTKKVPQVST |
| | PTLVEVSRNLGKVGSKCCKH |
| | PEAKRMPCAEDYLSVVLNQL |
| | CVLHEKTPVSDRVTKCCTES |
| | LVNRRPCFSALEVDETYVPK |
| | EFNAETFTFHADICTLSEKE |
| | RQIKKQTALVELVKHKPKAT |
| | KEQLKAVMDDFAAFVEKCCK |
| | ADDKETCFAEEGKKLVAASQ |
| | AALGLGGSSGGKVTYTSQED |
| | LVEKKCLAKKYTHLSCDKVF |
| | CQPWQRCIEGTCVCKLPYQC |
| | PKNGTAVCATNRRSFPTYCQ |
| | QKSLECLHPGTKFLNNGTCT |
| | AEGKFSVSLKHGNTDSEGIV |
| | EVKLVDQDKTMFICKSSWSM |
| | REANVACLDLGFQQGADTQR |
| | RFKLSDLSINSTECLHVHCR |
| | GLETSLAECTFTKRRTMGYQ |
| | DFADVVCYTQKADSPMDDFF |
| | QCVNGKYISQMKACDGINDC |
| | GDQSDELCCKACQGKGFHCK |
| | SGVCIPSQYQCNGEVDCITG |
| | EDEVGCAGFASVTQEETEIL |
| | TADMDAERRRIKSLLPKLSC |
| | GVKNRMHIRRKRIVGGKRAQ |
| | LGDLPWQVAIKDASGITCGG |
| | IYIGGCWILTAAHCLRASKT |
| | HRYQIWTTVVDWIHPDLKRI |
| | VIEYVDRIIFHENYNAGTYQ |
| | NDIALIEMKKDGNKKDCELP |
| | RSIPACVPWSPYLFQPNDTC |
| | IVSGWGREKDNERVFSLQWG |
| | EVKLISNCSKFYGNRFYEKE |
| | MECAGTYDGSIDACKGDSGG |
| | PLVCMDANNVTYVWGVVSWG |
| | ENCGKPEFPGVYTKVANYFD |
| | WISYHVGRPFISQYNV |
| | (SEQ ID NO: 21, Const. |
| | No. 95 from Table 3) |

### C. Complement Factor I and Albumin Fusion Constructs

### CFI Variants—Albumin Fusion Constructs

In some embodiments, provided herein are fusion constructs comprising a first component that is a CFI variant of the disclosure (e.g see Table 2), and second component that is albumin, e.g. serum albumin, e.g. human serum albumin.

In some embodiments, provided herein are fusion constructs comprising at least one CFI domain. and a second component, wherein the second component is HSA. and wherein the at least one CFI domain comprises any one or more domains of CFI selected from: the SPD, the FIMAC domain, the SRCR domain, the LDLr1, and the LDLr2 domains. In some embodiments, the any one or more domains of CFI comprise the amino acid sequence set forth in SEQ ID NO: 5. or comprise an amino acid sequence derived from SEQ ID NO: 5. In some embodiments, the any one or more domains of CFI correspond to the domains of a wild type CFI. In some embodiments, the at least one CFI domain comprises each one of the SPD, the FIMAC domain, the SRCR domain, and the LDLr1 and LDLr2 domains. In some embodiments, the at least one CFI domain of the CFI-HSA construct comprises only the SPD.

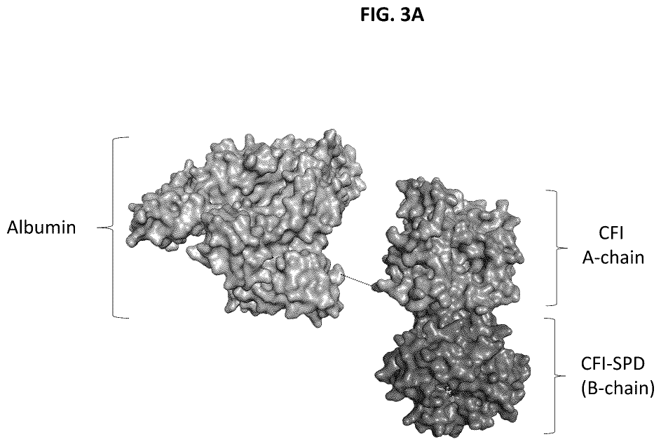

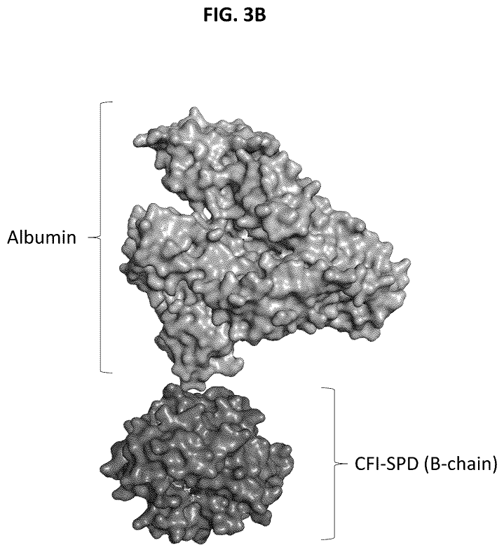

FIG. 3A depicts a model of an exemplary fusion construct comprising HSA fused with CFI, wherein the CFI comprises a CFI variant, comprising each one of the SPD, the FIMAC domain, the SRCR domain. and the LDLr1 and LDLr2 domains. Thus, the A-chain and B-chain are both included in the CFI in this model. The FIMAC domain, the SRCR domain. and the LDLr1 and LDLr2 domains together are the A-chain, or heavy chain, while the SPD is the B-chain, or light chain. In some embodiments, the amino acid residues of any one or more of domains of the fusion construct may correspond to that of a wild type CFI. In some embodiments, the amino acid residues of any one or more of domains of the fusion construct may comprise one or more modifications with respect to the domains of a wild type CFI.

FIG. 3B depicts a model of an exemplary fusion construct comprising HSA fused with a CFI portion, wherein the CFI comprises only the serine protease domain (SPD). The exemplary fusion construct depicted in FIG. 3B may be referred to as “HSA-SPD.” and includes an activation loop at amino acid residues 322-326. an autolysis loop at amino acid residues 455-463. and an S1 entrance frame at amino acid residues 529-536. In some embodiments, the amino acid residues of any one or more of the activation loop, the autolysis loop, and the S1 entrance frame of the fusion construct may correspond to that of a wild type SPD of CFI. In some embodiments, the amino acid residues of any one or more of the activation loop, the autolysis loop. and the S1 entrance frame of the fusion construct may comprise one or more modifications with respect to a wild type SPD of CFI.

### D. Complement Factor I and Factor H Fusion Constructs

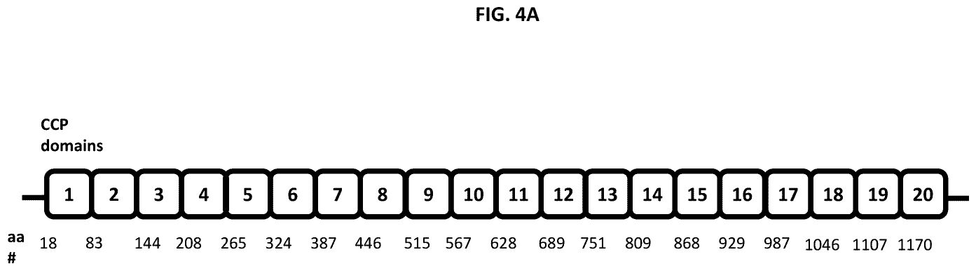

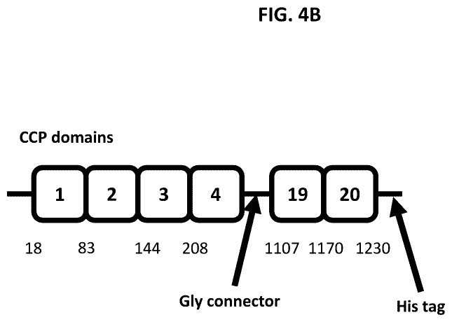

In some embodiments, provided herein are fusion constructs comprising a wild type CFI (or variant thereof) fused to at least one domain of Factor H. Factor H (FH), like CFI, is a protein involved in the complement pathway. FH is cofactor of CFI that forms a complex with CFI and C3b to catalyze C3b cleavage by CFI. As noted above, full-length FH comprises 20 domains. FIG. 4A depicts a schematic diagram of FH showing its 20 domains, each of which is a complement control protein (CCP) domain, and each of which are connected by short linkers, in a head-to-tail arrangement. The CCP domains are numbered 1-20 beginning at the N-terminus. CCPs 1-4 complex with C3b, and CCPs 19-20 complex with C3d. Without being bound to any theory or mechanism, FH is thought to be important for efficient C3b cleavage by CFI. Accordingly, in some embodiments, a fusion construct comprising specific domains of FH fused to at least one CFI domain may allow for C3b cleavage independent of exogenous FH. Exogenous FH may be defined as any FH that is not fused to any CFI domain, and may be a wild type FH. A wild type FH as used herein refers to any naturally occurring FH which is not a disease-causing FH. In some embodiments, the FH is a human FH. In some embodiments, the wild type FH comprises the amino acid sequence set forth in SEQ ID NO: 4.

In some embodiments, the additional component of the fusion constructs of the disclosure is at least one Factor H domain, or part of a domain of FH. In some embodiments, the at least one FH domain comprises CCP domains 1-20 of FH. In some embodiments, the at least one domain of FH correspond to that of a wild type FH comprising the amino acid sequence set forth in SEQ ID NO: 4.

In some embodiments, provided herein are fusion constructs comprising at least one CFI domain and an additional component, wherein the additional component is at least one Factor H domain, and wherein the at least one Factor H domain comprises complement control protein (CCP) domains 1-4 and 19-20 of Factor H. The CCP domains 1-4 and 19-20 are referred to as “mini Factor H” (mini FH). FIG. 4B depicts a schematic diagram of mini FH showing the CCP domains 1-4 connected by a Gly connector to domains 19-20, which include a His tag. In some embodiments, the mini FH is a human mini FH. In some embodiments, the amino acid sequence of mini FH comprises the amino acid sequence set forth in SEQ ID NO: 8.

Based on the structure of the complex formed by C3b-CFI and mini FH, several domains relevant for the function of FH are available. The following types of exemplary FH-CFI fusion constructs were generated as base molecules in order to drive FH-independent CFI cleavage activity:
(a) FH domains 1-8 fused with CFI (Factor H-CCPs1-8+CFI)
(b) FH domains 1-4 fused with CFI (Factor H-CCPs1-4+CFI)
(c) FH domains 19-20 fused with CFI (Factor H-CCPs19-20+CFI)
(d) FH domains 5-8 fused with CFI (Factor H-CCPs1-4+19-20+5-8+CFI)
(e) FH domains 1-4, 19-20, and/or 5-8 fused with CFI
(f) FH domains 1-4 and 19-20 fused with CFI (Factor H-CCPs1-4+19-20+CFI)
(g) FH domains 1-4, 19-20, and 5-8 fused with CFI (Factor H-CCPs1-4+19-20+5-8+CFI)
(h) FH domains 1-8 fused with only the LDLr2 CFI domain (Factor H-CCPs1-8+LDLR2-CFI)
(i) FH domains 1-4, 19-20, and 5-8 fused with only the LDLr2 CFI domain (Factor H-CCPs1-4+19-20+5-8+LDLr2-CFI)
(j) FH domains 1-4 fused with human serum albumin (HSA) and the serine protease domain (SPD) of CFI (CFI-HSA (SPD)-factor H-CCP1-4)
(k) FH domains 2-4 fused with human serum albumin (HSA) and the serine protease domain (SPD) of CFI (CFI-HSA (SPD)-factor H-CCP2-4)
(l) FH domains 2-3 fused with human serum albumin (HSA) and the serine protease domain (SPD) of CFI (CFI-HSA (SPD)-factor H-CCP2-3).

The above fusions can further be fused to albumin, e.g. HAS.

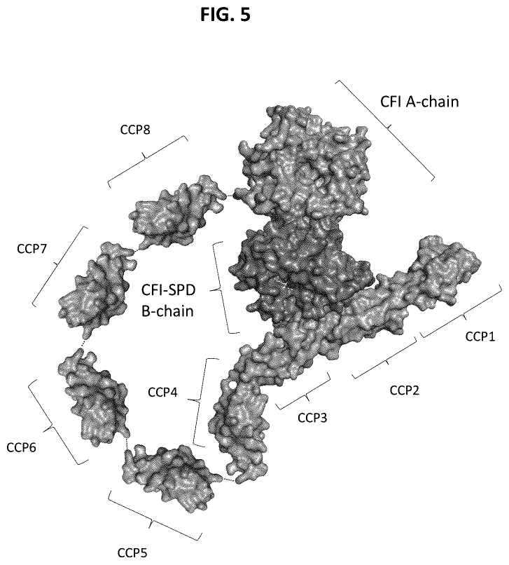

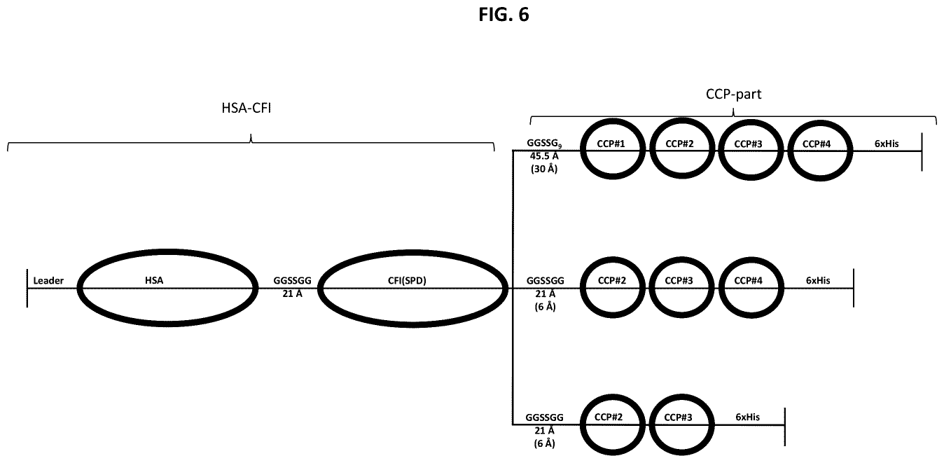

FIG. 5 depicts a model of an exemplary fusion construct comprising FH and CFI, comprising CCP domains 1-8 of FH fused with CFI, wherein the FH portion of the fusion construct is a truncated mini FH, and wherein the CFI comprises a wild type CFI. The wild type CFI comprises each one of the SPD, the FIMAC domain, the SRCR domain, the LDLr1, and the LDLr2 domain. The exemplary fusion construct shown in FIG. 5 is also referred to herein as Factor H-CPPs1-8+CFI. The A-chain and B-chain are both included in the CFI. The FIMAC domain, the SRCR domain, and the LDLr1 and LDLr2domains together are the A-chain, or heavy chain, while the SPD is the B-chain, or light chain. The FH comprises domains 1-4 and a linker comprising domains 5-8. In some embodiments, the amino acid residues of any one or more of domains of the FH and/or the CFI of the fusion construct may correspond to that of a wild type FH or a wild type CFI, respectively. In some embodiments, the amino acid residues of any one or more of domains of the FH and/or the CFI of the fusion construct may comprise one or more modifications with respect to the domains of a wild type FH or a wild type CFI, respectively.

Table 5A lists exemplary Factor H-containing fusion constructs.

TABLE 5A
Factor H Fusion-Containing Construct Base Molecules

| | Base Molecule- | |
|---|---|---|
| Alteration | Core Domain | Region/variant name |
| FH_CCP1-8 + | FH + wt hCFI | FH-CFI fusion (fusion #1) |
| GGGGGGGGGGGG (SEQ ID NO: | | |
| 37) + ΔHSA | | |
| FH_CCP1-4 + 19-20 + 5-8 + | | FH-CFI fusion (fusion #2) |
| GGGGGGGGGGGG (SEQ ID NO: | | |
| 37) + ΔHSA | | |
| Δ(K1-P305) + GGSSGG (SEQ ID | CFI-SPD + FH | HSA-SPD (ΔA-chain)-FH_CCP1-4 |
| NO: 6) + FH_CCP1-4 | | fusion |
| Δ(K1-P305) + GGSSGG (SEQ ID | | HSA-SPD (ΔA-chain)-FH_CCP2-4 |
| NO: 6) + FH_CCP2-4 | | fusion |
| Δ(K1-P305) + GGSSGG (SEQ ID | | HSA-SPD (ΔA-chain)-FH_CCP2-3 |
| NO: 6) + FH_CCP2-3 | | fusion |
| WT + GGSSGG (SEQ ID NO: 6) + | | CFI-HSA-FH_CCP1-4 fusion |
| CCP_1-4 | | |
| WT + GGSSGG (SEQ ID NO: 6) + | | CFI-HSA-FH_CCP2-3 fusion |
| CCP_2-3 | | |
| WT + GGSSGG (SEQ ID NO: 6) + | | CFI-HSA-FH_CCP2-4 fusion |
| CCP_2-4 | | |
| FH_CCP1-4 + G(43) (SEQ ID NO: | FH + wt hCFI | FH-CFI fusion (fusion #1) |
| 40) + wt hCFI | | (100%Gly, 150Å) |
| FH_CCP1-4 + GGGGSS(7) (SEQ | | FH-CFI fusion (fusion #1) |
| ID NO: 41) + wt hCFI | | (66%Gly, 150Å) |
| FH_CCP1-4 + GGSS(11) (SEQ ID | | FH-CFI fusion (fusion #1) |
| NO: 42) + wt hCFI | | (50%Gly, 150Å) |
| FH_CCP1-4 + G(53) (SEQ ID NO: | | FH-CFI fusion (fusion #1) |
| 43) + wt hCFI | | (100%Gly, 185Å) |
| FH_CCP1-4 + GGGGSS(9) (SEQ | | FH-CFI fusion (fusion #1) |
| ID NO: 44) + wt hCFI | | (66%Gly, 185Å) |
| FH_CCP1-4 + GGSS(13) (SEQ ID | | FH-CFI fusion (fusion #1) |
| NO: 45) + wt hCFI | | (50%Gly, 185Å) |
| FH_CCP2-4 + FH_CCP5-8 + | | FH-CFI fusion (fusion #1- |
| GGGGGGGGGGGG (SEQ ID NO: | | derivatives) |
| 37) + ΔHSA | | |
| FH_CCP1-3 + FH_CCP5-8 + | | FH-CFI fusion (fusion #1- |
| GGGGGGGGGGGG (SEQ ID NO: | | derivatives) |
| 37) + ΔHSA | | |
| FH_CCP2-3 + FH_CCP5-8 + | | FH-CFI fusion (fusion #1- |
| GGGGGGGGGGGG (SEQ ID NO: | | derivatives) |
| 37) + ΔHSA | | |
| WT + GGSSGG (SEQ ID NO: 6) + | | CFI-HSA-FH-CCP 1-4 with |
| CCP_1-4 + GGSS(6) + G (SEQ ID | | compstatin |
| NO: 46) + compstatin | | |
| WT + GGSSGG (SEQ ID NO: 6) + | | CFI-HSA-FH-CCP 1-5 with |
| CCP_1-5 + GGSS(3) + GGG (SEQ | | compstatin |
| ID NO: 47) + compstatin | | |
| Mouse mini Factor H | mouse FH | mFH |
| Human mini Factor H | human FH | mFH |

In some embodiments, a CFI variant is a first component of a fusion construct comprising a first component and a second component, and the CFI variant is fused to the second component, wherein the second component is at least one Factor H domain, wherein the FH domain comprises CCP 1-4 of FH.

In some embodiments, a CFI variant is a first component of a fusion construct comprising a first component and a second component, and the CFI variant is fused to the second component, wherein the second component is at least one Factor H domain, wherein the FH domain comprises CCP 1-4 of FH.

CR1 fusion constructs is discussed in the section below. It is noted that FH and CR1 chimeric constructs can further be generated and act as one component of the fusion constructs of the disclosure. For example, and referring to Construct No. 90 of Table 3, such a component may be: HHHHHHSG (SEQ ID NO: 38);hCR1;CCP15;CCP16;CCP17;hFH;CCP4;GGGGGGGGGGGG (SEQ ID NO: 37);hFH;CCP19;CCP20.

### E. Complement Factor I and Complement Receptor 1 Fusion Constructs

In some embodiments, provided herein are fusion constructs comprising a wild type CFI (or variant thereof) fused to at least one domain of Complement Receptor 1 (CR1). CR1 is also referred to as CD35. CR1, like CFI, is a protein involved in the complement pathway. CR1 is a cofactor of CFI. Accordingly, in some embodiments, a fusion construct comprising specific domains of CR1 fused to at least one CFI domain may allow for C3b and/or C4b cleavage independent of exogenous cofactor. An exogenous CR1 cofactor may be defined as any CR1 or portion thereof that is not fused to any CFI domain, and may be a wild type CR1, or may be CCP domains 1-3 or 15-17 of CR1. A wild type CR1 as used herein refers to any naturally occurring CR1 which is not a disease-causing CR1. In some embodiments, the CR1 is a human CR1.

In some embodiments, the at least second component of the fusion constructs of the disclosure is at least one CR1 domain, or part of a domain of CR1. In some embodiments, the at least one CR1 domain comprises CCP domains 8-10 of CR1. In some embodiment the at least one CR1 domain comprises CCP domains 15-17 of CR1. In some embodiments, the at least one CR1 domain comprises CCP domains 1-3of CR1. In some embodiments, the fusion constructs of the disclosure comprising at least one CR1domain also include fusion with albumin. In some embodiments, the fusion constructs of the disclosure comprising at least one CR1 domain also include fusion with albumin, and/or at least one domain of Factor H. In some embodiments, the at least one CR1 domain comprises CR1 CCP domain 15. In some embodiments, the at least one CR1 domain comprises CR1 CCP domain 16. In some embodiments, the at least one CR1 domain comprises CR1 CCP domain 17. In some embodiments, the at least one CR1domain comprises CR1 CCP domains 15-16. In some embodiments, the at least one CR1 domain comprises CR1 CCP domains 16-17.

Table 5B lists exemplary CR1-containing fusion constructs and the corresponding sequence of an exemplary fusion construct comprising a wild type CFI and CR1 CCP domains 15-17.

TABLE 5B
Complement Factor 1 Fusion-
Containing Constructs

| | | Fragments | |
|---|---|---|---|
| | | or Base | |
| Fusion | Terminal | Molecules | |
| Construct | fused | used | Sequence |
| HSA + CfI + | C | GGSSGG + | METDTLLLWVLL |
| GGSSGG + | | CR1 | LWVPGSTGDAHK |
| CR1(ccp15- | | (ccp15-17) | SEVAHRFKDLGE |
| 17) | | | ENFKALVLIAFA |
| | | | QYLQQCPFEDHVKL |
| | | | VNEVTEFAKTCVAD |
| | | | ESAENCDKSLHTLF |
| | | | GDKLCTVATLRETY |
| | | | GEMADCCAKQEPER |
| | | | NECFLQHKDDNPNL |
| | | | PRLVRPEVDVMCTA |
| | | | FHDNEETFLKKYLY |
| | | | EIARRHPYFYAPEL |
| | | | LFFAKRYKAAFTEC |
| | | | CQAADKAACLLPKL |
| | | | DELRDEGKASSAKQ |
| | | | RLKCASLQKFGERA |
| | | | FKAWAVARLSQRFP |
| | | | KAEFAEVSKLVTDL |
| | | | TKVHTECCHGDLLE |
| | | | CADDRADLAKYICE |
| | | | NQDSISSKLKECCE |
| | | | KPLLEKSHCIAEVE |
| | | | NDEMPADLPSLAAD |
| | | | FVESKDVCKNYAEA |
| | | | KDVFLGMFLYEYAR |
| | | | RHPDYSVVLLLRLA |
| | | | KTYETTLEKCCAAA |
| | | | DPHECYAKVFDEFK |
| | | | PLVEEPQNLIKQNC |
| | | | ELFEQLGEYKFQNA |
| | | | LLVRYTKKVPQVST |
| | | | PTLVEVSRNLGKVG |
| | | | SKCCKHPEAKRMPC |
| | | | AEDYLSVVLNQLCV |
| | | | LHEKTPVSDRVTKC |
| | | | CTESLVNRRPCFSA |
| | | | LEVDETYVPKEFNA |
| | | | ETFTFHADICTLSE |
| | | | KERQIKKQTALVEL |
| | | | VKHKPKATKEQLKA |
| | | | VMDDFAAFVEKCCK |
| | | | ADDKETCFAEEGKK |
| | | | LVAASQAALGLGGS |
| | | | SGGKVTYTSQEDLV |
| | | | EKKCLAKKYTHLSC |
| | | | DKVFCQPWQRCIEG |
| | | | TCVCKLPYQCPKNG |
| | | | TAVCATNRRSFPTY |
| | | | CQQKSLECLHPGTK |
| | | | FLNNGTCTAEGKFS |
| | | | VSLKHGNTDSEGIV |
| | | | EVKLVDQDKTMFIC |
| | | | KSSWSMREANVACL |
| | | | DLGFQQGADTQRRF |
| | | | KLSDLSINSTECLH |
| | | | VHCRGLETSLAECT |
| | | | FTKRRTMGYQDFAD |
| | | | VVCYTQKADSPMDD |
| | | | FFQCVNGKYISQMK |
| | | | ACDGINDCGDQSDE |
| | | | LCCKACQGKGFHCK |
| | | | SGVCIPSQYQCNGE |
| | | | VDCITGEDEVGCAG |
| | | | FASVTQEETEILTA |
| | | | DMDAERRRIKSLLP |
| | | | KLSCGVKNRMHIRR |
| | | | KRIVGGKRAQLGDL |
| | | | PWQVAIKDASGITC |
| | | | GGIYIGGCWILTAA |
| | | | HCLRASKTHRYQIW |
| | | | TTVVDWIHPDLKRI |
| | | | VIEYVDRIIFHENY |
| | | | NAGTYQNDIALIEM |
| | | | KKDGNKKDCELPRS |
| | | | IPACVPWSPYLFQP |
| | | | NDTCIVSGWGREKD |
| | | | NERVFSLQWGEVKL |
| | | | ISNCSKFYGNRFYE |
| | | | KEMECAGTYDGSID |
| | | | ACKGDSGGPLVCMD |
| | | | ANNVTYVWGVVSWG |
| | | | ENCGKPEFPGVYTK |
| | | | VANYFDWISYHVGR |
| | | | PFISQYNVGGSSGG |
| | | | GHCQAPDHFLFAKL |
| | | | KTQTNASDFPIGTS |
| | | | LKYECRPEYYGRPF |
| | | | SITCLDNLVWSSPK |
| | | | DVCKRKSCKTPPDP |
| | | | VNGMVHVITDIQVG |
| | | | SRINYSCTTGHRLI |
| | | | GHSSAECILSGNAA |
| | | | HWSTKPPICQRIPC |
| | | | GLPPTIANGDFIST |
| | | | NRENFHYGSVVTYR |
| | | | CNPGSGGRKVFELV |
| | | | GEPSIYCTSNDDQV |
| | | | GIWSGPAPQCII |
| | | | (SEQ ID NO: 22) |
| CR1(ccp15) + | | fH and CR1 | |
| fH(ccp2) + | | | |
| fH(ccp3) + | | | |
| fH(ccp4) | | | |
| fH(ccp1) + | | | |
| CR1(ccp16) + | | | |
| fH(ccp3) + | | | |
| fH(ccp4) | | | |
| fH(ccp1) + | | | |
| fH(ccp2) + | | | |
| CR1(ccp17) + | | | |
| fH(ccp4) | | | |
| CR1(ccp15) + | | | |
| CR1(ccp16) + | | | |
| fH(ccp3) + | | | |
| fH(ccp4) | | | |
| fH(ccp1) + | | | |
| CR1(ccp16) + | | | |
| CR1(ccp17) + | | | |
| fH(ccp4) | | | |
| CR1(ccp15) + | | | |
| fH(ccp2) + | | | |
| CR1(ccp17) + | | | |
| fH(ccp4) | | | |
| CR1(ccp15) + | | | |
| CR1(ccp16) + | | | |
| CR1(ccp17) + | | | |
| fH(ccp4) | | | |
| CR1(ccp15- | | | |
| 17) | | | |

### F. Combination Fusion Constructs

In some embodiments, provided herein are fusion constructs comprising at least one domain of complement factor I (CFI), a second component, a third component, a fourth component, and a fifth component, as provided in FIG. 7 and Table 3. These exemplary fusion constructs may comprise a combination of components fused together, and each include at least one “core” CFI domain. As noted above, some exemplary fusion constructs comprising a first component comprising CFI, a second component, and a third component may include a fusion construct comprising albumin, at least one CFI domain, and at least one domain of Factor H (FH).

FIG. 5 depicts a schematic representation of three exemplary fusion constructs comprising HSA, at least one CFI domain, and various domains of Factor H. Each of the exemplary fusion constructs shown may comprise a CFI-HSA portion comprising a leader sequence, HSA, a wild type CFI as described earlier herein, and varying domains of FH (referred to as the “CCP-part” in FIG. 5). As noted above, the CFI-HSA portion may be constructed as with a GGSSGG (SEQ ID NO: 6) linker fusing together the HSA and SPD of CFI. Exemplary fusion referred to herein as the CFI-HSA-FH_CCP1-4 fusion construct comprises a wild type CFI, and CCP domains of FH 1-4. CFI-HSA-FH_CCP1-4 also comprises a GGSSGG (SEQ ID NO: 6) linker, which is a combination of the GGSSGG-linker (SEQ ID NO: 6) and the Gly-only linker that connects together the CCP4 domain and the CCP19 domain in mini Factor H. Other exemplary fusion construct shown comprise CCP domains 2-4 of FH, and CCP domains 2-3 of FH. The lengths of the linkers used in the exemplary fusion constructs are shown, with conservative minimum lengths shown in parentheses. It should be understood that any other suitable flexible linkers may also be used.

Other exemplary fusion constructs provided herein comprise a wild type CFI or CFI variant, at least one FH domain, and at least one CR1 domain. In some embodiments, the fusion construct comprises wild type CFI or CFI variant, at least one FH domain, and at least one CR1 domain. In some embodiments, the fusion construct comprises human serum albumin, a wild type CFI or CFI variant, and at least one FH domain, and at least one CR1 domain. The fusion constructs comprising at least one FH domain and at least one CR1 domain can comprise an orientation including an FH domain fused to a CR1 domain, alternating FH and CR1 domains, one or more sequential FH domains fused to one or more sequential CR1 domains, one or more sequential CR1 domains fused to one more FH domains, or combinations thereof. In some embodiments, the fusion construct comprises a wild type CFI or CFI variant, hCR1; CCP15; CCP16; CCP17, and hFH; CCP1; CCP2; CCP3; CCP4. In some embodiments, the fusion construct comprises a wild type CFI or CFI variant and hCR1; CCP15; hFH; CCP2; CCP3; CCP4. In some embodiments, the fusion construct comprises a wild type CFI or CFI variant and hFH; CCP1; hCR1; CCP16; hFH; CCP3; CCP4. In some embodiments, the fusion construct comprises a wild type CFI or CFI variant and hCR1; CCP15; CCP16; hFH; CCP3; CCP4. In some embodiments, the fusion construct comprises a wild type CFI or CFI variant and hFH; CCP1; hCR1; CCP16; CCP17; hFH; CCP4. In some embodiments, the fusion construct comprises a wild type CFI or CFI variant and hCR1; CCP15; CCP16; CCP17; hFH; CCP4. It is understood that any of the fusion constructs may further comprise one or more linkers as described herein. In some embodiments, the fusion construct comprises a wild type CFI or CFI variant, at least one FH domain, at least one CR1 domain, and a linker region. It is understood that any of the fusion constructs may further comprise a human serum albumin. In some embodiments, the fusion construct comprises a human serum albumin, a wild type CFI or CFI variant, at least one FH domain, and at least one CR1 domain.

In some embodiments, provided herein are fusion constructs comprising a first component comprising at least one CFI domain, a second component, and a third component, wherein the second component is at least one domain of FH, and the third component is any half-life extender. In some embodiments, the third component is a protein (e.g. serum albumin or Fc). In some embodiments, the third component is not a protein (e.g. PEG).

### II. Generation of CFI Variants and CFI Fusion Constructs

Provided herein are methods and compositions for generating CFI variants and CFI fusion constructs. Accordingly provided are nucleic acids and vectors encoding any of the CFI variants or fusion constructs of the disclosure. Also provided are cells comprising one or more nucleic acids encoding a CFI or variant thereof, and fusion constructs of the disclosure.

Provided herein are nucleic acids encoding the CFI variants and fusion constructs described herein.

Provided herein are expression vectors encoding the CFI variants and fusion constructs described herein. Expression vectors can include transcription regulatory elements, such as enhancers or promoters, operably linked to the nucleic acid sequence encoding the CFI variant or fusion construct of the disclosure.

Cell lines can be developed to express production of the CFI and the variants and fusion constructs described herein. Cell lines for producing CFI, CFI can be accomplished using any host cell capable of expressing the CFI variants, and CFI fusions constructs described herein. Host cells can be mammalian cells, insect cells, fungal cells, plant cells, and/or bacterial cells. For expression of the CFI variants and fusion constructs, the host cell line can be transiently or stably transfected or transduced with expression vectors encoding the CFI, CFI variants, and CFI fusions. Vectors can be, for example, plasmids or viral vectors. In some embodiments, the host cell line is a mammalian cell line. In some embodiments, the host cell is a Chinese hamster ovary (CHO) cell.

CFI variants and fusion constructs described herein can be recombinantly expressed in mammalian cell lines known in the art for producing biologic products, e.g. Chinese hamster ovary (CHO) cells. Mammalian cells can be transfected or transduced with an expression vector encoding the CFI variants and fusion constructs described herein using any method known in the art.

Provided herein are methods of generating a CFI or a variant thereof in an activated state; the method comprising producing the CFI in a cell comprising one or more nucleic acid encoding the CFI or variant thereof, and an expression cassette for furin.

Provided herein are methods for production and purification of CFI variants and fusion constructs described herein. CFI variants and fusion constructs described herein may be purified from conditioned media by standard methods known in the art. In some embodiments CFI variants and fusion constructs may be purified by chromatography on affinity matrices. In some embodiments the affinity matrix is CaptureSelect™ human albumin affinity matrix. In some embodiments CFI variants and fusion constructs may be purified by chromatography on cation and/or anion exchange matrices and optionally size exclusion chromatography. CFI variants and fusion constructs may optimally be buffer exchanged into any suitable buffer known in the art. Purity can be assessed by any method known in the art including gel electrophoresis, orthogonal HPLC methods, staining and spectrophotometric techniques.

### III. Uses of CFI Variants and CFI Fusion Constructs

The CFI variants and fusion constructs of the disclosure may be used for modulating the complement system.

As discussed herein, in some embodiments, a CFI variant or CFI fusion construct of the disclosure is capable of modulating the classical and lectin complement pathway.

As discussed herein, in some embodiments, a CFI variant or CFI fusion construct of the disclosure is capable of modulating the alternate complement pathway.

As discussed herein, in some embodiments, a CFI variant or CFI fusion construct of the disclosure is capable of decreasing the amplification of the complement system.

As discussed herein, in some embodiments, a CFI variant or CFI fusion construct of the disclosure is capable of increasing the cleavage of C3b.

As discussed herein, in some embodiments, a CFI variant or CFI fusion construct of the disclosure is capable of increasing the cleavage of C4b.

As discussed herein, in some embodiments, a CFI variant or CFI fusion construct of the disclosure is capable of increasing the generation of C4c.

As discussed herein, in some embodiments, a CFI variant or CFI fusion construct of the disclosure is capable of increasing the generation of iC3b.

As discussed herein, in some embodiments, a CFI variant or CFI fusion construct of the disclosure is capable of increasing the generation of C3dg from iC3b.

As discussed herein, in some embodiments, a CFI variant or CFI fusion construct of the disclosure is capable of increasing the generation of C3c from iC3b.

As discussed herein, in some embodiments, a CFI variant or CFI fusion construct of the disclosure is capable of reducing the level of C3b α-chain.

As discussed herein, in some embodiments, a CFI variant or CFI fusion construct of the disclosure is capable of increasing the hydrolysis of a peptide substrate.

As discussed herein, in some embodiments, a CFI variant or CFI fusion construct of the disclosure is capable of increasing the proteolysis of a macromolecular protein substrate.

As discussed herein, in some embodiments, a CFI variant or CFI fusion construct of the disclosure is capable of reducing in the level or function of membrane attack complex (MAC).

As discussed herein, in some embodiments, a CFI variant or CFI fusion construct of the disclosure is capable reducing observed hemolysis.

As discussed herein, in some embodiments, a CFI variant or CFI fusion construct of the disclosure is capable of increasing the cleavage of C3b in the absence of cofactor, e.g. in a cofactor independent manner.

As discussed herein, in some embodiments, a CFI variant or CFI fusion construct of the disclosure is capable of increasing the cleavage of C4b in the absence of cofactor, e.g. in a cofactor independent manner.

The CFI variants and fusion constructs of the disclosure may be used for therapeutics in a subject. As used herein, a subject includes any mammalian subject and includes primates, rodents, domestic animals, zoo animals, and pets. In some embodiments, the mammalian subject is a human subject. In some embodiments, the mammalian subject is a non-human primate.

### A. CFI Variants and Fusion Constructs for Modulation of the Complement System

Provided herein is a method of modulating the complement system, comprising contacting a sample in vitro or a tissue in vivo with any one of the CFI variants or fusion constructs provided herein. In some embodiments, the sample is plasma.

### B. Treatment of Non-Ocular Conditions

In some embodiments, the CFI variants or fusion constructs provided herein are useful for treating a non-ocular condition in a subject. In some embodiments, provided herein is a method of treating an ocular condition in a subject in need thereof, the method comprising administering to the subject a therapeutically effective amount of any one of the CFI variants or fusion constructs provided herein, or the pharmaceutical composition provided herein below.

In some embodiments, the non-ocular condition is characterized by a deficiency of CFI. In some embodiments, the non-ocular condition is characterized by dysregulation of the complement system.

In some embodiments, the non-ocular condition is a systemic acute indication. In some embodiments, the non-ocular condition is a systemic acute indication selected from the group consisting of: acute glomerulonephritis, acute renal injury, acute respiratory distress syndrome, bacterial meningitis, brain hemorrhage, burns, coronavirus infection, Epstein-Barr virus infection, hematopoietic stem cell transplantation, ischemia reperfusion injury, Lyme disease, myocardial infarction, organ transplantation, periodontitis, pneumonia, pre-eclampsia, schistosomiasis, sepsis, stroke, thromboembolism, and traumatic brain injury.

In some embodiments, the non-ocular condition is a systemic chronic indication. In some embodiments, the non-ocular condition is a systemic chronic indication selected from the group consisting of: Alzheimer's disease, anti-neutrophil cytoplasmic antibody (ANCA)-associated vasculitis, antiphospholipid syndrome, asthma, atherosclerosis, atypical hemolytic uremic syndrome (aHUS), autoimmune hemolytic anemia, bullous pemphigoid (BP), C3 glomerulopathy, chronic kidney failure, chronic obstructive pulmonary disease (COPD), Cold agglutinin disease (CAD), Crohn's disease, diabetic neuropathy, generalized myasthenia gravis (gMG), Granulomatosis with Polyangiitis (GPA), Guillain-Barré Syndrome (GBS), hereditary angioedema (HAE), hidradenitis suppurativa (HS), IgA nephropathy (IgAN), lupus nephritis (LN), membranous glomerulonephritis (MN), microscopic polyangiitis (MPA), motor neuron disease, multifocal motor neuropathy (MMN), multiple sclerosis (MS), non-insulin dependent diabetes, osteoarthritis, pancreatitis, Parkinson's disease, paroxysmal nocturnal hemoglobinuria (PNH), post-transplant lymphoproliferative disease, protein losing enteropathy, psoriasis, pyoderma gangrenosum, rheumatoid arthritis, schizophrenia (SZ), systemic lupus erythematosus (SLE), immune thrombocytopenia (ITP), and ulcerative colitis, Lampert-Eaton myasthenic syndrome (LEMS), CHAPLE syndrome (CD55 deficiency), thrombotic microangiography (TMA) and chronic inflammatory demyelinating polyneuropathy (CIDP), Huntington disease and ischemia reperfusion injuries.

In some embodiments, the CFI variants or fusion constructs provided herein have an improved characteristic as compared to a wild type CFI. In some embodiments, the improved characteristic is an increase in activity, wherein the increase in activity comprises an increase in the cleavage of C3b and/or C4b. The potency and specificity of the CFI variant provided herein can be tuned for particular therapeutic indications. In some embodiments, the CFI variants or fusion constructs provided herein are C3b degraders. In some embodiments, the C3b degraders are useful for the treatment of diseases. In some embodiments, the CFI variants provided herein are C4b degraders and are useful for the treatment of diseases. For example, the diseases that may be treated by use of the C4b degraders include, but are not limited to a non-ocular condition. In some embodiments, the non-ocular condition is a systemic chronic indication. In some embodiments, the non-ocular condition is a systemic chronic indication selected from the group consisting of: Alzheimer's disease, Amyotrophic lateral sclerosis (ALS), anti-neutrophil cytoplasmic antibody (ANCA)-associated vasculitis, antiphospholipid syndrome, asthma, atherosclerosis, atypical hemolytic uremic syndrome (aHUS), autoimmune hemolytic anemia, bullous pemphigoid (BP), C3 glomerulopathy, chronic kidney failure, chronic obstructive pulmonary disease (COPD), Cold agglutinin disease (CAD), Crohn's disease, diabetic neuropathy, generalized myasthenia gravis (gMG), Granulomatosis with Polyangiitis (GPA), Guillain-Barré Syndrome (GBS), hereditary angioedema (HAE), hidradenitis suppurativa (HS), IgA nephropathy, lupus nephritis (LN), membranous glomerulonephritis (MN), microscopic polyangiitis (MPA), motor neuron disease, multifocal motor neuropathy (MMN), multiple sclerosis (MS), non-insulin dependent diabetes, osteoarthritis, pancreatitis, Parkinson's disease, paroxysmal nocturnal hemoglobinuria (PNH), post-transplant lymphoproliferative disease, protein losing enteropathy, psoriasis, pyoderma gangrenosum, rheumatoid arthritis, schizophrenia (SZ), systemic lupus erythematosus (SLE), immune thrombocytopenia (ITP), warm Autoimmune hemolytic anemia (wAIHA), Immune-Complex Membranoproliferative Glomerulonephritis (IC-MPGN), and ulcerative colitis, Lampert-Eaton myasthenic syndrome (LEMS), CHAPLE syndrome (CD55 deficiency), thrombotic microangiography (TMA) and chronic inflammatory demyelinating polyneuropathy (CIDP). Huntington disease and ischemia reperfusion injuries.

In some embodiments, the non-ocular condition is non-oncological.

In some embodiments, the non-ocular condition is oncological. In some embodiments, the non-ocular condition is oncological, and is characterized by solid tumors, or by liquid tumors. In some embodiments, the non-ocular condition is characterized by solid tumors, and is selected from the group consisting of: colorectal tumors, hormone-refractory prostate cancer, melanoma, metastatic breast cancer, metastatic colorectal cancer, metastatic esophageal cancer, metastatic pancreas cancer, metastatic stomach cancer, nasopharyngeal carcinoma, non-small cell lung cancer, pancreas tumors, squamous cell carcinoma, and stomach tumors. In some embodiments, the non-ocular condition is characterized by liquid tumors, and is selected from the group consisting of: acute myelogenous leukemia, B-cell lymphoma, and Hodgkin's disease.

### C. CFI Variants and Fusion Constructs for Treatment of Ocular Conditions

In some embodiments, the CFI variants or fusion constructs provided herein are useful for treating an ocular condition in a subject. In some embodiments, provided herein is a method of treating an ocular condition in a subject in need thereof, the method comprising administering to the subject a therapeutically effective amount of any of the CFI variants or fusion constructs provided herein, or the pharmaceutical composition provided herein below.

In some embodiments, the ocular condition is characterized by a deficiency of CFI. In some embodiments, the ocular condition is characterized by dysregulation of the complement system.

In some embodiments, the ocular condition is characterized by the presence of a dysfunctional CFI gene. In some embodiments, the ocular condition is characterized by dysregulation of the complement system and low CFI levels.

In some embodiments, the ocular condition selected from the group consisting of: diabetic macular edema (DME), diabetic retinopathy, dry age-related macular degeneration (AMD), glaucoma, keratoconjunctivitis, neuromyelitis optica spectrum disorder (NMOSD), open angle glaucoma, polypoidal choroidal vasculopathy, Stargardt Disease, uveitis, and vitreoretinopathy.

In some embodiments, wherein the ocular condition is non-oncological.

### D. Combination Therapies

The administration of any one of the therapeutic CFI variants or fusion constructs provided herein may be a monotherapy, or may be in combination with any other known drugs or treatments. The other known drugs or treatments may be for conditions associated with dysregulation of the complement system, or may be associated with a CFI deficiency. In some embodiments, the conditions may be ocular. In some embodiments, the conditions may be non-ocular. In some embodiments, the therapeutic CFI variants or fusion constructs provided herein are co-administered with one or more C5 inhibitors. In some embodiments, the C5 inhibitor is eculizumab. In some embodiments, the C5 inhibitor is cemdisiran.

### E. Administration

The CFI variants and fusion constructs described herein may be delivered as polypeptide-based therapies, or nucleic-acid based therapies.

Such treatment as contemplated herein includes both administration of a CFI variant of the disclosure or fusion construct of the disclosure, as well as administration of one or more nucleic acids encoding for a CFI variant of the disclosure or a fusion construct of the disclosure. Accordingly, provided herein are pharmaceutical compositions comprising the CFI variants of the disclosure, CFI fusion constructs of the disclosure, as well as pharmaceutical compositions comprising one or more nucleic acids encoding for CFI variants of the disclosure and encoding for fusion constructs of the disclosure.

Accordingly provided herein are nucleic acids encoding the CFI variants and fusions constructs of the disclosure and are delivered as a part of a nucleic acid-based gene therapy to a subject in need. In some embodiments, the nucleic acid encoding for a CFI variant or fusion construct of the disclosure is delivered as a part of a viral vector based gene therapy (e.g. lentiviral-based therapy, adenoviral-based therapy, adeno-associated viral-based therapy, and the like). In some embodiments, the nucleic acid encoding for a CFI variant or fusion construct of the disclosure is delivered as a naked nucleic acid. In some embodiments, the nucleic acid encoding for a CFI variant or fusion construct of the disclosure is delivered inside a liposome. In some embodiments, the nucleic acid encoding for a CFI variant or fusion construct of the disclosure is delivered as a part of a nanoparticle. In some embodiments, the nucleic acid encoding for a CFI variant or fusion construct of the disclosure is delivered as a part of a virus-like particle.

In some embodiments, the CFI variants and fusion constructs described herein may be delivered as polypeptide-based therapeutics.

The in vivo administration of the therapeutic CFI variants or fusion constructs described herein (protein or nucleic acid based therapeutics) may be carried out intravenously, intramuscularly. subcutaneously, topically, orally, transdermally, intraperitoneally, intraorbitally, intrathecally, intraventricularly, intranasally, transmucosally, through implantation, or through inhalation. Administration of the therapeutic fusion constructs may be performed with any suitable excipients, carriers, or other agents to provide suitable or improved tolerance, transfer, delivery, and the like.

In exemplary embodiments, administration of the therapeutic CFI variants or fusion constructs described herein is a subcutaneous administration. In some embodiments, the subcutaneous administration is a daily, every other day, twice weekly, or weekly administration.

In some embodiments, administration of the therapeutic CFI variants or fusion constructs described herein is an intravenous administration.

As generally contemplated herein, the CFI variants or fusion constructs described herein are delivered in an activated two chain form. However, in some instances, inactive CFI variants or fusion constructs can be delivered in an inactive single chain form. In some embodiments, what is delivered comprises both single chain inactive and two chain active forms.

### F. Dosages

In some embodiments, any of the therapeutic CFI variants or fusion constructs described herein may be administered to a subject in need thereof in a dosage of about 0.05 mg/kg to about 10 mg/kg. In some embodiments, the dosage is about 1 mg/kg. In some embodiments, administration of the therapeutic CFI variants or fusion constructs described herein is a subcutaneous administration, at a dosage of about 0.05 mg/kg. 0.1 mg/kg, about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, or about 10 mg/kg. In some embodiments, administration of the therapeutic CFI variants or fusion constructs described herein is an intravenous administration, at a dosage of about 0.05 mg/kg, 0.1 mg/kg, about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, or about 10 mg/kg. In some embodiments, administration of the therapeutic CFI variants or fusion constructs described herein is daily administration, every other day administration, weekly administration, or twice weekly administration.

In some embodiments, the target level of the therapeutic fusion constructs in plasma may be about 0.05 mg/kg. 0.1 μg/ml, about 0.5 μg/ml, about 1 μg/ml, about 1.5 μg/ml, about 2 μg/ml, about 2.5 μg/ml, about 3 μg/ml, about 3.5 μg/ml, about 4 μg/ml, about 4.5 μg/ml, 5 μg/ml, about 5.5 μg/ml, about 6 μg/ml, about 6.5 μg/ml, about 7 μg/ml, about 7.5 μg/ml, about 8 μg/ml, about 8.5 μg/ml, about 9 μg/ml, about 9.5 μg/ml, about 10 μg/ml, about 10.5 μg/ml, about 11 μg/ml, about 11.5 μg/ml, about 12 μg/ml, about 12.5 μg/ml, about 13 μg/ml, about 13.5 μg/ml, about 14 μg/ml, about 14.5 μg/ml, 15 μg/ml, about 15.5 μg/ml, about 16 μg/ml, about 16.5 g/ml, about 17 μg/ml, about 17.5 μg/ml, about 18 μg/ml, about 18.5 μg/ml, about 19 μg/ml, about 19.5 μg/ml, about 20 μg/ml, about 20.5 μg/ml, about 21 μg/ml, about 21.5 μg/ml, about 22 μg/ml, about 22.5 μg/ml, about 23 μg/ml, about 23.5 μg/ml, about 24 μg/ml, about 24.5 μg/m,. 25 μg/ml, about 25.5 μg/ml, about 26 μg/ml, about 26.5 μg/ml, about 27 μg/ml, about 27.5 μg/ml, about 28 μg/ml, about 28.5 μg/ml, about 29 μg/ml, about 29.5 μg/ml, about 30 μg/ml. The target level may be about 10 μg/ml, about 25 μg/ml, about 50 μg/ml, about 100 μg/ml, about 150 μg/ml, about 200 μg/ml, about 250 μg/ml, or even about 300 μg/ml. Exemplary fusion constructs that may be administered to a subject in need thereof to achieve a target level of about 20 μg/ml may include CFI-HSA, comprising a CFI corresponding to a wild type CFI.

### G. Formulations

Pharmaceutical compositions containing a CFI variant or fusion constructs of the disclosure can be formulated in any conventional manner by mixing a selected amount of the polypeptide with one or more physiologically acceptable carriers or excipients, for use in the treatments provided herein. Selection of the carrier or excipient is within the skill of the administering profession and can depend upon a number of parameters. These include, for example, the mode of administration and disorder treated. The pharmaceutical compositions provided herein can be formulated for single dosage (direct) administration or for dilution or other modification. The concentrations of the compounds in the formulations are effective for delivery of an amount, upon administration, that is effective for the intended treatment. Typically, the compositions are formulated for single dosage administration, but not necessarily.

### H. Pharmaceutical Compositions

The disclosure also provides pharmaceutical compositions comprising any one of the CFI variants or fusion constructs disclosed herein, and optionally a pharmaceutical acceptable excipient or carrier. In some embodiments, the pharmaceutical composition is sterile. The pharmaceutical compositions may be formulated to be compatible with their intended routes of administration. In some embodiments, the pharmaceutical compositions of the disclosure are suitable for administration to a human subject, or other non-human primate. In exemplary embodiments, the pharmaceutical composition is formulated for subcutaneous administration.

### I. Kits and Articles of Manufacture for Therapeutic CFI Variants and Fusion Constructs

The disclosure also provides a kit or article of manufacture comprising any one of the CFI variants or fusion constructs disclosed herein, or any pharmaceutical composition disclosed herein. In some embodiments, the kits may further include instructional materials for carrying out any of the methods disclosed herein. In some embodiments, the kits may further include sterile containers or vials for holding the fusion constructs and/or pharmaceutical compositions disclosed herein. In some embodiments, the kits may further include sterile delivery devices for administering the fusion constructs and/or pharmaceutical compositions disclosed herein. In some embodiments, an article of manufacture comprises any pharmaceutical composition of the disclosure.

### EXAMPLES

### Example 1: Expression, Purification, Activation, and In Vitro Sialylation of CFI Variants and Fusion Proteins

### Overview

For Example 1, reference to CFI-HSA refers to human serum albumin fused to the N-terminal end of a human wild type CFI (SEQ ID NO: 21).

A wild type CFI-HSA protein is expressed in Chinese hamster ovary (CHO) cells, purified with anti-albumin affinity purification, activated with furin, and purified by sizing columns. The activated CFI-HSA protein was subjected to in vitro sialylation to increase the total sialylation of CFI-HSA. Finally, the sialylated protein was purified using anti-albumin affinity purification and polished by size-exclusion column chromatography.

### Expression

The CFI-HSA gene (SEQ ID NO: 21) was synthesized (ThermoFisher Scientific, Geneart, Regensburg, Germany), with the human serum albumin at the amino terminus of the CFI protein. The protein was made with the signal sequence of SEQ ID NO: 2, which was removed during expression. The amino terminal albumin tag was connected to the CFI gene through a linker (SEQ ID NO: 6). The gene of CFI-HSA was inserted into an expression vector (Lake Pharma, Hayward, CA) utilizing standard molecular biology techniques. The resulting plasmid DNA was transformed into E. coli. The transfected E. coli were grown in 200 ml of LB media for expression of plasmid DNA and harvested utilizing standard techniques. The plasmid DNA was run on an agarose gel for quality assessment and sequence confirmed before proceeding to transfection.

1.0 liter of suspension TunaCHO™ cells were seeded in a shake flask and were expanded using serum-free chemically defined medium. On the day of transfection, the expanded cells were seeded into a new flask with fresh medium. The plasmid DNA was transiently transfected into the CHO cells using Lipofectamine 2000 (ThermoFisher Scientific). The cells were maintained as a batch-fed culture until the end of the production run. The protein was expressed for 14 days at 37° C. at 125 RMP with 8% CO₂ concentration. Cells were centrifuged and supernatant was collected for purification of secreted CFI-HSA at the end of 14 days expression.

### Purification

The supernatant with expressed CFI-HSA protein was passed through a 10 ml gravity flow column of CaptureSelect™ human albumin affinity matrix (ThermoFisher Scientific). Column-bound protein was washed with 10 column volume of 20 mM sodium phosphate buffer. Bound CFI-HSA protein was eluted in two steps: first, with 3 column volume of 20 mM Tris-HCl, pH 7.0 buffer with and 2 M MgCl2, and second, with 3 column volume of 20 mM citric acid, pH 3.0. Elution from both steps 1and 2 was collected in 5 ml fractions. Each fraction of the step 2 elution was neutralized with 10% of neutralization buffer (1.5 M tris-HCL pH 7.4). All fractions were analyzed by reducing and non-reducing SDS-PAGE electrophoresis and bands were visualized by SimplyBlue™ SafeStain (ThermoFisher Scientific). CFI-HSA runs as a 130 kDa band on a non-reducing gel and as 102 kDa and 28 kDa bands on a reducing gel. Fractions with maximum CFI-HSA concentration and purity were pooled for further processing.

### Furin Activation

CFI-HSA is expressed as an inactive, single chain precursor protein, and is activated by furin, another serine protease. Furin is an endoprotease that cleaves CFI at its conserved RRKR sequence (also referred to as the furin recognition sequence), resulting in a heavy and light chain connected by a disulfide bond. The furin-processed, mature, two-chain protein is the activated form of the CFI protein.

Cleavage of CFI-HSA for producing the protein in its activated form was performed by incubation of 4 μg of recombinant furin per mg of purified CFI-HSA in Tris-NaCl (tris buffered saline), 2.5 mM CaCl, and 0.5% CHAPS at 30° C. for 18 hours. The CFI-HSA protein concentration was maintained at 1.4 mg/ml. This results in more than 90% activation of the protein. The activated protein was separated from inactivated CFI-HSA, and other proteins by size-exclusion chromatography. Size exclusion chromatography (SEC) was performed using a HiLoad 16/600 Superdex 200 column (GE Healthcare Life Sciences) and phosphate buffer saline (PBS, 137 mM NaCl, 2.7 mM KCI, 10 mM Na₂HPO₄, 2 mM KH₂PO₄, pH 7.4) as the mobile phase. Collected fractions were analyzed by CE-SDS (LabChip GXII, Perkin Elmer). Fractions containing the target protein were pooled and analyzed by SE-UPLC.

### In Vitro Sialylation

The activated CFI-HSA protein was subject to in vitro sialylation. Briefly, the sialylation was carried out in a two-step enzymatic reaction. First, a galactosylation reaction of CFI-HSA was performed in a 200 μl volume utilizing a 1:200 molar ratio of galactosyltransferase (GalT1) enzyme and CFI-HSA in 10 mM UDP-Galactose, 5 mM MnCl2, and 100 mM MES, pH 6.5 buffer. Galactosylated CFI-HSA was purified from the reaction mixture by CaptureSelect™ Human Albumin affinity chromatography, as described earlier. Next, the sialylation reaction was performed in a 250 μl volume utilizing a 1:50 molar ratio of enzyme alpha 2,6-sialyl transferase and purified CFI-HSA in 80 μM Alkaline phospahatase, 6.1 mM CMP-NANA, 10 mM ZnCl, and 200 mM MES buffer, pH 6.5 at 37° C. for 1 hour. The sialylated CFI-HSA protein was purified from the reaction mixture by CaptureSelect™ Human Albumin affinity chromatography. The extent and characteristics of the sialic acid chain on CFI-HSA was determined by utilizing an Agilent/Prozyme Analytical service, GS-SAP method for total sialic acid quantitation (Agilent GS48), and mass spectrophotometric (MS) analysis (Lake Pharma analytical service), described in further detail below.

Briefly, total sialic acid quantitation was performed by mixing 20 μl of each sample with 10 μl of release reagent in a 96 well plate. The reaction mixture was incubated for 2 hours at 80° C. The samples were cooled to room temperature and 10 μl of labeling reagent was added to each sample for a further incubation of 3 hours at 50° C. The samples were again cooled down to room temperature and 160 μl of de-ionized (dI) water was added to bring the total volume to 200 μl. 10 μl of sample was injected in the Agilent UHPLC Poroshell C18 column to run at a flow rate of 0.4 ml/minute at 30° C. in 4% methanol, 8% acetonitrile in water (Line Al) and 100% ACN (Line B1). The peaks were recorded at 373/448 nm wavelength. A standard curve of total peak area versus picomoles (pmol) of sialic acid was generated by running 1-2000 pmol of NANA (N-acetylneuraminic acid, Neu5Ac) supplied with the kit on the same column. Total sialic acid of each sample was quantitated by comparing the peak area of samples against the standard curve.

The mass spectrometric analysis was performed by a standard trypsin Q-TOF mass spectrometer. Briefly, all samples were treated, reduced and alkylated by DTT and iodoacetamide, followed by trypsin digestion. The digested samples were analyzed by Waters ACQUITY UPLC coupled to a Xevo G2-XS-QTOF mass spectrometer using a protein BEH C18 column.

### Polishing

Purified CFI-HSA protein was subjected to size-exclusion chromatography (SEC) using a HiLoad 16/600 Superdex 200 column (GE Healthcare Life Sciences) and phosphate buffer saline as the mobile phase. Collected fractions were analyzed by CE-SDS (LabChip GXII, Perkin Elmer). Fractions containing the target protein were pooled, and the concentration was brought to 5 mg/ml, and the samples were flash frozen for storage at −80° C.

### Expression and Purification of CFI-HSA Variants

The DNA of CFI-HSA variants was generated either by synthesis or by site-directed mutagenesis utilizing standard techniques. The proteins were expressed in 250 ml of suspension in TunaCHO™ cells, as described herein with reference to wild type CFI-HSA protein, with the exception that the expression was done for 7 days instead of 14 days. After 7 days, the cells were centrifuged, and conditioned media was passed through a gravity flow column of CaptureSelect™ human albumin affinity matrix (ThermoFisher Scientific). Column-bound protein was washed with 10 column volume of 20 mM sodium phosphate buffer. Bound CFI-HSA protein was eluted with 3 column volume of 20 mM Tris-HCl, pH 7.0 buffer with and 2 M MgCl, in 5 ml fractions. CFI-HSA or its variants were buffer exchanged (either by dialysis or a spin concentrator) into 30 mM HEPES, 150 mM NaCl, 2.5 mM CaCl₂, pH 7.4. Recombinant human furin, at a molar ratio of 1:25 (furin:CFI-HSA), was added to CFI-HSA and the reaction mixture was incubated at 30° C. for 16 hours. Two micrograms of the activation mixture was run on a 9% SDS-PAGE gel to assess the activation efficiency. Generally, more than 80% activation was achieved.

### N-Terminal Albumin Fusion Provides Solubility and Facilitates Activation of CFI-HSA

Activation of the CFI variants and fusion proteins comprising such variants of the disclosure can be compared to wild type CFI or to each other. To do so, a gene construct for WT-CFI was expressed essentially as described above for CFI-HSA. The purity of the recombinant protein can be assessed by SDS-PAGE under reduced and non-reduced conditions, in either the presence or absence of furin. It has been previously shown in PCT application no. PCT/US2021/037278 that there is a significant and unexpected benefit of the N-terminal HSA tag (in SEQ ID NO: 21) for maintaining solubility, monodispersity and efficient furin activation. The same can be assessed for the CFI variants and fusion proteins of the disclosure.

### Example 2: Characterization by Peptidolytic Activity Assay

For Example 2, reference to CFI-HSA refers to human serum albumin fused to the N-terminal end of wild type CFI (SEQ ID NO: 21).

The proteolytic activity of wild type CFI-HSA and CFI variant-HSA fusions (referred collectively herein as “CFI-HSA proteins”) can be tested by following the cleavage of chromogenic substrates by use of a chromophore. The S-2288 (Chromogenix) peptide substrate is selected for this assay as it is sensitive to a broad spectrum of serine proteases. The peptidolytic activity of the CFI-HSA proteins are measured by the rate of generation of p-nitroaniline (pNA) upon substrate cleavage, which occurs spectrophotometrically at 405 nm.

The CFI-HSA proteins are diluted to an initial concentration of 400 nM in 100 μl of HBS/BSA (30 mM HEPES, 140 mM NaCl, 0.2% BSA, pH 7.4) in a non-coated 96-well microplate (Nunc). A working stock of 4 mM S-2288 is made in HBS/BSA in a separate tube. The microplate and diluted chromogenic substrate are pre-warmed to 37° C. for 5 minutes. The assay is initiated by the addition of 100 μl of pre-warmed S-2288 to the wells of the microplate containing the CFI-HSA proteins. This results in a final concentration of 200 nM of the CFI-HSA proteins, and 2 mM of S-2288 substrate in a 200 μl reaction volume. The rate of substrate cleavage is recorded every 30 seconds for 3 hours at 37° C. at 405 nm, using a microplate reader (Multiskan™ GO Microplate Spectrophotometer, Thermo Scientific). Peptide hydrolysis activity of wild type CFI-HSA is normalized as 100% in order to calculate the percentage of peptidolysis activity of the CFI-HSA variants.

### Example 3: Characterization by a C3b Cleavage Assay

For Example 3, reference to CFI-HSA refers to human serum albumin fused to the N-terminal end of wild type CFI (SEQ ID NO: 21).

The C3b cleavage assay is a functional assay that can be used to determine the ability of wild type CFI, wild type CFI-HSA and variant CFI, and CFI-HSA variants (referred collectively herein as “CFI-HSA proteins”) to cleave its natural substrate, C3b. An exemplary protocol is provided herein. The CFI-HSA proteins are incubated with C3b and a truncated Factor H (mini FH) at 37° C. for analysis of C3b cleavage. Mini FH has been previously shown to be functionally active and support the CFI-mediated C3b cleavage (J Immunol. 2013 Jul. 15; 191(2):912-21). The cleavage of C3b into smaller fragments is then monitored over time by SDS-PAGE.

First, for each CFI-HSA variant, the master reaction mixture is set up at room temperature containing the final concentrations of 500 nM of mini FH and 5 nM of the CFI-HSA proteins in HBS buffer (30 mM HEPES, 140 mM NaCl pH 7.4). The master reaction mixtures are transferred to 37° C. and allowed to equilibrate for 5 minutes. The cleavage reaction is initiated by the addition of C3b to a final concentration of 0.5 μM. 20 μl samples from the master mixtures are withdrawn for each time point measured, and quenched by the addition of 5×SDS reducing sample buffer. Samples are run on a 9% SDS-PAGE gel and C3b cleavage is visualized by Coomassie staining. The amount of C3b cleavage that occurred is quantitated by densitometry. The C3b cleavage activity of wild type CFI-HSA is normalized as 100% in order to calculate the percentage of C3b cleavage activity of the CFI-HSA variants.

To compare the rate of C3b cleavage by each CFI-HSA variant to that of the wild type CFI-HSA, a time course for C3b cleavage by the CFI-HSA proteins can be performed in parallel. If disappearance of a C3 (alpha) band at a molecular weight of 114 kDa is observed (in SDS-PAGE), it is an indication of C3b cleavage. This is because C3b includes two chains, (alpha) and beta. Densitometry of the relevant stained band, corrected for the average background staining (lane intensity outside the band), can be performed to quantitatively assess.

The apparent rate for loss of band intensity can be estimated by fitting a simple exponential decay formula to the band intensity data as a function of time, thereby extracting an apparent rate constant (k) of C3b cleavage. The relative rate of C3b cleavage by the CFI-HSA variants is calculated by dividing with the corresponding WT rate: k (variant)/k (WT control). This procedure is performed on multiple independent SDS-PAGE experiments and the average of k (variant)/k (WT control) is calculated along with the accompanying standard deviation.

The percentage of the C3b alpha-chain remaining after incubation over time is measured to evaluate activity of the tested CFI variant in comparison to wild type CFI. Because even subtle differences in C3b cleavage can cause disease, such as atypical hemolytic uremic syndrome (aHUS), variants that exhibit differences can be useful for increasing activity of the complement system to counter C3-induced diseases.

### Example 4: Quantitative Analysis of CFI-HSA C3b Cleavage Activity by Measurement of C3dg Formation by Time-Resolved Immuno-fluorometric Assay (TRIFMA)

For Example 4, reference to CFI-HSA refers to human serum albumin fused to the N-terminal end of wild type CFI (SEQ ID NO: 21).

A C3dg assay can be used to determine the cleavage of C3b caused by Complement Factor I (CFI), or variants, or fusion proteins thereof. The formation of C3dg can be used as a quantitative analysis of CFI-HSA C3b cleavage activity and in one possible assay, is measured by a time-resolved immuno-fluorometric assay (TRIFMA). An exemplary protocol is provided herein.

The complement pathway in human serum is activated by using heat-aggregated IgG. The effect of plasma-derived CFI or CFI-HSA proteins, including CFI-HSA variants, on C3b cleavage is measured by capturing C3dg, utilizing a C3dg antibody on a microtiter plate. Bound C3dg is detected by a combination of a biotinylated C3dg antibody and Europium-labelled streptavidin, and measured by time-resolved fluorometry.

MaxiSorb microtiter plates (Nunc) are coated with 100 μl monoclonal IgM rat anti-human C3dg antibody at 2 μg/ml in 15 mM Na₂CO₃, 35 mM NaHCO₃, pH 9.6 coating buffer by overnight incubation at room temperature. The remaining protein binding sites are blocked by incubation with HSA at 1 mg/ml in TBS. Unbound HSA is washed with TBS-Tween.

Test samples are diluted in a 1 to 6 dilution of human serum to desired concentrations in a 100 μl volume with dilution buffer (0.14 M NaCl, 10 mM Tris, 14 mM sodium azide, with 0.05% (v/v) Tween 20 (TBS/Tween), 1 mg/ml HSA and 0.1 mg/ml of heat aggregated IgG. Four-fold, six point dilutions are made for each CFI-HSA variant to cover the variants concentration range from 3132 nM to 3 nM. The reaction mixture is incubated at 37° C. for 90 minutes and quenched by 10 mM EDTA. To capture the generated C3dg. 100 μl of each reaction mixture are added to the antibody-coated microtiter wells and incubated overnight at 4° C. To detect the bound C3dg, 100 μl of biotinylated rabbit anti-C3dg antibody (DAKO) is added at 0.5 μg/ml to the wells and incubated for 2 hours at room temperature. After washing with the Eu3+-streptavidin combination (Perkin Elmer), 25 μM EDTA is added to the wells and incubated for 1 hour at room temperature ( 1/1000). After washing, 200 μl enhancement buffer (Ampliqon) is added to each well. Plates are read using a DELFIA-reader Victor5+ (Perkin Elmer) by time-resolved fluorometry.

### Example 5: Characterization by a Hemolysis Assay

A hemolysis assay can be used for the measurement of hemolytic function of a compound that uses the complement pathway. Complement Factor I (CFI) mediates C3b cleavage with its cofactor Factor H (FH) within the alternative pathway of the complement pathway system. To test the hemolytic function in the alternative pathway, C3-deficient human serum spiked with human C3 is incubated with variants/fusion proteins of the disclosure and rabbit Alsevers solution, and total hemolysis is measured spectrophotometrically. The results of the hemolysis assay can be performed with or without FH in order to understand the effect of the cofactor FH on total hemolysis. An exemplary protocol is provided herein. Briefly, 12 ml of rabbit red blood cells (RBC) is washed twice with GVB buffer (Gelatin Veronal buffer: Sigma, with 8 mM EGTA and 10 mM MgCl₂) and resuspended in 12 ml of ice cold GVB buffer. C3-deficient human serum is spiked with I μM of human C3, based on previous observations that 1 μM of C3 supports maximum hemolysis in this system. Three-fold eight-point serial dilutions of variants/fusion proteins in GVB buffer is done to achieve concentrations ranging from 260 μg/ml to 0.11 μg/ml in the reaction mixture. First, in a 96 well plate, 50 μl reaction mixture for each concentration point is prepared by adding 62.8% human serum, different concentrations of variants/fusion proteins with or without 200μg/mL FH. The hemolysis reaction is started by adding 50 μl of rabbit RBC and incubated in a microtiter plate at 37° C. for 30 minutes. All assays are done in triplicates and all dilutions are done in GVB buffer. For a maximum hemolysis control, de-ionized water is added to the RBC, and 0.154 M NaCl is added to the RBC for a no hemolysis control. After incubation, the plate is centrifuged at 2000 rpm for 5 minutes and 90 μl of supernatant is transferred to another 96 well plate. The percent hemolysis is quantitated by measuring optical density (OD) of lysed RBC at 412 nm.

The absorbances at 412 nm are converted to a percentage of hemolysis, utilizing maximum hemolysis from the control as 100% and the buffer control 0%.

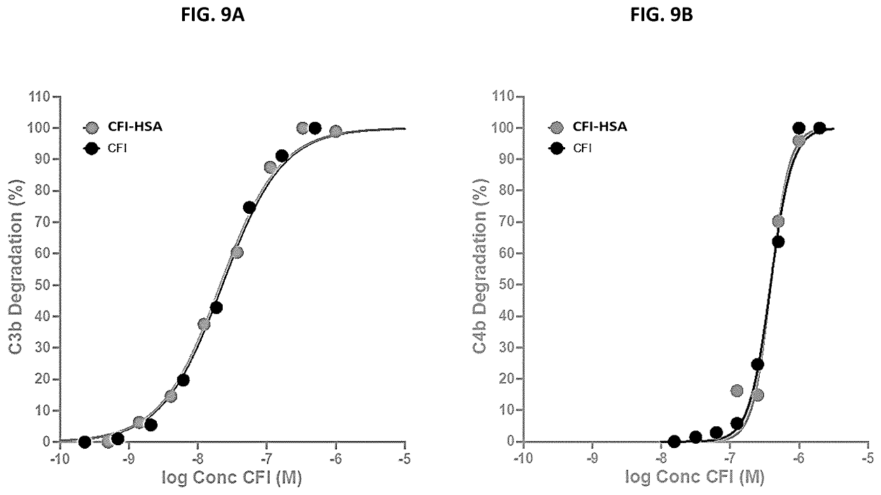

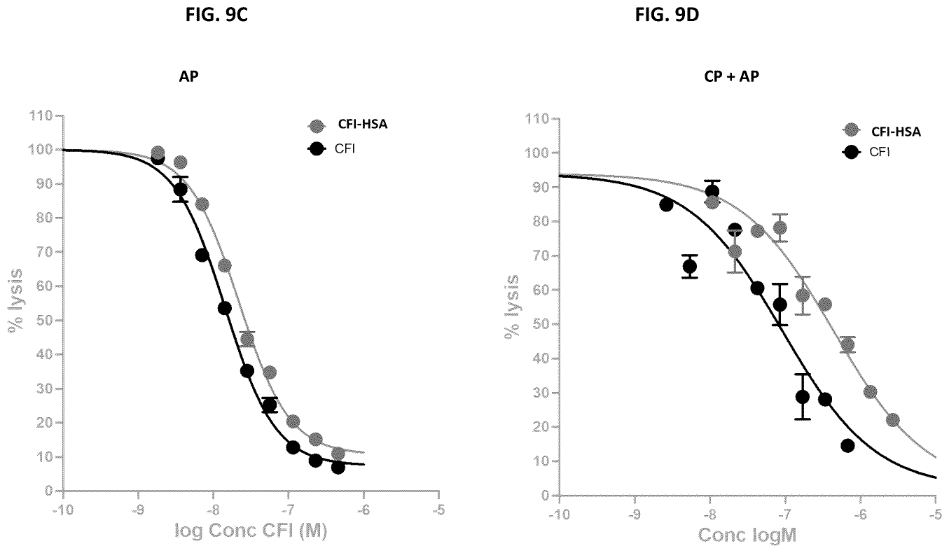

FIGS. 9A-9B depict dose response curves generated from the hemolysis assay for CFI with and without its cofactor Factor H, respectively. The dose response curves are generated by non-linear regression analysis and curve-fitting to a 4-parameter sigmoid curve in prism software. The data showed 50% alternative pathway activity (AP₅₀) of wild type CFI-HSA with FH, and plasma derived CFI with FH.

FIGS. 9C-9D depict dose response curves for percentage of hemolysis inhibition measured in the classical pathway and the alternative pathway, respectively, by plasma-derived CFI, and CFI-HSA wild type. These figures show that plasma-derived CFI and CFI-HSA wild type perform similarly in human serum.

These data showed that, at higher concentrations, both CFI-HSA and CFI-PD are active in the hemolysis assay. The inhibitory activity of CFI-HSA on the alternative pathway was similar to that of CFI-PD in the hemolysis assay. The hemolysis assay also showed that the inhibitory effect of CFI, both CFI-HSA and CFI-PD, on the alternative pathway increased significantly with cofactor FH.

The capacity to inhibit classical pathway hemolysis by variants/fusion proteins can be measured. An exemplary protocol is provided herein. Sheep red blood cells are activated by anti-SRBC antibodies (Amboceptor. Testline. UK). The SRBCs are suspended in gelatin veronal buffer (GVB). In the assay plates, a dilution series of the CFI variants are added followed by the activated SRBC and Factor B and I depleted serum at ˜1% (v/v). The activated SRBC are incubated with test articles for 30 mins. The cells are pelleted and the supernatant transferred to a separate plate for absorbance readings at 412 nM. Percentage lysis is calculated as follows: 100*(Absorbance test sample)/(Absorbance no CFI (0% inhibition)). Data is plotted and analyzed using four parameter non-linear regression (GraphPad Software. USA). IC₅₀ values are calculated for data from individual plates and averages are performed on logIC₅₀) values and transformed to concentration (nM) as summarized in Table 5.3.

The capacity to inhibit alternative pathway hemolysis by variants/fusion proteins can also measured. An exemplary protocol is provided herein. Sheep red blood cells are activated by anti-SRBC antibodies (Amboceptor. Testline. UK). The SRBCs are suspended in 8% (v/v) of normal human serum depleted of Factors B and H to which is added eculizumab to deposit C3b. The activated SRBC with deposited C3b are incubated with full-length Factor H (Complement Technologies. USA) and the test articles. After a 10 min incubation Factors B and D (Complement Technologies. USA) are added and incubated for a further 10 min. Finally, guinea pig serum (Sigma-Aldrich. UK) is added and incubated for 20 min. The cells are pelleted and the supernatant transferred to a separate plate for absorbance readings at 412 nM. Percentage lysis is calculated as follows: 100*(Absorbance test sample)/(Absorbance no CFI (0% inhibition)). Data is plotted and analyzed using four parameter non-linear regression (GraphPad Software. USA). IC₅₀ values are calculated for data from individual plates and averages are performed on logIC50 values and transformed to concentration (nM).

### Example 6: Pharmacokinetic Modeling to Determine Dosing in Humans Based on Non-Human Primate Data

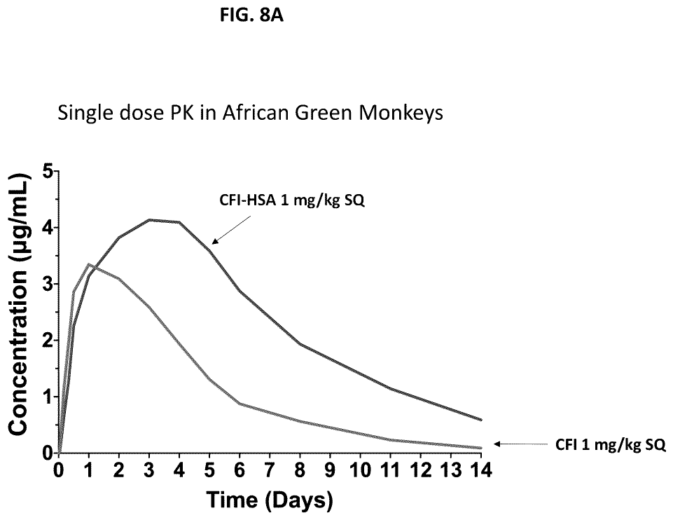

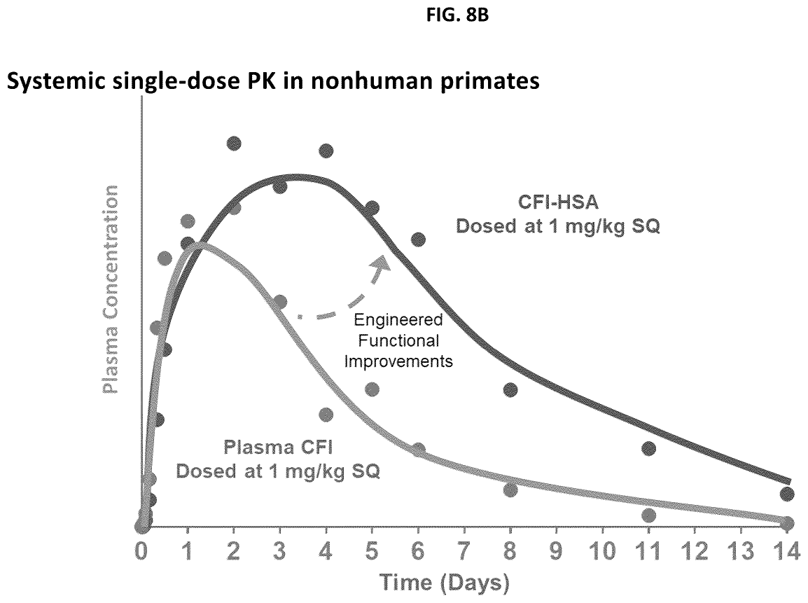

The variants/fusion proteins can be tested for concentration levels in plasma after a single subcutaneous dose in a model system, e.g. African green monkeys. The fusion construct comprised a human serum albumin (HSA) and a wild type CFI (CFI-HSA). FIG. 8A is a graph depicting the measured concentrations of the CFI-HSA fusion construct as compared to the free CFI, after subcutaneous administration to the monkeys at a dose of 1 mg/kg. The CFI-HSA fusion construct showed that it could achieve a target level of about 20 μg/ml. A measurable concentration of the CFI-HSA fusion construct persisted for up to 14 days, and the target concentrate of about 20 μg/ml was measured for about 7 days. These data support a weekly subcutaneous administration of a CFI-HSA fusion construct for therapeutic uses. These data support that weekly subcutaneous administration can be used for therapeutic purposes in humans for CFI-HSA-comparable modeling can be carried out for the variants and variant fusion constructs of the disclosure. FIG. 8B depicts the graph shown in FIG. 8A with the individual data points shown along the curves for additional clarity.

### Example 7: Characterization by C3b and C4b Cleavage Assays

### C3b Cleavage Reactions

For Example 7, reference to CFI-HSA refers to human serum albumin fused to the N-terminal end of wild type CFI (SEQ ID NO: 21).

In one exemplary protocol for the cleavage reactions using SDS-PAGE is as follows. First, for each CFI-HSA variant, the master reaction mixture was set up at room temperature containing the final concentrations of 500 nM of mini FH and 500 nM of C3b in HBS buffer (30 mM HEPES, 140 mM NaCl pH 7.4). The master reaction mixtures were transferred to 37° C. and allowed to equilibrate for 5 minutes. The cleavage reaction was initiated by the addition of CFI-HSA protein to a final concentration of 5 nM. A sample volume corresponding to 0.6 μg of C3b was withdrawn from the master mixtures for each time point measured and quenched by the addition of 5×SDS reducing sample buffer. Samples were run on a 9 or 10% SDS-PAGE gel and C3b cleavage was visualized by Coomassie staining. The amount of C3b cleavage that occurred was quantitated by densitometry. The C3b cleavage activity of wild type CFI-HSA was normalized as 100% in order to calculate the percentage of C3b cleavage activity of the CFI-HSA variants.

Another exemplary protocol for the cleavage reactions was performed as follows, as measured by ELISA. C3b cleavage reactions were performed using 1 nM CFI (variant or wild type), 500 nM cofactor mini FH, and 500 nM soluble human C3b incubated for 10 minutes at 37° C. in HEPES buffered saline (HBS). The reaction was quenched by the addition of 1 M NaCl in HBS. The reactions were further diluted to a final concentration of 5 nM C3b in buffer (HBS, 0.5M NaCl, 0.05% Tween 20) before proceeding with an iC3b ELISA. The C3b cleavage activity was determined from the amount of iC3b generated in the cleavage reaction. The amount of iC3b formed was assayed using the Micro Vue iC3b A006 ELISA kit (Quidel). The ELISA assay consists of a microplate coated with an iC3b specific monoclonal antibody for capture of formed iC3b during the reactions and detection of bound iC3b using an HRP-conjugated anti-iC3b antibody and a chromogenic substrate. The absorbance recorded is a relative measure of the iC3b product generated in the cleavage reactions. The fold difference of C3b cleavage activity of CFI variants relative to a reference molecule. CFI-HSA wild type, was calculated by dividing the background-corrected absorbance from CFI-HSA variants by the background-corrected absorbance for CFI-HSA wild type. Table 7.3 summarizes these results, presenting the fold difference of the median value for each CFI variant relative to the median value of the reference molecule. The fold differences were also calculated from SDS-PAGE gels. Samples from a C3b cleavage time course were run on a 9 or 10% SDS-PAGE gel and C3b cleavage was visualized by Coomassie staining. The amount of C3b cleavage that occurred was quantitated by densitometry and the data plotted and an apparent rate constant (k) for loss of band intensity determined by fit to an exponential decay. The fold difference of C3b cleavage activity of CFI variants relative to a reference molecule, the CFI-HSA wild type, was calculated by dividing the k-value from the CFI-HSA variants by the k-value for CFI-HSA wild type.

C3b cleavage by CFI variants was further characterized by determining the EC50 for the C3b cleavage. Briefly. C3b cleavage reactions were performed using 25 nM mini FH. 75 nM soluble human C3b and a dilution series of the CFI variants. Reaction mixtures at each of the concentrations of the CFI variants were incubated for 5 min at 37° C. in HBS. The reaction was quenched by the addition of 1 M NaCl in HBS. The reactions were further diluted to a final concentration of 5 nM C3b in buffer (HBS. 0.5M NaCl. 0.05% Tween 20) before proceeding with an iC3b ELISA. The amount of iC3b generated in the reaction was determined using the Micro Vue iC3b A006 ELISA kit (Quidel). The ELISA assay consists of a microplate coated with an iC3b specific monoclonal antibody for capture of formed iC3b during the reactions and detection of bound iC3b using an HRP-conjugated anti-iC3b antibody and a chromogenic substrate. The absorbance recorded is a relative measure of the iC3b product generated in the cleavage reactions. The EC50 values were calculated using a four-parameter non-linear regression fit without constraints in GraphPad Prism. Table 7.4 below summarizes the results of the iC3b ELISA titration analyses. EC50 values above 500 nM were set to be 500 nM. The cleavage reactions were also performed in the absence of mini FH where noted and analyzed in the same fashion as those containing mini FH.

### C4b Cleavage Reactions

CFI regulates the classical complement pathway by proteolytic inactivation of the C4b protein. CR1. a C3b/C4b receptor, and C4 binding protein (C4BP) act as cofactors for the CFI-catalyzed cleavage reaction of C4b. The C4b cleavage assay is a functional assay to determine the ability of CFI and variants thereof for C4b cleavage activity in the presence of either the CR1 or C4BP cofactors. Complement factor protein C2. which binds specifically to C4b and not to the CFI-cleaved product iC4b, was used for C4b capturing. The CFI-catalyzed cleavage of C4b was measured by measuring the decrease in the concentration of C4b bound to C2 protein, immobilized on an ELISA plate. The captured C4b protein was detected by Anti-C4c polyclonal rabbit Ab (DAKO. #A0065) in an ELISA assay. C4b cleavage activity by CFI-HSA was normalized as 100% to calculate the percentage of C4b cleavage activity of CFI variants.

For each CFI-HSA variant, the master reaction mixture was set up at room temperature containing the final concentrations of 250 nM cofactor (CR1 domains 1-3) and 250 nM human C4b in HBS buffer (30 mM HEPES, 140 mM NaCl pH 7.4). The master reaction mixtures were transferred to 37° C. and allowed to equilibrate for 5 minutes. The cleavage reaction was initiated by the addition of CFI-HSA protein to a final concentration of 250 nM. A sample volume corresponding to 0.6 μg of C3b was withdrawn from the master mixtures for each time point measured and quenched by the addition of 5×SDS reducing sample buffer followed by incubation at 95° C. for 5 minutes. Samples were run on a 9 or 10% SDS-PAGE gel and C4b cleavage was visualized by Coomassie staining. The amount of C4b cleavage that occurred was quantitated by densitometry. The C4b cleavage activity of wild type CFI-HSA was normalized as 100% in order to calculate the percentage of C4b cleavage activity of the CFI-HSA variants.

Another example of the C4b cleavage activity assay was performed as follows, to determine the C4b cleavage activity of CFI variants relative to a reference molecule, CFI-HSA wild type. The cleavage reaction was performed with 250 nM of the CFI variants in the presence of 250 nM of cofactor (CR1domains 1-3) and 250 nM human C4b, which was incubated for 30 minutes at 37° C. The reaction mixture was diluted 20-fold before addition to a blocked ELISA plate coated with a mouse monoclonal anti-C4c antibody. The absorbance recorded from the ELISA plate is a relative measure of the C4c product generated in the cleavage reactions and therefore a measure of C4b cleavage activity. The fold difference of C4b cleavage activity of CFI variants relative to a reference molecule, CFI-HSA wild type, was calculated by dividing the background-corrected absorbance from CFI-HSA variants by the background-corrected absorbance for CFI-HSA wild type. Table 7.3 below summarizes the fold differences of the C4b cleavage activity assay of CFI variants relative to the CFI-HSA reference molecule, as measured by C4c ELISA screen with CR1.

The EC₅₀ of the C4b cleavage by CFI variants was measured. The assay was performed using 250 nM cofactor (CR1 domains 1-3), 250 nM human C4b and a dilution series of the CFI variants. The reaction mixtures were incubated for 30 minutes at 37° C. and then the reaction mixture was diluted 20-fold before beginning the ELISA. The amount of generated C4c was measured by ELISA using a mouse monoclonal antibody specific towards C4c. The absorbance recorded from the ELISA plate is a relative measure of the C4c product generated in the cleavage reactions and therefore a measure of C4b cleavage activity. The EC50 values were calculated using a four-parameter non-linear regression fit without constraints in GraphPad Prism. EC50 values above 1000 nM were set to be 1000 nM The cleavage reactions were also performed in the absence of CR1 where noted and analyzed in the same fashion as those containing CR1. Table 7.4 summarizes the results of the C4c ELISA titration with the CR1 cofactor.

### Activity in the Absence of Cofactor

C4b cleavage reactions were carried out as described above in the absence of cofactor for a panel of CFI variants (Table 7.1). The results show that CFI variants with a C-terminal fusion protein that include a human CR1 domain maintained their ability to cleave C4b in the absence of cofactor in the reaction mixture. In contrast, the CFI variants lacking a CR1 C-terminal fusion did not maintain their ability to cleave C4b. These results suggest that CFI variants with a C-terminal CR1 fusion can be CR1 cofactor independent.

TABLE 7.1
CFI Variant Cleavage of C4b in the Absence of Cofactor

| | C4c no cofactor EC₅₀ | C4c with cofactor EC₅₀ |
|---|---|---|
| Const. No. | (nM) | (nM) |
| 66 | 88.63 | 69.1 |

C3b cleavage reactions were carried out as described above in the absence of cofactor for a panel of CFI variants (Table 7.2). The results show that CFI variants with a C-terminal fusion protein that include a human CR1 domain maintained their ability to cleave C3b in the absence of cofactor in the reaction mixture.

TABLE 7.2
CFI Variant Cleavage of C3b in the Absence of Cofactor

| | iC3b no cofactor EC₅₀ | iC3b with cofactor EC₅₀ |
|---|---|---|
| Const. No. | (nM) | (nM) |
| 66 | 2.92 | 3.2 |

### Single Point Screening of CFI Variants for C4b and C3b Cleavage

The fold difference of C3b cleavage activity of CFI variants relative to a reference molecule, CFI-HSA wild type, was calculated. The background-corrected absorbance from CFI-HSA variants was divided by the background-corrected absorbance for CFI-HSA wild type. Results are reported in Table 7.3.

TABLE 7.3
Variant Screening for C4b and C3b Cleavage

| Const. | iC3b ref | C4c ref |
|---|---|---|
| No. | fold | fold |
| 1 | 1.39 | 1.08 |
| 2 | 1.73 | 0.58 |
| 3 | 1.01 | 3.48 |
| 4 | 0.89 | 3.55 |
| 5 | 0.79 | 3.89 |
| 6 | 0.63 | 2.98 |
| 7 | 0.8 | 0.81 |
| 8 | 0.14 | 0.39 |
| 9 | 0.07 | 0.05 |
| 10 | 0.03 | 0.02 |
| 11 | −0.01 | −0.02 |
| 12 | 0.18 | 0.9 |
| 13 | 0.34 | 0.72 |
| 14 | 1.36 | 0.45 |
| 15 | 0.67 | 1.98 |
| 16 | 0.18 | 1.24 |
| 17 | 0.44 | 1.54 |
| 18 | 0.16 | 0.08 |
| 19 | 0.02 | −0.05 |
| 20 | 0 | 0.05 |
| 21 | 0.55 | 1.44 |
| 22 | 0.23 | 1.07 |
| 23 | 0.04 | 0.1 |
| 24 | 0.19 | 0.59 |
| 25 | 0.29 | 0.74 |
| 26 | 2.37 | 1.92 |
| 27 | 2.12 | 2.93 |
| 28 | 1.87 | 0.86 |
| 29 | 1.64 | 0.5 |
| 30 | 1.44 | 0.73 |
| 31 | 1.53 | 1.14 |
| 32 | 1.47 | 0.98 |
| 33 | 1 | 0.55 |
| 34 | 2.11 | 2.62 |
| 35 | 0.04 | 2.02 |
| 36 | 1.73 | 2.08 |
| 37 | 1.12 | 3.59 |
| 38 | 0.72 | 2.93 |
| 39 | 1.1 | 2.83 |
| 40 | 0.68 | 2.17 |
| 41 | 0.9 | 2.36 |
| 42 | 0.08 | 2.33 |
| 43 | 0.89 | 1.54 |
| 44 | 0.03 | 3.47 |
| 45 | 0.7 | 1.3 |
| 46 | 1.21 | 0.76 |
| 47 | 0.35 | 0.93 |
| 48 | 0 | 0.67 |
| 49 | 0.15 | 1.47 |
| 50 | 1.89 | 1.23 |
| 51 | 1.87 | 1.17 |
| 52 | 1.55 | 0.78 |
| 53 | 1.52 | 0.8 |
| 54 | 2.06 | 1.61 |
| 55 | 1.85 | 1.38 |
| 56 | 1.77 | 0.65 |
| 57 | 0.76 | 0.06 |
| 58 | 0.77 | 0.06 |
| 59 | 0.74 | 1.66 |
| 60 | 0.03 | 1.39 |
| 63 | 0.14 | 4.15 |
| 64 | 0.06 | 4.11 |
| 65 | 0.1 | 4.1 |
| 66 | 0.15 | 3.55 |
| 67 | −0.01 | 3.2 |
| 68 | 0.06 | 3.75 |
| 70 | 0.94 | 4.12 |
| 71 | 0.07 | 0.09 |
| 72 | 3.17 | 0.34 |
| 73 | 0.08 | −0.03 |
| 74 | 1.67 | 2.19 |
| 75 | −0.03 | 0.12 |
| 76 | 2.11 | 4.46 |
| 77 | 1.21 | 3.81 |
| 78 | 0 | −0.24 |
| 79 | 1.63 | 3.36 |
| 80 | 0.91 | 3.07 |
| 81 | 0.95 | 3.31 |
| 89 | 0.05 | 3.21 |
| 90 | 0.47 | 2 |
| 91 | 1.7 | 4.59 |
| 92 | 0.72 | 1.52 |
| 93 | 1.09 | 2.36 |
| 94 | | |
| 95 | 1 | 1 |
| 96 | | |

The specificity for C4b cleavage versus C3b cleavage and C3b cleavage versus C4b cleavage was determined. Specificity was calculated by normalizing EC50 values to CFI-HSA. For C4b cleavage the max value was set at 1000 nM and all values above that were set to 1000 nM. For C3b cleavage the max value was set at 500 nM and all values above that were set to 500 nM. Results are reported in Table 7.4.

TABLE 7.4
EC₅₀ Values for Variants in C4b and C3b Cleavage Assays

| Const. | iC3b EC₅₀ | C4c EC₅₀ |
|---|---|---|
| No. | (nM) | (nM) |
| 1 | | 183.29 |
| 2 | 28.64 | 243.81 |
| 3 | 18.92 | 102.86 |
| 4 | 21.43 | 186.45 |
| 5 | 24.75 | 183.19 |
| 6 | 34.66 | 104.28 |
| 7 | 15.76 | 908.5 |
| 8 | | |
| 9 | | |
| 10 | | |
| 11 | | |
| 12 | | |
| 13 | | |
| 14 | 40.89 | |
| 15 | 35.14 | 488.76 |
| 16 | 85.61 | |
| 17 | | |
| 18 | | |
| 19 | | |
| 20 | | |
| 21 | 30.52 | 240.39 |
| 22 | 101.84 | 1450 |
| 23 | 155.92 | 1207 |
| 24 | | |
| 25 | | |
| 26 | 6.39 | 99.62 |
| 27 | 6.26 | 47.02 |
| 28 | 11.82 | 90.43 |
| 29 | 10.63 | 210.39 |
| 30 | | |
| 31 | | |
| 32 | | |
| 33 | 16.36 | 707 |
| 34 | 8.7 | 93.16 |
| 35 | 347.26 | 744.91 |
| 36 | 13.05 | 205.41 |
| 37 | 20.33 | |
| 38 | 39.83 | 218.94 |
| 39 | 31.43 | 203 |
| 40 | 43.15 | 214.66 |
| 41 | 31.37 | 223.89 |
| 42 | 168.11 | 379.44 |
| 43 | | |
| 44 | | |
| 45 | 61.06 | |
| 46 | | |
| 47 | 154.42 | |
| 48 | 1000 | 811.69 |
| 49 | 89.23 | |
| 50 | 12.8 | |
| 51 | 12.48 | |
| 52 | 22.54 | |
| 53 | 20.74 | |
| 54 | 7.38 | |
| 55 | 12.38 | |
| 56 | 9.67 | |
| 57 | 17.47 | |
| 58 | 8.15 | |
| 59 | 24.9 | |
| 60 | 155.78 | 366.86 |
| 63 | | |
| 64 | | |
| 65 | | |
| 66 | 3.17 | 69.11 |
| 67 | | |
| 68 | | |
| 70 | 17.48 | 16.65 |
| 71 | 32.7 | 679 |
| 72 | 3.12 | |
| 73 | 54.61 | 1606 |
| 74 | 5.79 | 47.13 |
| 75 | | |
| 76 | | |
| 77 | | |
| 78 | | |
| 79 | | |
| 80 | | |
| 81 | | |
| 89 | | |
| 90 | 2.37 | 20.78 |
| 91 | 6.69 | 13.82 |
| 92 | 46.29 | |
| 93 | | |
| 94 | 50.1 | 412.1 |
| 95 | 28.6 | 292.4 |
| 96 | 62.4 | 339.6 |

### Example 8: Tunability and Selection of CFI Variants for C3b, C4b, or both C3b and C4b

For Example 8, reference to CFI-HSA refers to human serum albumin fused to the N-terminal end of wild type CFI (SEQ ID NO: 21).

For the assays where EC50 values were determined, the specificity for C4b cleavage versus C3b cleavage and C3b cleavage versus C4b cleavage were calculated by normalizing to CFI-HSA, as shown in Table 7.4. Specificity is shown in Table 8.1. Specificity for C4b was calculated as follows: (C4b EC50 CFI-HSA/C4b EC50 variant)/(C3b EC50 CFI-HSA/C3b EC50 variant). Specificity for C3b was calculated as follows: (C3b EC50 CFI-HSA/C3b EC50 variant)/(C4b EC50 CFI-HSA/C4b EC50 variant). Results are reported in Table 8.1.

TABLE 8.1
C3b and C4b Cleavage Specificity

| Const. | Specifity | Specifity |
|---|---|---|
| No. | C3b | C4b |
| 1 | | |
| 2 | 1.31 | 0.77 |
| 3 | 0.53 | 1.88 |
| 4 | 0.85 | 1.18 |
| 5 | 0.72 | 1.38 |
| 6 | 0.29 | 3.4 |
| 7 | 5.63 | 0.18 |
| 8 | | |
| 9 | | |
| 10 | | |
| 11 | | |
| 12 | | |
| 13 | | |
| 14 | | |
| 15 | 1.36 | 0.74 |
| 16 | | |
| 17 | | |
| 18 | | |
| 19 | | |
| 20 | | |
| 21 | 0.77 | 1.3 |
| 22 | 1.39 | 0.72 |
| 23 | 0.76 | 1.32 |
| 24 | | |
| 25 | | |
| 26 | 1.52 | 0.66 |
| 27 | 0.73 | 1.36 |
| 28 | 0.75 | 1.34 |
| 29 | 1.93 | 0.52 |
| 30 | | |
| 31 | | |
| 32 | | |
| 33 | 4.22 | 0.24 |
| 34 | 1.05 | 0.96 |
| 35 | 0.21 | 4.77 |
| 36 | 1.54 | 0.65 |
| 37 | | |
| 38 | 0.54 | 1.86 |
| 39 | 0.63 | 1.58 |
| 40 | 0.49 | 2.06 |
| 41 | 0.7 | 1.43 |
| 42 | 0.22 | 4.53 |
| 43 | | |
| 44 | | |
| 45 | | |
| 46 | | |
| 47 | | |
| 48 | 0.05 | 18.28 |
| 49 | | |
| 50 | | |
| 51 | | |
| 52 | | |
| 53 | | |
| 54 | | |
| 55 | | |
| 56 | | |
| 57 | | |
| 58 | | |
| 59 | | |
| 60 | 0.23 | 4.35 |
| 63 | | |
| 64 | | |
| 65 | | |
| 66 | 2.13 | 0.47 |
| 67 | | |
| 68 | | |
| 70 | 0.09 | 10.74 |
| 71 | 2.03 | 0.49 |
| 72 | | |
| 73 | 2.87 | 0.35 |
| 74 | 0.8 | 1.26 |
| 75 | | |
| 76 | | |
| 77 | | |
| 78 | | |
| 79 | | |
| 80 | | |
| 81 | | |
| 89 | | |
| 90 | 1.35 | 0.74 |
| 91 | 0.32 | 3.15 |
| 92 | | |
| 93 | | |
| 94 | 1.29 | 0.77 |
| 95 | 1 | 1 |
| 96 | 0.86 | 1.17 |

### Example 10: Intravitreal Injections

For Example 10, reference to CFI-HSA refers to human serum albumin fused to the N-terminal end of wild type CFI (SEQ ID NO: 21).

### Pharmacokinetics of CFI-HSA Following Intravitreal Injection

The ocular pharmacokinetics of the N-terminal albumin fusion of wild-type CFI-HSA were examined after intravitreal dosing to six African Green Monkeys (AGMs). The six animals were divided into two groups treated at 2 dose levels: one group received a single intravitreal injection of 500 μg of CFI-HSA (right eye, OD, N=3) and the other group received a single intravitreal injection of 250 μg of CFI-HSA (right eye, OD, N=3). The left eye (OS) of all six animals was injected with an equivalent volume of 100 μL of sterile PBS for injection as a vehicle control. Non-terminal, vitreous humor samples (100 μL) were taken on days 1, 7, 14, 21 and 28 post dosing. Vitreous humor CFI-HSA drug concentrations were determined using a quantitative electrochemiluminescence (ECL) antigen assay optimized for measuring CFI-HSA in vitreous humor of AGMs. The assay employs coating of anti-CFI antibody (clone OX21, LS Bio, Seattle WA) at 2 μg/ml on the Meso Scale Discovery (MSD, Rockville, MA) assay plate to capture the CFI-HSA levels. Detection of the captured CFI-HSA is performed with a goat polyclonal anti-HSA antibody (Abcam, Cambridge, MA) at 0.5 μg/ml conjugated with SULFO-TAG which emits light [electrochemiluminescence (ECL)] on application of an electric potential. The ECL relative light units (RLU) is measured on a MESOR SECTOR S 600 Reader and the unknown CFI-HSA concentrations in vitreous humor are interpolated from a standard curve ranging from 0.05 μg/ml to 40 μg/ml Factor I-HSA.

Non-compartmental analysis yielded apparent ocular terminal half-lives of 3.6 and 4.1 days for the 250 and 500 μg dose levels, respectively.

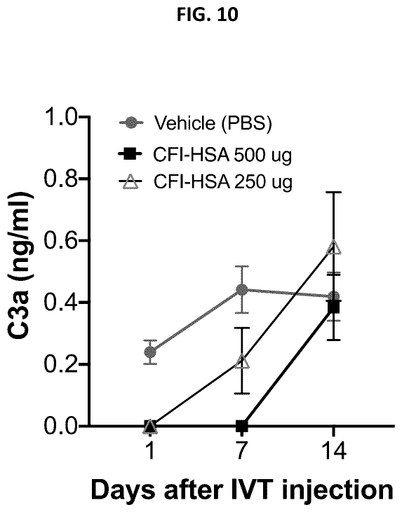

Complement Component 3a (C3a) levels in vitreous humor were determined by ELISA using the Quidel kit for C3a ELISA (FIG. 10). CFI-HSA fusion protein reduced ocular C3a levels in a dose-dependent manner up to 7 days after intraocular injection. The increase in C3b degradation by CFI-HSA reduces the complex formation between C3b and Bb which leads to a reduction of C3 cleavage into C3a and C3b via the amplification loop of the alternative pathway. Other variants and fusion constructs can be tested in this same model for their activity.

### Example 11: CFI-HSA and Plasma CFI Pharmacokinetics in Mouse Plasma

For Example 11, reference to CFI-HSA refers to human serum albumin fused to the N-terminal end of wild type CFI (SEQ ID NO: 21).

The pharmacokinetics of the N-terminal albumin fusion of wild-type CFI (CFI-HSA) is examined after intravenous and subcutaneous administration to CD-1 mice. Employing a sparse sampling design with up to two samples per mouse and three mice sampled at each timepoint, CD-1 mice are divided into four groups and treated with a single dose of either the plasma purified wild type CFI, or the recombinant wild-type CFI-HSA.

To compare the circulating half-life in plasma and bioavailability of the plasma-derived CFI and CFI-HSA, animals are dosed with either plasma-derived CFI or CFI-HSA both intravenously and subcutaneously. Plasma-derived CFI is administered at 1.3 mg/kg intravenously (group 1) and 6.5 mg/kg subcutaneously (group 2). CFI-HSA is administered at 3 mg/kg intravenously (group 3) and subcutaneously (group 4). An additional 3 animals receive a single dose of an equivalent volume of PBS delivered subcutaneously as a vehicle control (group 5; not shown). Blood (˜30-50 μL) is collected in EDTA at various time points from 5 minutes to 144 hours post dosing and plasma separated by centrifugation.

CFI-HSA and plasma CFI concentrations are determined with a quantitative electrochemiluminescence (ECL) antigen assay for CFI-HSA and plasma CFI in CD-1 mouse EDTA plasma. For the CFI assay, the mouse monoclonal anti-Factor I antibody (MAB12907, Abnova, Taipei City, Taiwan) is coated at 2 μg/ml on the Meso Scale Discovery (MSD, Rockville, MA) assay plate to capture the plasma CFI. Detection of the captured CFI is performed with a mouse monoclonal anti-CFI antibody (clone 3R/8, CABT-47940MH, Creative diagnostic, Shirley NY) at 0.5 μg/ml conjugated with SULFO-TAG which emits light [electrochemiluminescence (ECL)] on application of an electric potential. For the CFI-HSA assay, the mouse monoclonal anti-CFI antibody (clone 3R/8, CABT-47940MH) is coated at 1 μg/ml on the Meso Scale Discovery (MSD, Rockville, MA) assay plate to capture the CFI-HSA. Detection of the captured CFI-HSA is performed with a rabbit polyclonal anti-HSA antibody (ab24207, Abcam, Cambridge, MA) at 2 μg/ml conjugated with SULFO-TAG which emits light [electrochemiluminescence (ECL)] on application of an electric potential. The ECL relative light units (RLU) is measured on a MESOR SECTOR S 600 Reader and unknown plasma CFI and CFI-HSA concentrations are interpolated from the standard curves.

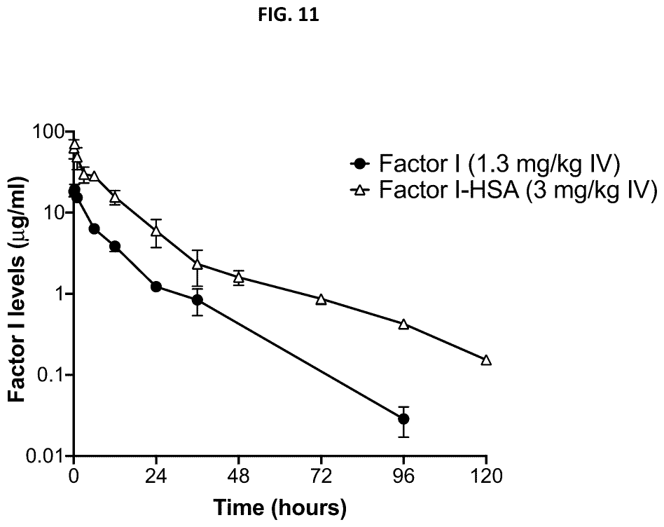

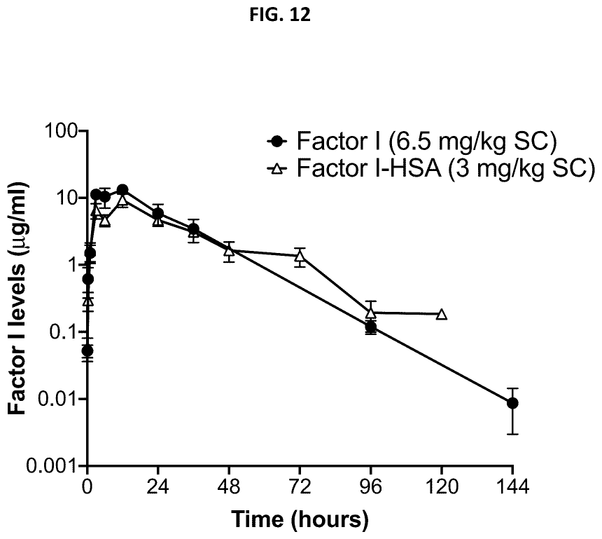

The circulation half-life following intravenous infusion of CFI-HSA is longer (˜22 hours) than the non-fusion version plasma CFI protein (˜13 hours), indicating that fusion of HSA to the CFI protein increases the half-life of compared to unfused CFI. Importantly, the bioavailability of CFI (53.6%) is similar to the CFI-HSA (46.7%), indicating that fusion of HSA to the CFI protein did not adversely affect bioavailability of CFI after subcutaneous dosing. Fusing HSA to CFI protein increases half-life by ˜2-fold compared to the non-fusion version CFI protein after intravenous (FIG. 11) or subcutaneous dosing (FIG. 12). Similar exposure is achieved with a 2-fold lower dose of fusion protein CFI-HSA compared to CFI after subcutaneous injection (FIG. 12).

The circulation of other variants and fusion proteins can be tested in a similar fashion.

### Example 12: CFI-HSA Bioactivity Following Intravenous Administration in Rodent Models of Complement Activation

A rat model of peripheral nerve injury was developed to study complement involvement in Wallerian degeneration due to mechanical damage of the myelinated sciatic nerve. Male CD Sprague Dawley rats (Charles River Laboratories) weighing between 300 and 350 g at enrollment were anesthetized with a mixture of 2 to 2.5% isoflurane USP (Abbot Laboratories, Montreal, Canada) in oxygen, and placed on a heating pad to maintain body temperature. Both legs underwent a sterile surgery to expose the sciatic nerve. One leg underwent a sciatic nerve injury (SNI) by clamping the sciatic nerve three times for 10 seconds using Dumont #7 forceps. The contralateral leg received no clamp injury and served as an internal control for each subject.

Immediately following induction of SNI, animals can receive an intravenous injection of a variant or fusion protein of the disclosure. A subcutaneous injection of slow-release buprenorphine (0.01 mg/kg) is also administered for pain management. 4 or 24 hours after SNI, 5 animals from each treatment group are sacrificed by exsanguination.

At sacrifice, a 1 cm (0.5 cm proximal and distal to the site of injury) piece of nerve is collected from the injured (ipsilateral) and sham legs, snap frozen, and stored at-80° C. until processed for mass spectrometry analysis (Phenoswitch Bioscience, Canada). K2-EDTA plasma samples are collected prior to SNI (baseline) as well as 1, 4, and 24 hours (where applicable) after SNI for evaluation of complement component fragments by mass spectrometry (MS). Cytokine and chemokine levels (Rat 27 plex Multiplex Immunoassay analyzed with a BioPlex 200 Cytokine Array, Assay Kit Millipore MILLIPLEX, performed by Eve Technologies, Calgary, Canada) are assessed in K2-EDTA plasma collected at baseline (vehicle only), 4, and 24 hours (where applicable) after SNI. At sacrifice, whole blood and serum are collected for clinical pathology evaluation [complete blood counts (CBC) and serum chemistry; Biovet Inc., Canada].

Mass Spectrometry Analysis of In vivo Samples

Samples are denatured and precipitated, with a wash and buffer exchange before N-terminal labeling via reductive amine dimethylation. Samples are then digested with trypsin (or a mix of trypsin and chymotrypsin) before analysis via LC-MS/MS using SWATH. SWATH data is integrated on an ion library produced for each species and sample type. Top 10 peptides per protein contained in the ion library are integrated, and a peptide centric analysis is carried out for specific quantification of C3, C5, C4 and CFB N-terminal labeled peptides.

Cleavage products resulting from catalytic activity on C3b are monitored in the nerve tissue (membrane-bound fragments) (FIG. 21A) and circulation (soluble fragments in plasma) (FIG. 21B) by mass spectrometry. CFI-HSA (Y408L; N531G variant) cleavage activity results in 2 major cleavage fragments detected by mass spectrometry: C3dg and C3f. Cleavage of surface-bound C3b by CFI will result in a surface-bound C3dg fragment and a soluble C3f fragment. Both fragments are soluble when formed from soluble C3b. Therefore, the detection of C3dg in plasma may be attributed to cleavage of soluble C3b while detection of C3dg in tissue may result from cleavage of membrane-bound C3b. N-terminal labelled C3dg (E [2Me]DVPAADLSDQVPDTDSETR) (SEQ ID NO: 24) is the product of CFI cleavage of iC3b. The activity of CFI variants is determined as the percent of C3dg peptides with N-terminal labeling (termed “activated C3dg”) multiplied by the total signal size of C3dg (EDVPAADLSDQVPDTDSETR) (SEQ ID NO: 48).

### Example 13: In Vivo Activity in Peripheral Nerve Injury Rat Model

The efficacy of a panel of CFI variants on complement activation in a sciatic nerve (SN) injury (SNI) rat model can be determined. Immediately following induction of SNI, animals receive an IV injection with a variant or fusion protein or control (1×PBS) at a dose volume of 5 mL/kg. 24 hours after SNI, all animals are sacrificed by exsanguination. Cytokine and chemokine levels (Rat 27 plex Multiplex Immunoassay analyzed with a BioPlex 200 Cytokine Array. Assay Kit Millipore MILLIPLEX. performed by Eve Technologies. Calgary. Canada) are assessed in K2-EDTA plasma collected at 4 and 24 hours after SNI. At sacrifice, serum is collected for serum chemistry [Biovet Inc., Canada].

The activity of variants and fusion proteins is monitored by detecting CFI cleavage products (C3dg and C3f) using mass spectrometry. N-terminal labelled C3f (S [2Me]EETK[2Me]QNEGF) (SEQ ID NO: 48) is the product of CFI cleavage of C3b and N-terminal labelled C3dg (E[2Me]DVPAADLSDQVPDTDSETR) (SEQ ID NO: 24) is the product of CFI cleavage of iC3b. Total activated C3f is determined as the percent of C3f peptides with N-terminal labeling (S[2Mc]EETK[2Me]QNEGF) (SEQ ID NO: 48) multiplied by the total peptide signal size of C3f (SEETKQNEGF) (SEQ ID NO: 49).

### Example 14: In Vivo Activity of CFI Variants in Cecal Ligation and Puncture Model

CFI variant and fusion protein effect on limiting complement activation in a model of cecal ligation and puncture (CLP)-induced sepsis in rats can be assessed.

A rat model of non-aseptic sepsis can be used to study complement involvement following a cecal ligation and puncture (CLP) surgery. This surgery provides three facets of complement activation and inflammation (mechanical damage, bacterial exposure, and ischemic injury) that make it particularly relevant as a screening tool for other indications. Male CD Sprague Dawley rats (Charles River Laboratories) weighing between 300 and 350 g at enrollment are anesthetized with a mixture of 2 to 2.5% isoflurane USP (Abbot Laboratories. Montreal, Canada) in oxygen, and placed on a heating pad to maintain body temperature. Sepsis is induced by a CLP surgical procedure. A midline incision is made in the abdominal wall, the cecum exteriorized, and ligated with a nylon suture (4-0) proximal to the ileo-cecal valve, then perforated using a 16-gauge needle passed through the distal portion of the cecum resulting in a small amount of cecum contents entering the abdominal cavity. The abdominal wall and skin are then sutured.

Immediately following the CLP procedure, animals receive an intravenous injection of selected variants or fusion proteins or control article (1×PBS) at a dose volume of 5 mL/kg. 16 hours after CLP surgery all animals are sacrificed by exsanguination. K2-EDTA plasma samples are collected the day prior to enrolment (baseline). 3. and 16 hours after CLP for evaluation of complement component fragments by mass spectrometry (MS) and cytokine/chemokine levels (Rat 27 plex Multiplex Immunoassay analyzed with a BioPlex 200 Cytokine Array. Assay Kit Millipore MILLIPLEX. performed by Eve Technologies. Calgary. Canada). Whole blood and serum are collected for clinical pathology evaluation [complete blood counts (CBC) and serum chemistry; Biovet Inc., Canada] at baseline and 16 hours.

### Example 15: In Vivo Activity of CFI Variants in Acute Respiratory Distress Syndrome

Evaluation of the therapeutic effects of CFI variants and fusion proteins in an LPS-induced acute respiratory distress syndrome (ARDS) mouse model can be studied.

Purpose: The purpose of this study is to assess the efficacy of variants and fusion proteins to limit complement mediated acute pulmonary inflammation in a mouse model of ARDS induced by a single administration of lipopolysaccharide (LPS).

A mouse model of aseptic ARDS can be used used to study complement involvement following an intratracheal instillation (IT) of LPS. Male C57BL/6 mice (Charles River Laboratories) weighing 20 to 25 g at enrolment are anesthetized under isoflurane and intratracheally instilled with 50 μg LPS (1 mg/mL LPS isolated from E. coli 0111:B4 in 0.9% saline solution, Sigma).

Three hours following the CLP procedure, animals receive an intravenous injection of 5 variants or fusion proteins or or control article (1×PBS) at a dosing volume of 5 mL/kg. To evaluate the potential impacts of repeat daily dosing. 27 hours post-LPS IT, animals can receive a second 5 mg/kg dose. A sham arm is subjected to a 50 μL intratracheal instillation of 0.9% saline solution (n=5) without any IV treatment.

K₂-EDTA plasma, lung tissue, and bronchoalveolar lavage fluid (BALF) samples are collected at sacrifice for evaluation of complement component fragments by mass spectrometry (MS). BALF is harvested in three 300 μL perfusions of the right lung with cold PBS 1× containing Protease Inhibitor 1× (SigmaFAST R). Cytokine and chemokine levels (Mouse 31 plex Multiplex Immunoassay analyzed with a BioPlex 200 Cytokine Array, Assay Kit Millipore MILLIPLEX, performed by Eve Technologies, Calgary, Canada) are assessed in K2-EDTA plasma, BALF, and lung tissue (homogenized in PBS 1×+0.1% Triton X-100 with protease cocktail inhibitors) collected at sacrifice. At sacrifice, whole blood and serum are collected for clinical pathology evaluation [complete blood counts (CBC) and serum chemistry; Biovet Inc., Canada]. A cell count differential is performed on BALF samples to assess leukocyte recruitment to the lung.

LPS is a known alternative complement pathway inducing agent. CFI variant or fusion activity on circulating C3b cleavage products can be assessed using mass spectrometry. Percent activated C3f is determined as the percent of the C3f peptide with N-terminal labeling (S[2Me]EETK[2Me]QNEGF) (SEQ ID NO: 48) multiplied by the total peptide signal size of C3f (SEETKQNEGF) (SEQ ID NO: 49).

1. A complement factor I (CFI) variant comprising at least one modification with respect to a wild type CFI, wherein the CFI variant is capable of modulating the complement system, wherein the CFI variant has at least one improved characteristic as compared to the wild type CFI, and wherein the variant is selected from those presented in Table 2 or Table 3.

2. The CFI variant of claim 1, wherein the improved characteristic is selected from an increase in half-life or bioavailability, or increase or decrease in any one or more of activity, substrate specificity, potency, substrate affinity, cofactor affinity and catalytic capability.

3. (canceled)

4. The CFI variant of claim 2, wherein the improved characteristic is an increase in activity, and wherein the increase in activity comprises:
(a) an increase in the cleavage of C3b and/or C4b, as compared to wild type CFI;
(b) an increase in the cleavage of C3b as compared to the wild type CFI, and does not comprise an increase in the cleavage of C4b;
(c) an increase in the cleavage of C4b as compared to the wild type CFI, and does not comprise an increase in the cleavage of C3b;
(d) an increase in the generation of iC3b;
(e) an increase in the generation of C3dg and/or C3c from iC3b;
(f) a reduction in the levels of C3b α-chain;
(g) an increase in the proteolysis of a peptide substrate;
(h) a reduction in the levels or function of membrane attack complex (MAC);
(i) a reduction of an amplification of the complement system; or
(j) a combination of (a)-(i).

5-15. (canceled)

16. The CFI variant of claim 2, wherein the improved characteristic is a decrease in activity for C3b and/or C4b.

17. (canceled)

18. The CFI variant of claim 2, wherein the improved characteristic is an increase in specificity for a substrate, and wherein the increase in specificity comprises:
(a) an increase in the specificity for C3b or C4b, as compared to wild type CFI;
(b) an increase in the specificity for C3b and/or C4b, as compared to wild type CFI;
(c) an increase in the specificity for C3b, as compared to wild type CFI; or
(d) an increase in the specificity for C4b, as compared to wild type CFI.

19-27. (canceled)

28. The CFI variant of claim 1, wherein the CFI variant comprises at least one modification corresponding to a wild type CFI having the amino acid sequence set forth in SEQ ID NO: 1 or SEQ ID NO: 5.

29. The CFI variant of claim 1, wherein the CFI variant is a chimera comprising one or more domains from a human CFI, and wherein the human CFI further comprises a substitution of one or more amino acid residues for amino acid residues of a corresponding region from a non-human species CFI.

30. (canceled)

31. The CFI variant of claim 1, wherein the CFI variant is a chimera, and wherein the modification comprises the substitution of one or more amino acid residues of the CFI with amino acid residues from a corresponding region of a non-CFI serine protease.

32. (canceled)

33. The CFI variant of claim 1, wherein the CFI variant comprises:
(a) an A chain and a B chain, wherein the CFI variant comprises one or more modifications at the interface of the A chain and the B chain;
(b) one or more modifications at a C-terminal region of the CFI variant; or
(c) one or more modifications at one or more N-linked glycosylation sites of the CFI variant;
(d) one or more modifications in the SPD domain of the CFI, optionally comprising one or more modifications at any one or more of the autolysis loop, the 99 loop, the S1 pocket entrance, or the activation loop of SPD, or any one or more of the domains presented in FIG. 1;
(e) a substitution of an autolysis loop of the CFI (REKDNERVES, SEQ ID NO: 9) for an autolysis loop of trypsin (NTASSGADYPDE, SEQ ID NO: 10), wherein the autolysis loop occurs between positions corresponding to position 456 and position 465 in a CFI having the amino acid sequence set forth in SEQ ID NO: 5;
(f) one or more modifications at an active site of the CFI;
(g) an A chain and a B chain, wherein the CFI variant comprises a structural arrangement from N-terminus to C-terminus as (A chain)-(optional linker)-(B chain);
(h) an A chain and a B chain, wherein the CFI variant comprises a structural arrangement from N-terminus to C-terminus as (B chain)-(optional linker)-(A chain); or
(i) each one of the SPD, the FIMAC domain, the SRCR domain, the LDLr1 domain, and the LDLr2 domain, and any other domains presented in FIG. 1.

34-43. (canceled)

44. The CFI variant of claim 1, wherein the CFI variant does not comprise all of the SPD, the FIMAC domain, the SRCR domain, the LDLr1 domain, and the LDLr2 domain.

45. The CFI variant of claim 44, wherein the CFI variant comprises the SPD, optionally wherein the CFI variant comprises the amino acid sequence set forth in SEQ ID NO: 12.

46. (canceled)

47. The CFI variant of claim 1, wherein the CFI variant is sialylated.

48. The CFI variant of claim 1, wherein the CFI variant is active, optionally wherein the CFI variant is activated by furin or a variant thereof.

49-53. (canceled)

54. The CFI variant of claim 1, wherein the CFI variant is a first component of a fusion construct comprising a first component and at least a second component, and the CFI variant is fused to the second component.

55-58. (canceled)

59. The CFI variant of claim 54, wherein the second component is a half-life extender selected from albumin, PEG, a non-biodegradable polymer, a biodegradable polymer, and Fc.

60-77. (canceled)

78. The CFI variant of claim 54, wherein the fusion construct further comprises a third component.

79-82. (canceled)

83. A fusion construct comprising at least a first, second, and third component, wherein the first component comprises a wild type CFI or variant thereof (CFI variant), wherein the second component comprises a FH or CR1, and the third component comprises a FH or CR1.

84-104. (canceled)

105. A fusion construct comprising any of the fusion constructs of Table 3.

106. A pharmaceutical composition comprising the CFI variant of claim 1, and optionally a pharmaceutically acceptable excipient.

107. A method of modulating the complement system, comprising contacting a sample in vitro or contacting a tissue in vivo with the CFI variant of claim 1.

108-114. (canceled)

115. A method of treating a non-ocular condition in a subject in need thereof, the method comprising administering to the subject a therapeutically effective amount of the CFI variant of claim 1.

116-129. (canceled)

130. A method of treating an ocular condition in a subject in need thereof, the method comprising administering to the subject a therapeutically effective amount of the CFI variant of claim 1.

131-134. (canceled)

135. A cell comprising one or more nucleic acids encoding the CFI variant of claim 1, and comprising one or more nucleic acids encoding furin.

136. A method of generating the CFI variant of 1, in an activated state, the method comprising producing the CFI variant recombinantly in a cell comprising one or more nucleic acids encoding the CFI variant, and comprising one or more nucleic acids encoding furin.


SEQUENCE LISTING (51 sequences)

SEQ ID NO: 1 - protein, 583 aa, Homo sapiens
MKLLHVFLLFLCFHLRFCKVTYTSQEDLVEKKCLAKKYTHLSCDKVFCQPWQRCIEGTCVCKLPYQCPKNGTAVCATNRRSFPTYCQQKSLECLHPGTKFLNNGTCTAEGKFSVSLKHGNTDSEGIVEVKLVDQDKTMFICKSSWSMREANVACLDLGFQQGADTQRRFKLSDLSINSTECLHVHCRGLETSLAECTFTKRRTMGYQDFADVVCYTQKADSPMDDFFQCVNGKYISQMKACDGINDCGDQSDELCCKACQGKGFHCKSGVCIPSQYQCNGEVDCITGEDEVGCAGFASVTQEETEILTADMDAERRRIKSLLPKLSCGVKNRMHIRRKRIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 2 - protein, 20 aa, Mus musculus
METDTLLLWVLLLWVPGSTG

SEQ ID NO: 3 - protein, 18 aa, Homo sapiens
MKLLHVFLLFLCFHLRFC

SEQ ID NO: 4 - protein, 1231 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 5 - protein, 565 aa, Homo sapiens
KVTYTSQEDLVEKKCLAKKYTHLSCDKVFCQPWQRCIEGTCVCKLPYQCPKNGTAVCATNRRSFPTYCQQKSLECLHPGTKFLNNGTCTAEGKFSVSLKHGNTDSEGIVEVKLVDQDKTMFICKSSWSMREANVACLDLGFQQGADTQRRFKLSDLSINSTECLHVHCRGLETSLAECTFTKRRTMGYQDFADVVCYTQKADSPMDDFFQCVNGKYISQMKACDGINDCGDQSDELCCKACQGKGFHCKSGVCIPSQYQCNGEVDCITGEDEVGCAGFASVTQEETEILTADMDAERRRIKSLLPKLSCGVKNRMHIRRKRIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 6 - protein, 6 aa, synthetic construct
GGSSGG

SEQ ID NO: 7 - protein, 585 aa, Homo sapiens [sequence omitted]

SEQ ID NO: 8 - protein, 415 aa, Homo sapiens
MRLLAKIICLMLWAICVAEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEAGGGGGGGGGGGGGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRENLYFQGHHHHHH

SEQ ID NO: 9 - protein, 10 aa, Homo sapiens
REKDNERVFS

SEQ ID NO: 10 - protein, 12 aa, unidentified
NTASSGADYPDE

SEQ ID NO: 11 - protein, 10 aa, Mus musculus
RGKDNQKVYS

SEQ ID NO: 12 - protein, 260 aa, synthetic construct
KLSCGVKNRMHIRRKRIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 13-15 - skipped (3 entries)

SEQ ID NO: 16 - protein, 1135 aa, synthetic construct
KVTYTSQEDLVEKKCLAKKYTHLSCDKVFCQPWQRCIEGTCVCKLPYQCPKNGTAVCATNRRSFPTYCQQKSLECLHPGTKFLNNGTCTAEGKFSVSLKHGNTDSEGIVEVKLVDQDKTMFICKSSWSMREANVACLDLGFQQGADTQRRFKLSDLSINSTECLHVHCRGLETSLAECTFTKRRTMGYQDFADVVCYTQKADSPMDDFFQCVNGKYISQMKACDGINDCGDQSDELCCKACQGKGFHCKSGVCIPSQYQCNGEVDCITGEDEVGCAGFASVTQEETEILTADMDAERRRIKSLLPKLSCGVKNRMHIRRKRIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNVGSSGGKVTYTSQEDLVEKKCLAKKYTHLSCDKVFCQPWQRCIEGTCVCKLPYQCPKNGTAVCATNRRSFPTYCQQKSLECLHPGTKFLNNGTCTAEGKFSVSLKHGNTDSEGIVEVKLVDQDKTMFICKSSWSMREANVACLDLGFQQGADTQRRFKLSDLSINSTECLHVHCRGLETSLAECTFTKRRTMGYQDFADVVCYTQKADSPMDDFFQCVNGKYISQMKACDGINDCGDQSDELCCKACQGKGFHCKSGVCIPSQYQCNGEVDCITGEDEVGCAGFASVTQEETEILTADMDAERRRIKSLLPKLSCGVKNRMHIRRKRIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 17-20 - skipped (4 entries)

SEQ ID NO: 21 - protein, 1156 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGSSGGKVTYTSQEDLVEKKCLAKKYTHLSCDKVFCQPWQRCIEGTCVCKLPYQCPKNGTAVCATNRRSFPTYCQQKSLECLHPGTKFLNNGTCTAEGKFSVSLKHGNTDSEGIVEVKLVDQDKTMFICKSSWSMREANVACLDLGFQQGADTQRRFKLSDLSINSTECLHVHCRGLETSLAECTFTKRRTMGYQDFADVVCYTQKADSPMDDFFQCVNGKYISQMKACDGINDCGDQSDELCCKACQGKGFHCKSGVCIPSQYQCNGEVDCITGEDEVGCAGFASVTQEETEILTADMDAERRRIKSLLPKLSCGVKNRMHIRRKRIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 22 - protein, 1376 aa, synthetic construct
METDTLLLWVLLLWVPGSTGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGSSGGKVTYTSQEDLVEKKCLAKKYTHLSCDKVFCQPWQRCIEGTCVCKLPYQCPKNGTAVCATNRRSFPTYCQQKSLECLHPGTKFLNNGTCTAEGKFSVSLKHGNTDSEGIVEVKLVDQDKTMFICKSSWSMREANVACLDLGFQQGADTQRRFKLSDLSINSTECLHVHCRGLETSLAECTFTKRRTMGYQDFADVVCYTQKADSPMDDFFQCVNGKYISQMKACDGINDCGDQSDELCCKACQGKGFHCKSGVCIPSQYQCNGEVDCITGEDEVGCAGFASVTQEETEILTADMDAERRRIKSLLPKLSCGVKNRMHIRRKRIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNVGGSSGGGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCII

SEQ ID NO: 23 - protein, 603 aa, Mus musculus
MKLAHLSLFLLALHLSSSRSPSASDLPQEELVDQKCLLQKYTHRSCNKVFCQPWQRCIEGTCICKLPYQCPRAGTPVCAMNGRSYPTYCHQKSFECLHPEIKFSHNGTCAAEGKFNVSLIYGRTKTEGLVQVKLVDQDERMFICKNSWSMAEANVACVDLGFPLGVRDIQGSFNISGNLHINDTECLHVHCRGVETSLAECAFTKRRTELSNGLAGVVCYKQDADFPTSLSFQCVNGKHIPQEKACNGVNDCGDQSDELCCKGCRGNASLCKSGVCIPDQYKCNGEVDCITGEDESRCEEDRQQNIPKGLARSAQGEAEIETEETEMLTPGMDNERKRIKSLLPKLSCGVKRNTHTRRKRVIGGKPANVGDYPWQVAIKDGQRITCGGIYIGGCWILTAAHCVRPSRAHSYQVWTALLDWLKPNSQLGIQTVKRVIVHEKYNGATFQNDIALIEMKMHTGKKECELPNSVPACVPWSPYLFQPNDRCIISGWGRGKDNQKVYSLRWGEVDLIGNCSQFYPDRYYEKEMQCAGTRDGSIDACKGDSGGPLVCEDINNVTYVWGIVSWGENCGKPEFPGVYTRVANYFDWISYHVGRSLVSQHNV

SEQ ID NO: 24 - protein, 20 aa, synthetic construct
  SITE 1: note = dimethylated
EDVPAADLSDQVPDTDSETR

SEQ ID NO: 25 - protein, 4 aa, synthetic construct
RRKR

SEQ ID NO: 26 - protein, 4 aa, synthetic construct
  REGION >4: note = The entire sequence of amino acids 1-4 can be repeated one or more times
GGSS

SEQ ID NO: 27 - protein, 6 aa, synthetic construct
  REGION >4: note = The entire sequence of amino acids 1-4 can be repeated one or more times
GGSSGG

SEQ ID NO: 28 - protein, 8 aa, synthetic construct
  REPEAT 1..4: note = single repeat of GGSS
  VARIANT 1..8: note = up to 1 copy of GGSS can be deleted
GGSSGGSS

SEQ ID NO: 29 - protein, 12 aa, synthetic construct
  REPEAT 1..6: note = single repeat of GGSSGG
  VARIANT 1..12: note = up to 1 copy of GGSSGG can be deleted
GGSSGGGGSSGG

SEQ ID NO: 30 - protein, 10 aa, synthetic construct
  REPEAT 1..4: note = single repeat of GGSS
  VARIANT 1..8: note = up to 1 copy of GGSS can be deleted
GGSSGGSSGG

SEQ ID NO: 31 - protein, 10 aa, synthetic construct
CAGFASVAQE

SEQ ID NO: 32 - protein, 26 aa, Mus musculus
CEEDRQQNIPKGLARSAQGEAEIETE

SEQ ID NO: 33 - protein, 18 aa, synthetic construct
GGSSGGSSGGSSGGSSGG

SEQ ID NO: 34 - protein, 10 aa, synthetic construct
GGSSGGSSGG

SEQ ID NO: 35 - protein, 6 aa, Homo sapiens
KVTYTS

SEQ ID NO: 36 - protein, 9 aa, Mus musculus
RSPSASDLP

SEQ ID NO: 37 - protein, 12 aa, synthetic construct
GGGGGGGGGGGG

SEQ ID NO: 38 - protein, 8 aa, synthetic construct
HHHHHHSG

SEQ ID NO: 39 - protein, 58 aa, synthetic construct
GGSSGGSSGGSSGGSSGGSSGGSSGGSSGGSSGGSSGGSSGGSSGGSSGGSSGGSSGG

SEQ ID NO: 40 - protein, 43 aa, synthetic construct
GGGGGGGGGGGGGGGGGGGGGGGGGGGGGGGGGGGGGGGGGGG

SEQ ID NO: 41 - protein, 42 aa, synthetic construct
GGGGSSGGGGSSGGGGSSGGGGSSGGGGSSGGGGSSGGGGSS

SEQ ID NO: 42 - protein, 44 aa, synthetic construct
GGSSGGSSGGSSGGSSGGSSGGSSGGSSGGSSGGSSGGSSGGSS

SEQ ID NO: 43 - protein, 53 aa, synthetic construct
GGGGGGGGGGGGGGGGGGGGGGGGGGGGGGGGGGGGGGGGGGGGGGGGGGGGG

SEQ ID NO: 44 - protein, 54 aa, synthetic construct
GGGGSSGGGGSSGGGGSSGGGGSSGGGGSSGGGGSSGGGGSSGGGGSSGGGGSS

SEQ ID NO: 45 - protein, 52 aa, synthetic construct
GGSSGGSSGGSSGGSSGGSSGGSSGGSSGGSSGGSSGGSSGGSSGGSSGGSS

SEQ ID NO: 46 - protein, 25 aa, synthetic construct
GGSSGGSSGGSSGGSSGGSSGGSSG

SEQ ID NO: 47 - protein, 15 aa, synthetic construct
GGSSGGSSGGSSGGG

SEQ ID NO: 48 - protein, 20 aa, synthetic construct
EDVPAADLSDQVPDTDSETR

SEQ ID NO: 49 - protein, 10 aa, synthetic construct
  SITE 1: note = dimethylated
  SITE 5: note = dimethylated
SEETKQNEGF

SEQ ID NO: 50 - protein, 10 aa, synthetic construct
SEETKQNEGF

SEQ ID NO: 51 - protein, 120 aa, synthetic construct
  REPEAT 1..6: note = single repeat of GGSSGG
  VARIANT 1..120: note = up to 19 copies of GGSSGG can be deleted
GGSSGGGGSSGGGGSSGGGGSSGGGGSSGGGGSSGGGGSSGGGGSSGGGGSSGGGGSSGGGGSSGGGGSSGGGGSSGGGGSSGGGGSSGGGGSSGGGGSSGGGGSSGGGGSSGGGGSSGG